(19) United States
(12) Patent Application Publication
(10) Pub. No.: US 2006/0015952 A1
(43) Pub. Date: Jan. 19, 2006

(54) Screening assays and methods of tumor treatment

(75) Inventor: Ellen H. Filvaroff, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/981,356

(22) Filed: Nov. 4, 2004

Related U.S. Application Data
(60) Provisional application No. 60/557,951, filed on Mar. 31, 2004.
(60) Provisional application No. 60/520,398, filed on Nov. 13, 2003.

(51) Int. Cl. A01K 67/027

(52) U.S. Cl. 800/10; 800/18

(57) ABSTRACT

The invention relates generally to the screening of candidate molecules for the treatment of tumor metastasis, and treatment methods using such molecules. Thus, the invention includes a method of screening comprising the steps of: (1) administering a plurality of test substances to a non-human syngeneic immunocompetent animal model bearing at least one soft tissue or bone metastasis, in the presence or absence of a primary tumor; (2) determining the effects of the test substances on the soft tissue or bone metastasis and growth of the primary tumor, if present; and (3) identifying a test substance that inhibits the growth of a soft tissue or bone metastasis, without adverse effect on the status of the primary tumor, if present.

### RELATED APPLICATIONS

This application is a non-provisional application filed under 37 CFR 1.53(b)(1), claiming priority under 35 USC 119(e) to provisional application No. 60/520,398 filed Nov. 13, 2003 and provisional application No. 60/557,951 filed Mar. 31, 2004, the contents of which are incorporated herein by reference.

### BACKGROUND OF THE INVENTION

1. Field of the Invention

This invention relates generally to the screening of candidate molecules for the treatment of tumor, including tumor metastasis, and treatment methods using such molecules.

2. Description of Related Art

Tumor and Cancer

The development of higher organisms is characterized by an exquisite pattern of temporal and spatially regulated cell division. Disruptions in the normal physiology of cell division are almost invariably detrimental. One such type of disruption is cancer, a disease that can arise from a series of genetic events.

Cancer cells are defined by two heritable properties, uncontrolled growth and uncontrolled invasion of normal tissue. A cancerous cell can divide in defiance of the normal growth constraints in a cell leading to a localized growth or tumor. In addition, some cancer cells also gain the ability to migrate away from their initial site and invade other healthy tissues in a patient. It is the combination of these two features that make a cancer cell especially dangerous. Cancer in humans develops through a multi-step process, indicating that multiple changes must occur to convert a normal cell into one with a malignant phenotype. One class of involved genes includes cellular oncogenes which, when activated by mutation or when expressed inappropriately, override normal cellular control mechanisms and promote unbridled cell proliferation.

An isolated abnormal cell population that grows uncontrollably will give rise to a tumor or neoplasm. As long as the neoplasm remains in a single location, it is said to be benign, and a complete cure may be expected by removing the mass surgically. A tumor or neoplasm is counted as a cancer if it is malignant, that is, if its cells have the ability to invade surrounding tissue. True malignancy begins when the cells cross the basal lamina and begin to invade the underlying connective tissue. Malignancy occurs when the cells gain the ability to detach from the main tumor mass, enter the bloodstream or lymphatic vessels, and form secondary tumors or metastases at other sites in the body. The more widely a tumor metastasizes, the harder it is to eradicate and treat.

As determined from epidermiological and clinical studies, most cancers develop in slow stages from mildly benign into malignant neoplasms. Malignant cancer usually begins as a benign localized cell population with abnormal growth characteristic called a dysplasia. The abnormal cells acquire abnormal growth characteristics resulting in a neoplasia characterized as a cell population of localized growth and swelling. If untreated, the neoplasia in situ may progress into a malignant neoplasia. Several years, or tens of years may elapse from the first sign of dysplasia to the onset of full-blown malignant cancer. This characteristic process is observed in a number of cancers.

Transforming Growth Factor-β(TGF-β)

TGF-β is a member of a large superfamily of growth factors (cytokines) involved in the regulation of various biological processes in organisms as diverse as drosophila and humans (Grande, Proc. Soc. Exp. Biol. Med., 214(1):27-40 (1997)). Such processes include cell proliferation and differentiation, extracellular matrix metabolism, bone morphogenesis, adhesion, apoptosis, cell migration, embryogenesis, tissue repair, and immune system modulation. Virtually every cell in the body (e.g., epithelial, endothelial, epithelial, hematopoietic, neuronal, and connective tissue cells) produces and has receptors for TGF-β.

There are multiple isoforms in the immediate TGF-β family, designated as TGF-β1, TGF-β2, TGF-β3, TGF-β4, and TGF-β5, with the mammalian isoforms being TGF-β1, TGF-β2, and TGF-β3. Each isoform is encoded by a distinct gene and is expressed in a tissue-specific and developmentally regulated manner. For example, TGF-β mRNA is broadly expressed in epithelial, endothelial, hematopoietic, and connective tissue cells, while TGF-β2 mRNA is primarily expressed in epithelial and neuronal cells, and TGF-β mRNA is primarily expressed in mesenchymal cells. The mammalian isoforms are highly conserved among species, indicating a critical biological function for each isoform. Despite their similarities, these isoforms differ in their binding affinities for TGF-β receptors.

The phenotypes resulting from the knockout of three mammalian TGF-β isoforms TGF-β1, TGF-β2 and TGF-β3 are very distinct and not overlapping. TGF-β1 null mice have an autoimmune-like inflammatory disease, TGF-β2 knockout mice exhibit perinatal mortality and severe development defects and TGF-β3-deficient mice have cleft palate and are defective in lung development. This indicates that these ligands have isoform-specific activities that cannot be compensated by other family members.

Members of the TGF-β family initiate their cellular action by binding to three high-affinity receptors designated as types I, II, and III (endoglin is another TGF-β receptor that is abundant on endothelial cells). The type III receptors (also called beta glycan), the most abundant type when present, function by binding all three TGF-β isoforms and then present them to the signaling receptors, type I and II. The soluble extracellular domain of the type III receptor can function as a TGF-β antagonist. (Vilchis Landeros et al., Biochem. J., 355:215-222 (2001)). The intracellular domains of the type I and II receptors contain serine/threonine protein kinases, which initiate intracellular signaling by phosphorylating several signal-transduction proteins referred to as “SMADS” (this term was derived from the Sma and MAD gene homologues identified in Caenorhabditis elegans and Drosophila melanogaster). Although TGF-βs may bind the type III receptor, which then presents the TGF-β to the type I and II receptors, TGF-β1 and TGF-β3 are also capable of directly binding the type II receptors. Following binding of ligand to the type II receptors, the type II receptor recruits, binds, and transphosphorylates the type I receptors, thereby stimulating the protein kinase activity of the receptors. In this general manner, TGF-βs initiate signal transduction.

TGF-β1 is quantitatively the major isoform, but essentially every tissue expresses one or more of the three isoforms, together with their cognate receptors. Expression patterns of the three isoforms differ spatially and temporally, both during development and in the adult animal, indicating that they play non-redundant roles. In support of this concept, knockout mice for the three isoforms have non-overlapping spectra of phenotypes. All three TGF-βs are clearly important in development, since knocking out any of these genes causes some embryonic or perinatal lethality. Additional roles in the adult animal can be inferred from the expression patterns of the TGF-βs (both in the unperturbed animal and in response to challenge), from the phenotypes of mice in which TGF-β function has been compromised (either through genetic manipulation or the application of TGF-β antagonists), and from in vitro studies showing effects of TGF-β on different specialized cell types. Thus, TGF-βs play key roles in regulating cell proliferation, differentiation and programmed cell death, immune system function, angiogenesis, and tissue repair. Consequently, many disease processes are associated with aberrant TGF-β function. Loss of TGF-β function has been implicated in the pathogenesis of cancer, atherosclerosis and autoimmune disease, while excessive TGF-β production has been implicated in fibroproliferative disorders, in parasite-induced immunosuppression, and in metastasis (for review, see e.g., Roberts and Sporn, The Transforming Growth Factors-β, in Sporn and Roberts (eds.), Handbook of Experimental Pharmacology: Peptide Growth Factors and Their Receptors, Springer Verlag, Berlin (1990), at pages 419-472; Flanders and Roberts, Transforming Growth Factor-β, in Oppenheim and Feldmann, Cytokine Reference, Academic Press, London (2000); Dunker and Krieglstein, Eur. J. Biochem., 267:6982-6988 (2000); Branton and Kopp, Microbes Infect., 1:1349-1365 (1999); and Chen and Wahl, Microbes Infect., 1:1367-1380 (1999)).

Increases and decreases in TGF-β have been associated with numerous diseases, including atherosclerosis and fibrotic diseases of the kidney, liver, and lung. Genetic mutations in TGF-β, its receptors, and/or intracellular signaling molecules associated with TGF-β are also important in pathogenic processes, particularly in cancer and hereditary hemorrhagic telangiectasia.

The TGF-β isoforms play a complex role during the tumorigenesis of various tumors. In many cases, the tumor cells become resistant to TGF-β, which is often due to mutations within genes encoding (a) the receptor, (b) molecules directly involved in signaling (SMADS) or (c) downstream proteins, which play a crucial role in the control of cell cycle (e.g. CDK-inhibitors, Rb protein etc.). Moreover, several studies report on enhanced secretion of TGF-β in tumor cells leading to the inhibition of proliferation of adjacent tissue. This enhanced secretion of TGF-β might also promote angiogenesis (stimulation of the production of VEGF). Both effects stimulate tumor growth.

TGF-β is a pleiotropic cytokine that can affect tumor growth both directly (by affecting cell growth and differentiation) and indirectly (by modulating the immune system, extracellular matrix turnover, and angiogenesis). Previous data have shown that tumor cells can change their response to TGF-β from its being growth inhibitory in early-stage tumors to being pro-metastatic in later-stage tumors. The TGF-β signaling pathway has been considered both as a tumor suppressor pathway and a promoter of tumor progression and invasion. For a review see, e.g., Derynck et al., Nat. Genet., 29(2):117-129 (2001).

In normal cells, TGF-β can act as a tumor suppressor by inhibiting cellular proliferation and/or by promoting cellular differentiation or apoptosis. During the course of tumorigenesis, many cells lose their TGF-β-mediated growth inhibition. After development of resistance to growth inhibition by TGF-β, tumor cells and stromal cells within tumors often increase their production of TGF-β. This increased TGF-β production is associated with increased invasiveness and metastasis of tumor cells to distant organs, at least partially due to TGF-β-mediated stimulation of angiogenesis, cell motility, immunosuppression, and an altered interaction of tumor cells with the extracellular matrix. Thus, tumor cell resistance to TGF-β and concomitant overproduction of the TGF-β ligand results in enhancement of tumor formation and greater aggressiveness of those tumor cells. Indeed, TGF-β and the associated receptors play a very important role in health and disease.

TGF-βs are potent inhibitors of epithelial cell proliferation, and the TGF-β system has tumor suppressor activity in many tissues (for review, see e.g., Gold, Crit. Rev. Oncol., 10:303-360 (1999); Massague et al., Cell, 103:295-309 (2000); and Akhurst and Balmain, J. Pathol., 187:82-90 (1999)). Reduction or loss of TGF-β receptors or downstream signaling components is observed in many human tumor types, including tumors of the gastrointestinal tract, breast and prostate. Studies using genetically engineered mouse models or xenografts of genetically manipulated tumor cell lines have confirmed a causal connection between diminished TGF-β function and increased tumorigenesis. However, the role of TGF-βs in tumorigenesis is complex, as many late-stage human tumors show increased expression of TGF-β, which is associated with increased metastasis and poor prognosis. It appears that TGF-βs function as tumor suppressors early in tumorigenesis, but that in the later stages, they may function as oncogenes and promote the development of aggressive metastatic disease. The mechanism for promotion of metastasis is thought to include enhanced tumor cell invasiveness, enhanced angiogenesis and suppression of the immune surveillance system. TGF-β1 is the isoform that is most commonly upregulated in late-stage human cancer, though TGF-β2 and TGF-β3 have been implicated in some instances.

TGF-β expression is increased in many advanced human cancers and is correlated with enhanced invasion and/or metastasis. TGF-β1 and TGF-β3 are the isoforms that are usually involved. Frequently, plasma levels of the TGF-βs are also increased in cancer patients with advanced disease, indicating that the tumors are secreting significant amounts of TGF-β into the circulation. Tumors showing elevated TGF-β expression include breast, colon, gastric, liver, pancreatic, prostate, lung, kidney, bladder and nasopharyngeal carcinomas, melanomas, chondrosarcomas and osteosarcomas.

Immunohistochemical staining for TGF-β1 associates with disease progression in human breast cancer (Gorsch et al., Canc. Res., 52:6949-6952 (1992)), and correlates with node positivity and metastasis (Walker and Dearing, Eur. J. Canc., 28:641-644 (1992)). Secreted extracellular TGF-β1 protein is increased at the advancing edge of primary human breast carcinomas and in lymph node metastases (Dalal et al., Am. J. Pathol., 143:381-389 (1993)). TGF-β1 is increased in the plasma of 81% newly-diagnosed breast cancer patients, and levels are normalized by surgical resection in node-negative patients, but not in node-positive patients, suggesting that primary tumors and metastases secrete significant quantities of TGF-β1 into the circulation (Kong et al., Ann. Surg., 222:155-162 (1995)). Increased plasma levels of TGF-β3 have also been found in breast cancer patients with positive lymph nodes (Li et al., Intl. J. Canc., 79:455-459 (1998)), and the combination of lymph node involvement and positive TGF-β3 expression in the invasive tumor has been associated with poor prognosis (Ghellal et al., Anticancer Res., 20:4413-4418 (2000)).

For colon cancer patients, intense staining for TGF-β1 in the resected primary tumor has been significantly correlated with disease progression to metastasis (Friedman et al., Canc. Epidemiol. Biomarkers Prev., 4:549-554 (1995)). In addition, increased levels of TGF-β1 staining have been found in the cancer cells invading local lymph nodes when compared with the primary tumor, and elevated TGF-β1 was implicated in the metastatic process in 75% of the cases examined (Picon et al., Canc. Epidemiol. Biomarkers Prev., 7:497-504 (1998)). Plasma TGF-β1 and TGF-β2 levels are increased in patients with colorectal cancer and levels are higher in more advanced disease (Tsushima et al., Gastroenterol., 110:375-382 (1996); and Bellone et al., Eur. J. Canc., 37:224-233 (2001)). Similarly, elevated plasma TGF-β1 levels were seen in patients with hepatocellular carcinoma, and levels were normalized following resection of the tumor, indicating that the tumor was the source of the TGF-β1 (Shirai et al., Jpn. Canc. Res., 83:676-679 (1992)). Positive staining for TGF-β1 in gastric cancer tissues is closely related to serosal invasion and lymph node metastasis (Maehara et al., J. Clin. Oncol., 17:607-614 (1999)), and elevated serum levels of TGF-β1 correlate with lymph node metastasis and poor prognosis (Saito et al., Anticancer Res., 20:4489-4493 (2000)). In addition, mRNAs for TGF-β1, 2 and 3 are increased in 50% of pancreatic cancer cases and the increased expression correlates with decreased survival (Friess et al., Gastroenterol., 105:1846-1856 (1993)).

Increased TGF-β1 staining is associated with higher tumor grade and metastasis in prostate cancer patients (Wikstrom et al., Prostate, 37:19-29 (1998)). Increased TGF-β1 staining is a negative predictive factor for patient survival (Stravodimos et al., Anticancer Res., 20:3823-3828 (2000)). Primary tumors that had metastasized have shown higher levels of staining for TGF-β1 than those that had not metastasized (Eastham et al., Lab. Invest., 73:628-635 (1995)). Furthermore, plasma TGF-β1 levels are significantly elevated in patients with clinically evident metastases (Adler et al., J. Urol., 161:182-187 (1999)), or with primary stage III/IV disease (Ivanovic et al., Nat. Med., 1:282-284 (1995)).

Increased extractable TGF-β1 protein was found in the primary tumors of lung cancer patients with lymph node metastasis compared with those without metastasis (Hasegawa et al., Canc., 91:964-971 (2001)). Elevated plasma levels of TGF-β1, and to a lesser extent TGF-β2, are found in melanoma patients with disseminated but not loco-regional disease (Krasagakis et al., Br. J. Canc., 77:1492-1494 (1998)). In osteosarcomas, elevated immunohistochemical staining for TGF-β1 or TGF-β3 is associated with a higher rate of subsequent lung metastasis (Yang et al., J. Exp. Med., 184:133-142 (1998)). Plasma TGF-β1 levels are also significantly elevated in patients with chondrosarcomas (Gridley et al., Canc. Detect. Prev., 22:20-29 (1998)), and renal cell carcinomas (Wunderlich et al., Urol. Intl., 60:205-207 (1998); and Junker et al., Cytokine, 8:794-798 (1996)), suggesting that these types of tumors secrete high levels of TGF-β. Serum TGF-β1 levels are increased in patients with invasive but not superficial bladder cancer, although no further increase is found in patients with metastatic disease (Eder et al., J. Urol., 156:953-957 (1996)). Serum TGF-β1 is also increased in patients with Epstein-Barr virus-associated nasopharyngeal carcinoma, particularly in patients with relapsing tumors (Xu et al., Intl. J. Canc., 84:396-399 (1999)).

Pretreatment in serum-free culture of a rat mammary adenocarcinoma cell line with TGF-β1 protein was found to cause a significant increase in the number of lung metastases following injection into syngeneic rats (Welch et al., Proc. Natl. Acad. Sci. USA, 87:7678-7682 (1990)). Transfection of primary human prostate tumor cells with the TGF-β1 gene was found to stimulate metastasis after orthotopic implantation in SCID mice (Stearns et al., Canc. Res., 5:711-720 (1999)). Similar results were obtained with rat prostate cancer cells (Steiner and Barrack, Mol. Endocrinol., 6:15-25 (1992)) and Chinese hamster ovary cells (Ueki et al., Jpn. J. Canc. Res., 84:589-593 (1993)).

Treatment of athymic mice with neutralizing antibodies to TGF-β1, 2, and 3 has been found to suppress the formation of lung metastases following intraperitoneal inoculation with the human breast cancer cell line MDA-MB-231 (Arteaga et al., J. Clin. Invest., 92:2569-2576 (1993)). The same antibody caused a three-fold decrease in the number of metastases formed when B16F1 melanoma cells were injected into the tail vein of syngeneic mice (Wojtowicz-Praga et al., J. Immunother. Emphasis Tumor Immunol., 19:169-175 (1996)). In other reports, an anti-TGF-β1 monoclonal antibody was found to decrease the development of metastases following subcutaneous implantation of human carcinoma cell lines into athymic mice (Hoefer and Anderer, Canc. Immunol. Immunother., 41 :302-308 (1995)). In all three of these studies, suppressive effects of TGF-β on immunosurveillance by natural killer cells, monocytes or lymphokine-activated killer cells of the host animal were implicated in the increased metastatic efficiency. In addition, treatment of malignant mouse fibrosarcoma cells with specific antisense oligonucleotides to TGF-β1 significantly decreased the metastatic properties of these cells, suggesting that TGF-β produced by the tumor cell itself is important in promoting metastasis (Spearman et al., Gene, 149:25-29 (1994)).

In three different experimental systems, interfering with the responsiveness of a mammary tumor cell line to TGF-β by transfection with a dominant negative type II TGF-β receptor has caused a significant decrease in the metastatic efficiency of these cells (McEarchem et al., Int. J. Canc., 91:76-82 (2001); Oft et al., Curr. Biol., 8:1243-1252 (1998); and Yin et al., J. Clin. Invest., 103:197-206 (1999)). In the case of the human breast cancer cell line MDA-MB-23 1, bony metastases were significantly reduced and survival was prolonged in a xenograft model using athymic mice (Yin et al., supra). These results suggest that, at least in breast cancer, TGF-β acting directly on the tumor cell can increase metastatic efficiency. Mechanisms include enhanced invasiveness and increased production of parathyroid hormone-related peptide.

TGF-β is not uniformly pro-metastatic however, as pretreatment with TGF-β has been reported to inhibit formation of pulmonary metastases by Chinese hamster chondrosarcoma cells (Fujisawa et al., J. Exp. Med., 187:203-213 (2000)), transfection with TGF-β3 reduced metastatic dissemination of rat oral carcinoma cell lines (Davies et al, J. Oral. Pathol. Med., 29:232-240 (2000)), and overexpression of the type II TGF-β receptor reduced the metastatic potential of K-ras-transformed thyroid cells (Turco et al., Intl. J. Canc., 80:85-91 (1999)). This suggests that the ability of TGF-β to promote metastasis may vary with tumor type.

Since TGF-βs play such important roles in maintaining normal cellular homeostasis in many organ systems, a key conceptual problem with the use of TGF-β antagonists to treat TGF-β-driven pathologies has been the likelihood of undesired side-effects on the normal tissues, including but not limited to aberrant cell proliferation and increased tumor formation due to loss of tumor suppressor function of TGF-βs in many epithelia, as well as problems due to dysregulation of the immune system (e.g., multifocal inflammation, autoimmune manifestations and myeloid hyperplasia). These pathologies are predicted based on studies of mice with experimentally compromised TGF-β function.

TGF-β1 null mice on a Rag2 null genetic background that permits extended survival develop non-metastatic colon cancer (Engle et al., Canc. Res., 59:3379-3386 (1999)), consistent with the idea that endogenous TGF-β1 functions as a tumor suppressor in the colonic epithelium. TGF-β1+/− mice with only one functional TGF-β1 allele show hyperplasia of the glandular stomach (Boivin et al., Lab. Invest., 74:513-518 (1996)), and an increased susceptibility to carcinogen-induced tumorigenesis in the liver and lung (Tang et al., Nat. Med., 4:802-807 (1998)). Similarly, interfering with TGF-β responsiveness by targeted overexpression of a dominant negative TGF-β receptor causes hyperplasia and increased susceptibility to carcinogen-induced tumorigenesis in the skin and mammary gland (Amendt et al., Oncogene, 17:25-34 (1998); and Bottinger et al., Canc. Res., 57:5564-5570 (1997)), and an increase in spontaneous mammary tumorigenesis (Gorska et al., Proc. Am. Assoc. Canc. Res., 42:422 (2001)).

Soon after weaning, TGF-β null mice die of a rapid wasting syndrome associated with a multifocal inflammatory response leading to massive infiltration of lymphocytes and macrophages into many organs, particularly the heart and lungs (Shull et al., Nature, 359:693-699 (1992); and Kulkarni et al., Proc. Natl. Acad. Sci. USA, 90:770-774 (1993)). The syndrome has many of the hallmarks of autoimmune disease, including circulating antibodies to nuclear antigens, immune complex deposition and enhanced expression of major histocompatibility complex antigens (MHCI and MHCII) (Dang et al., J. Immunol., 155:3205-3212 (1995)). In MCH-deficient backgrounds in which the inflammation is suppressed, there is a myeloid hyperplasia (Letterio et al., J. Clin. Invest., 98:2109-2119 (1996)). These studies suggest key roles for TGF-β1 in maintaining normal homeostasis in multiple compartments of the immune system. Consistent with this, reduction in TGF-β responsiveness by transgenic expression of a dominant negative TGF-β receptor in CD4+ and CD8+ T-cells causes T-cell differentiation into effector T-cells, which also leads to an autoimmune-like syndrome (Gorelik and Flavell, Immun., 12:171-181 (2000)), while expression of the dominant negative receptor in early T-cells gave rise to a CD8+ T cell lymphoproliferative disorder resulting in the massive expansion of the lymphoid organs (Lucas et al., J. Exp. Med., 191:1187-1196 (2000)).

TGF-β antagonists (antibodies, SR2F discussed below, antisense TGF-β DNA and dominant negative TGF-β receptors) have been previously used to treat TGF-β-driven pathologies, especially fibrosis, in a number of animal model systems. However, these have generally been relatively short-term experiments, frequently involving local delivery of the antagonist, and the consequences of long-term exposure to TGF-β antagonists have not been assessed, particularly regarding tumorigenesis and immune system function.

Overexpression of TGF-βs has been implicated in the pathogenesis of a number of diseases, particularly fibrotic disorders and late-stage cancer. Initial studies using TGF-β antagonists used anti-TGF-β antibodies or naturally occurring TGF-β binding proteins. For example, both anti-TGF-β antibodies and the proteoglycan decorin, which is a TGF-β binding protein, have been used successfully in a rat model to protect against experimental kidney fibrosis (Border et al., Nature, 360:361-364 (1992); and Border et al., Nature, 346:371-374 (1990)).

TGF-βs are synthesized as biologically latent complexes that must be activated before they can bind to the signaling receptor complex. Latency is conferred by non-covalent association of the cleaved precursor pro-region of the TGF-β pro-peptide with the mature TGF-β. The precursor pro-region is also known as the latency-associated peptide (LAP), and purified TGF-β1 LAP can function as an antagonist for all three TGF-β isoforms (Bottinger et al., Proc. Natl. Acad. Sci. USA, 93:5877 (1996)).

In general, antibody and binding protein-based antagonists have been relatively low affinity. The extracellular ligand-binding domain of the type II TGF-β receptor has high affinity binding sites for TGF-β1 and TGF-β3 (O'Connor-McCourt et al., Ann. N.Y. Acad. Sci., 766:300-302 (1995)). The affinity is further increased when the soluble extracellular ligand-binding domain is fused to the Fc domain of human immunoglobulin, which causes dimerization of the ligand-binding domain. Addition of an Fc domain to soluble cytokine receptors also increases their in vivo half-life (Capon et al., Nature, 337:525-531 (1989)). A soluble TGF-β receptor-Fc fusion protein (SR2F) has been generated in a number of labs, and has been used successfully to block or reduce liver fibrogenesis induced by dimethylnitrosamine or by ligation of the common bile duct in rats, fibrosis in an experimental glomerulonephritis model, radiation-induced enteropathy in mice, bleomycin-induced lung fibrosis in hamsters, and adventitial fibrosis and intimal lesion formation in a rat balloon catheter denudation model (Ueno et al., Hum. Gene Ther., 11:33-42 (2000); George et al., Proc. Natl. Acad. Sci. USA, 96:12719-12724 (1999); Isaka et al., Kidney Intl., 55:465-475 (1999); Zheng et al., Gastroenterol., 110:1286-1296 (2000); Wang et al., Thorax, 54:805-812 (1999); and Smith et al., Circ. Res., 84:1212-1222 (1999)). In most cases, the SR2F antagonist was given as injections of purified protein, though in two cases it was given in a gene therapy approach by introduction of the cDNA into the muscle (Ueno et al., supra; and Isaka et al., supra). None of the authors noted untoward side effects, but all were relatively short-term studies.

TGF-β is synthesized in a biologically latent form that must be activated before the TGF-β can bind to the receptor and elicit a biological response (Munger et al., Kidney Intl., 51:1376-1382 (1997)). Relatively little is known about the mechanism and circumstances of TGF-β activation in vivo, due to difficulties in discriminating between and experimentally quantitating active and latent TGF-β. Using an immunofluorescence technique that distinguishes active and latent TGF-β in frozen tissue sections, it has recently been shown for the mammary gland, that activation of latent TGF-β may occur very locally on a cell-by-cell basis in epithelium of the normal tissue (Barcellos-Hoff and Ewan, Breast Canc. Res., 2:92-99 (2000)). In contrast, in the face of pathologic challenge, there may be much more widespread activation of latent TGF-β. For example, irradiation of the mammary gland caused extensive activation of TGF-β both in the epithelium, the peri-epithelial stroma and the adipose stroma (Barcellos-Hoff et al., J. Clin. Invest., 93:892-899 (1994)). Similarly, the majority of normal cells in culture secrete predominantly latent TGF-β, though cells from more advanced tumors secrete higher amounts of active TGF-β. Significantly, in studies with oncogene-transformed fibrosarcoma cell lines, the highly metastatic fibrosarcomas were distinguished by secreting a much higher fraction of the TGF-β in the active form, although all transformed lines secreted high levels of total TGF-β (Schwarz et al., Growth Factors, 3:115-127 (1990)).

U.S. patent application publication No. 2002/0176758, published on Nov. 28, 2002, and U.S. Pat. Nos. 5,571,714; 5,772,998; 5,783,185; and 6,090,383 disclose monoclonal antibodies to TGF-β and various uses of such antibodies.

U.S. patent application publication No. 2003/0125251, published Jul. 3, 2003, discloses that a TGF-β antagonist selectively neutralizes “pathological” TGF-β. Specifically, it provides methods and compositions for the suppression of metastasis by a soluble TGF-β antagonist (SR2F). This antagonist is composed of the soluble extracellular domain of the type II TGF-β receptor fused to the Fc domain of human IgG. In particular, this application is directed to the use of SR2F to prevent metastasis without affecting the normal physiological role of TGF-β. Thus, the SR2F discriminates between “physiological” TGF-β and “pathological” TGF-β in such a manner that only the “pathological” TGF-β is affected by the administration of SR2F. It also discloses a transgenic non-human animal comprising a soluble TGF-beta antagonist, and preferably wherein said soluble TGF-beta antagonist prevents metastasis of tumors in said transgenic animal.

U.S. patent application publication No. 2003/0028905, published Feb. 6, 2003, relates to gene expression in normal cells and cells of tumors and particularly to mutant forms of the TGF-β II receptor that bind all TGF-β isoforms. It further relates to diagnostic and therapeutic methods useful for diagnosing and treating a disease associated with mutated TGF-β type II receptor, e.g. a tumor, and to a transgenic non-human animal characterized in that it contains an insertion of TGF-β1 encoding cDNA within the first exon of the TGF-β2 encoding gene.

While the absence of elastic fibers in the lung and colon underscores the structural requirement of latent TGF-beta binding protein (LTBP4), the lack of extracellular TGF-beta implicates LTBP4 in TGF-beta signaling. As TGF-beta inhibits epithelial cell proliferation, particularly in the colon, it can be concluded that its absence from the colonic ECM is the most likely oncogenic trigger for the development of colon cancer in mice. Indeed, several studies have associated defects in TGF-beta signaling with colorectal cancer. For example, mice with null mutations in the TGF-beta-signal-transducing protein, Smad 3, develop tumors that are similar to the tumors as growing in 3C7 mice (Zhu et al., Cell, 94: 703-714 (1998)). Furthermore, mutations in the TGF-signal-transducing proteins Smad 2 and Smad 4 or mutations in the TGF-beta3 type II receptors are very common in human colorectal cancers, suggesting that TGF-beta3 and its downstream targets have tumor suppressor functions (Markowitz et al., Science, 268: 1336-1338 (1995); Riggins et al., Cancer Res., 57: 2578-2580 (1997); Zhou et al., Proc. Natl. Acad. Sci. USA, 95: 2412-2416 (1998)).

WO 2003/015505 discloses an animal model demonstrating a dual function of the TGF-beta binding proteins. Such animal model does not produce functional latent LTBP or produces suboptimal levels of latent transforming growth factor binding protein LTBP. This reference also discloses methods and kits for diagnosing cancer, pulmonary emphysema or cardiomyopathy and analyzing whether cancer and/or pulmonary emphysema and/or cardiomyopathy are caused by a differential expression of LTBP or by a defect in the LTBP-4 gene. This patent application reports that LTBP-4 is important for the integrity of the ECM and prevents oncogenic transformation, cancer cell invasion and metastatic spread

U.S. Pat. Nos. 6,455,757 and 6,175,057 feature non-human transgenic animal models for Alzheimer's disease (AD) and CM, wherein the transgenic animal is characterized by 1) expression of bioactive transforming growth factor-β1 (TGF-β1) or 2) both expression of bioactive TGF-β1 and expression of a human amyloid β precursor protein (APP) gene product.

With advances in detection and treatment of primary tumors, mortality in cancer patients is increasingly linked to the existence of secondary tumors (metastases). Cancer is believed to be incurable once the patient has bone metastases.

Many steps are involved in metastasis of tumor cells from the primary site to secondary sites. Animal studies are essential for understanding the effects of various compounds on primary and secondary tumors. Unfortunately, many tumor cells do not metastasize in animal models, especially not to bone.

Therefore, a need exists for methods of screening using animal model systems allowing one to distinguish between the growth inhibitory and pro-metastatic activities of TGF-β.

There is further a need for developing screening assays to identify molecules suitable for the treatment of secondary tumors.

In addition, a need exists for developing new approaches for the treatment of cancer, in particular advanced metastatic cancer, which recognize and address the different responsiveness of different types and stages of primary and metastatic tumors to TGF-β and TGF-β inhibitors or antagonists. There is a particular need for identifying a population of patients diagnosed with advanced, metastatic cancer that is likely to respond well to treatment with TGF-β inhibitors or antagonists.

There is a further need to develop treatments for bone metastasis, and bone destruction and/or bone loss, whether or not associated with a primary tumor.

### SUMMARY OF THE INVENTION

Accordingly, the invention is as claimed. In one aspect, the present invention concerns a method of screening comprising the steps of: (1) administering a plurality of test substances to a non-human syngeneic immunocompetent animal model bearing at least one soft tissue or bone metastasis, in the presence or absence of a primary tumor; (2) determining the effects of said test substances on the soft tissue or bone metastasis and growth of the primary tumor, if present; and (3) identifying a test substance that inhibits the growth of a soft tissue or bone metastasis, without adverse effect on the status of the primary tumor, if present.

In another aspect, the invention concerns a method of determining if a mammalian patient diagnosed with cancer is likely to benefit from treatment with a TGF-beta antagonist, comprising:
(a) testing the sensitivity of cancer cells obtained from the patient to the growth-inhibitory effect of TGF-beta;
(b) obtaining a gene expression profile of the cancer cells obtained from the patient and comparing it with a gene expression profile of cancer cells obtained from an animal model that are responsive to treatment with a TGF-beta antagonist; and
(c) identifying the patient as likely to benefit from treatment with a TGF-beta antagonist if the cancer cells obtained from the patient are not sensitive to the growth-inhibitory effect of TGF-beta and have a gene expression profile similar to the gene expression profile of the cancer cells obtained from said animal model that are responsive to said treatment.

If the cancer is breast cancer, including primary and metastatic breast cancers, the foregoing prognostic method may additionally include the step of determining the Her2 status of the patient, where Her2⁺ patients typically, although not always, are likely not to respond, or to respond poorly, to treatment with a TGF-beta antagonist alone.

Methods of treating cancer in patients identified as likely to benefit from treatment with a TGF-beta antagonist with such antagonists are also within the scope of the invention.

In a further aspect, the invention concerns a method of treating bone destruction or bone loss associated with a tumor metastasis in a mammalian patient comprising administering to the patient an effective amount of a TGF-beta antagonist.

In yet another aspect, the invention concerns a method for treating a mammalian patient diagnosed with cancer comprising administering to the patient an effective amount of a combination of a TGF-beta antagonist and a chemotherapeutic or cytotoxic agent, and monitoring the response of the patient to the combination, wherein the effective amount of said combination is lower than the sum of the effective amounts of said TGF-beta antagonist and said chemotherapeutic or cytotoxic agent when administered individually, as single agents. If the cancer is breast cancer, including metastatic breast cancer, the chemotherapeutic agent may, for example, be a taxoid such as paclitaxel (Taxol®) or a taxol derivative (e.g., doxetaxel (Taxotere®)).

In place of or in addition to the chemotherapeutic or cytotoxic agent, the patient diagnosed with metastatic cancer may be administered a TGF-beta antagonist and be exposed to radiation therapy. Specifically, the invention also concerns a method for treating a mammalian patient diagnosed with cancer comprising administering to the patient an effective amount of a combination of a TGF-beta antagonist and radiation therapy, wherein the effective amount of said combination is lower than the sum of the effective amounts of said TGF-beta antagonist and said radiation therapy when administered individually, as single agents. The cancer is preferably breast or metastatic breast cancer or colorectal cancer, and the method may additionally comprise administering an anti-angiogenic agent to the patient.

In a still further aspect, the invention relates to a method for treating a mammalian patient diagnosed with cancer comprising administering to the patient an effective amount of a combination of a TGF-beta antagonist and an anti-angiogenic agent, and monitoring the response of the patient to the combination. In one preferred embodiment, the anti-angiogenic agent is an antibody specifically binding vascular endothelial growth factor, and/or the TGF-beta antagonist is an antibody specifically binding TGF-beta. In another preferred embodiment, the method additionally comprises administering to the patient an effective amount of a chemotherapeutic or cytotoxic agent. In another aspect, this method is one wherein the effective amount of said combination is lower than the sum of the effective amounts of said TGF-beta antagonist and said anti-angiogenic agent when administered individually, as single agents.

In a still further aspect, the invention provides a method for treating a mammalian patient diagnosed with cancer and predetermined not to respond, or to respond poorly, to a TGF-β antagonist, comprising administering to the patient an effective amount of a combination of a TGF-β antagonist and a chemotherapeutic or cytotoxic agent, or a combination of a TGF-β antagonist and radiation therapy, and monitoring the response of the patient to the combination. In one preferred embodiment, the cancer is breast cancer. In another preferred embodiment, the chemotherapeutic agent is a taxoid.

In yet another aspect, the invention relates to a kit comprising a container comprising an antibody specifically binding vascular endothelial growth factor, a container comprising an antibody specifically binding TGF-beta, and instructions for use of both antibodies in combination in effective amounts to treat cancer in a mammalian patient.

### BRIEF DESCRIPTION OF DRAWINGS

FIGS. 1A and 1B show the effect of an anti-TGF-β antibody on primary tumor growth (FIG. 1A) and plasma VEGF (FIG. 1B) levels in a 4T1 mouse mammary carcinoma model.

FIG. 2 shows the histology scores of secondary lung tumors in a 4T1 mouse mammary carcinoma model, following treatment with an anti-TGF-β antibody, relative to control.

FIG. 3 shows the tissue weights of secondary lung tumors in a 4T1 mouse mammary carcinoma model, following treatment with an anti-TGF-β antibody, relative to control.

FIG. 4 shows the computed tomography (CT) values of secondary lung tumors in a 4T1 mouse mammary carcinoma model, following treatment with an anti-TGF-β antibody (darker bar), relative to control (lighter bar).

FIGS. 5A and 5B show MicroCT (x-ray microtomography) images of normal trabecular bone (FIG. 5A) and bone metastasis (FIG. 5B) resulting from the spread of primary tumor in a 4T1 mouse mammary carcinoma model.

FIG. 6 depicts the effect of anti-TGF-β antibody treatment on primary tumor growth in a mouse model of trastuzumab (HERCEPTIN®)-sensitive Her2+breast cancer (cell line F2-1282).

FIG. 7 depicts the effect of anti-TGF-β antibody treatment on plasma VEGF levels in a mouse model of trastuzumab-sensitive Her2⁺ breast cancer (cell line F2-1282).

FIG. 8 depicts the effect of anti-TGF-β antibody treatment on primary tumor growth in a mouse model of trastuzumab-resistant Her2⁺ breast cancer (cell line Fo5).

FIG. 9 depicts the effect of anti-TGF-β antibody treatment on plasma VEGF levels in a mouse model of trastuzumab-resistant Her2⁺ breast cancer (cell line Fo5).

FIGS. 10 and 11 illustrate that treatment with an anti-TGF-β antibody increases survival in two mouse models of melanoma (F10 and BL6, respectively).

FIGS. 12 and 13 are images of secondary lung tumors in a mouse model of melanoma (MicroCT and light image, respectively).

FIGS. 14 and 15 are images of secondary lung tumors in a mouse model of melanoma (MicroCT and light images, respectively).

FIG. 16 shows that treatment with an anti-TGF-β antibody decreases the number of secondary lung tumors in a mouse model of melanoma.

FIG. 17 shows that treatment with an anti-TGF-β antibody decreases the incidence of lung tumors in a mouse model of melanoma.

FIGS. 18A and 18B show the effect of treatment with an anti-TGF-β antibody on the volume (FIG. 18A) and weight (FIG. 18B) of PyMT tumors, relative to an IgG control. In FIG. 18B, the right-hand bar is anti-TGF-β antibody and the left-hand bar is the IgG control.

FIGS. 19A and 19B depict alignments of the amino acid sequences of the variable light (V_{L}) (FIG. 19A) and variable heavy (V_{H}) (FIG. 19B) domains of murine monoclonal antibody 2G7 (SEQ ID Nos. 1 and 2, respectively); V_{L} and V_{H} domains of humanized huxTGFB version (V5H.V5L) (SEQ ID Nos. 3 and 4, respectively), and human V_{L} and V_{H} consensus frameworks (hum κ1, light kappa subgroup I; humIII, heavy subgroup II) (SEQ ID Nos. 5 and 6, respectively). Asterisks identify differences between humanized huxTGFB and murine monoclonal antibody 2G7 or between humanized huxTGFB and the human consensus framework regions. Complementarity Determining Regions (CDRs) are underlined, and the CDRs of the actual human germ line sequence are below the consensus framework regions for comparison (SEQ ID NOS: 7-11).

FIG. 20 shows the DNA sequences (SEQ ID NOS: 12-17) encoding the various CDR regions (SEQ ID NOS: 18-23).

FIG. 21 shows the amino acid sequences of 709.1 and H.IgG1 (SEQ ID NO:24); of H₂NI.V5L (SEQ ID NO:25), of V11H.V11 L (SEQ ID NO:26), of V5H.V5L (SEQ ID NO:27), of chimL.chimH (SEQ ID NO:28), and of V5H.g1L2 (SEQ ID NO:29).

FIG. 22 shows the nucleic acid sequences without and with signal sequences encoding the sequences of FIG. 21 (SEQ ID NOS:30-35).

FIG. 23 shows the sequence of the plasmid pDR1 (SEQ ID NO:45; 5391 bp) for expression of immunoglobulin light chains as described in Example 2. pDR1 contains sequences encoding an irrelevant antibody, and the light chain of a humanized anti-CD3 antibody (Shalaby et al., J. Exp. Med., 175: 217-225 (1992)), the start and stop codons for which are indicated in bold and underlined.

FIG. 24 shows the sequence of plasmid pDR2 (SEQ ID NO:46; 6135 bp) for expression of immunoglobulin heavy chains as described in Example 2. pDR2 contains sequences encoding an irrelevant antibody, and the heavy chain of a humanized anti-CD3 antibody (Shalaby et al., supra), the start and stop codons for which are indicated in bold and underlined.

### DETAILED DESCRIPTION OF THE PREFERRED EMBODIMENTS

I. Definitions

As used herein, “TGF-beta” refers to all isoforms of TGF-beta. There are currently 5 known isoforms of TGF-beta (1-5), all of which are homologous (60-80% identity) and all of which form homodimers of about 25 KD, and act upon common TGF-beta cellular receptors (Types I, II, and III). The genetic and molecular biology of TGF-beta is well known in the art (see, for example, Roberts, Miner, Electrolyte and Metab., 24(2-3):111-119 (1998); Wrana, Miner, Electrolyte and Metab., 24(2-3):120-130 (1998))

“Bioactive TGF-β1” as used herein is meant to encompass any biologically active form of TGF-β1 polypeptide, e.g., a TGF-β1 having serines substituted for the cysteines at positions 223 and 225 of the TGF-β1 pro-peptide (see Samuel et al., EMBO J., 11:1599-1605 (1992); Brunner, J. Biol. Chem., 264:13660 (1989)), or biologically active portion, isoform, homolog, variant, or analog thereof.

The term “antagonist” refers to molecules or compounds that inhibit the action of a “native” or “natural” compound. Antagonists may or may not be homologous to these natural compounds in respect to conformation, charge or other characteristics. Thus, antagonists may be recognized by the same or different receptors that are recognized by an agonist. Antagonists may have allosteric effects that prevent the action of an agonist. Or, antagonists may prevent the function of the agonist. In contrast to the agonists, antagonistic compounds do not result in physiologic and/or biochemical changes within the cell such that the cell reacts to the presence of the antagonist in the same manner as if the natural compound was present.

As used herein, the term “TGF-β antagonist” refers any agent (e.g., natural or synthetic agents, biomolecules or organic compounds, etc.) that is able to decrease the amount or activity of TGF-β, either within a cell or within a physiological system. Preferably, the TGF-beta antagonist acts to decrease the amount or activity of a TGF-β1, 2, or 3. For example, a TGF-β antagonist may be a molecule that inhibits expression of TGF-β at the level of transcription, translation, processing, or transport; it may affect the stability of TGF-β or conversion of the precursor molecule to the active, mature form; it may affect the ability of TGF-β to bind to one or more cellular receptors (e.g., Type I, II or III); or it may interfere with TGF-β signaling, as by specifically inhibiting the TGF-β signaling pathway, through inhibition of a normally TGF-β-mediated cellular response at the level of the TGF-β receptor (e.g., blocking TGF-β binding to the receptor or inhibiting induction of signaling by bound TGF-β), through interaction with a factor in the TGF-β signaling pathway, or by otherwise inhibiting the TGF-β signaling pathway to provide for a decrease in cellular response normally mediated by TGF-β.

TGF-β antagonists include antibodies directed against one or more isoforms of TGF-β such as TGF-beta1, TGF-beta2, and/or TGF-beta3, including monoclonal and polyclonal antibodies directed against one or more isoforms of TGF-β (Dasch et al., U.S. Pat. No. 5,571,714; see also, WO 97/13844 and WO 00/66631), chimeric, humanized, and human antibodies; TGF-β receptors such as dominant negative TGF-β receptors and soluble forms and fragments thereof that bind to TGF-β, especially TGF-β type II receptor (TGFBIIR) or TGF-β type III receptor (TGFBIIIR, or betaglycan) comprising, e.g., the extracellular domain of TGFBIIR or TGFBIIIR, most preferably a recombinant soluble TGF-β receptor (rsTGFBIIR or rsTGFBIIIR), all of which may be effectively introduced via gene transfer, as demonstrated herein; antibodies directed against TGF-β receptors (Segarini et al., U.S. Pat. No. 5,693,607; Lin et al., U.S. Pat. No. 6,001,969, U.S. Pat. No. 6,010,872, U.S. Pat. No. 6,086,867, U.S. Pat. No. 6,201,108; WO 98/48024; WO 95/10610; WO 93/09228; WO 92/00330); SR2F receptor antibody, antisense TGF-β DNA; latency-associated peptide (WO 91/08291); large latent TGF-β (WO 94/09812); TGF-β-inhibiting proteoglycans such as fetuin (U.S. Pat. No. 5,821,227), decorin and other proteoglycans such as biglycan, fibromodulin, lumican and endoglin (WO 91/10727; Ruoslahti et al., U.S. Pat. No. 5,654,270, U.S. Pat. No. 5,705,609, U.S. Pat. No. 5,726,149; Border, U.S. Pat. No. 5,824,655; WO 91/04748; Letarte et al., U.S. Pat. No. 5,830,847, U.S. Pat. No. 6,015,693; WO 91/10727; WO 93/09800; and WO 94/10187); somatostatin (WO 98/08529); mannose-6-phosphate or mannose-1-phosphate (Ferguson, U.S. Pat. No. 5,520,926); prolactin (WO 97/40848); insulin-like growth factor II (WO 98/17304); IP-10 (WO 97/00691); the tripeptide arg-gly-asp and peptides containing the tripeptide (Pfeffer, U.S. Pat. No. 5,958,411; WO 93/10808); TGF-α-inhibitory extracts from plants, fungi, or bacteria (EP-A-813875; JP 8119984; and Matsunaga et al., U.S. Pat. No. 5,693,610); antisense oligonucleotides, e.g., that inhibit TGF-β gene transcription or translation (Chung, U.S. Pat. No. 5,683,988; Fakhrai et al., U.S. Pat. No. 5,772,995; Dzau, U.S. Pat. No. 5,821,234, U.S. Pat. No. 5,869,462; and WO 94/25588); proteins involved in TGF-β signaling, including SMADs such as SMAD6 and SMAD7 and MADs (EP-A-874 046; WO 97/31020; WO 97/38729; WO 98/03663; WO 98/07735; WO 98/07849; WO 98/45467; WO 98/53068; WO 98/55512; WO 98/56913; WO 98/53830; WO 99/50296; Falb, U.S. Pat. No. 5,834,248; Falb et al., U.S. Pat. No. 5,807,708; and Gimeno et al., U.S. Pat. No. 5,948,639); Ski, or Sno (Vogel, Science, 286:665 (1999); and Stroschein et al., Science, 286:771-774 (1999)); any mutants, fragments or derivatives of the above-identified molecules that retain the ability to inhibit the activity of TGF-β; and small organic molecules.

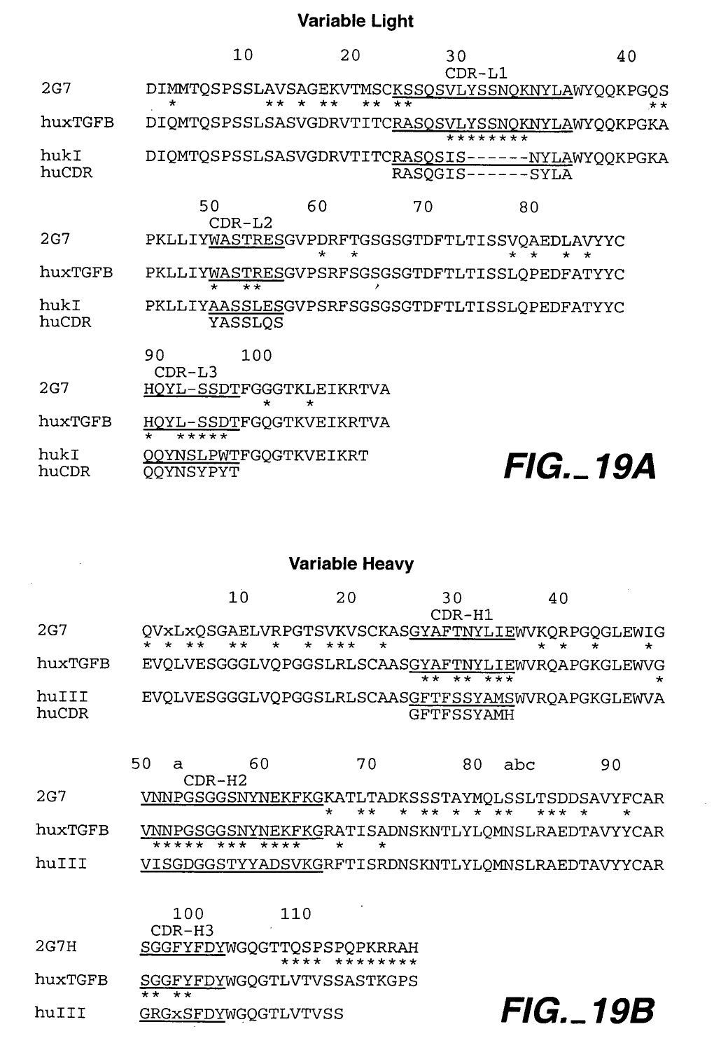

Preferably, the TGF-β antagonist is a TGF-beta1, TGF-beta2, or TGF-beta3 antagonist. More preferably, the antagonist is a TGF-beta1 antagonist. In a preferred embodiment, the TGF-β antagonist is a human monoclonal antibody that blocks TGF-β binding to its receptor, or fragments thereof such as F(ab)₂ fragments, Fv fragments, single-chain antibodies and other forms of “antibodies” that retain the ability to bind to TGF-β. In one embodiment, the TGF-β antagonist is a human antibody produced by phage display (WO 00/66631). In another preferred embodiment, the TGF-β antagonist is a human or humanized monoclonal antibody that blocks TGF-β binding to its receptor (or fragments thereof such as F(ab)₂ fragments, Fv fragments, single-chain antibodies and other forms or fragments of antibodies that retain the ability to bind to TGF-β). Preferred monoclonal antibodies are murine monoclonal antibodies 2G7 and 4A11 as described in Example 1 herein, as well as human or humanized forms thereof as set forth in Example 2 herein, and the murine monoclonal antibodies obtained from hybridoma 1 D11.16 (ATCC Accession No. HB 9849, described in Dasch et al., U.S. Pat. No. 5,783,185). More preferred are human or humanized forms of such murine antibodies, for example, those described in Example 2 herein. To screen for antibodies that bind to an epitope on TGF-beta bound by an antibody of interest, a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art (see, e.g. FIGS. 19A and 19B herein).

Suitable TGF-β antagonists for use in the present invention will also include functional mutants, variants, derivatives and analogues of the aforementioned TGF-β antagonists, so long as their ability to inhibit TGF-β amount or activity is retained. As used herein, “mutants, variants, derivatives and analogues” refer to molecules with similar shape or structure to the parent compound and that retain the ability to act as TGF-beta antagonists. For example, any of the TGF-beta antagonists disclosed herein may be crystallized, and useful analogues may be rationally designed based on the coordinates responsible for the shape of the active site(s).

Alternatively, the ordinarily skilled artisan may, without undue experimentation, modify the functional groups of a known antagonist and screen such modified molecules for increased activity, half-life, bioavailability or other desirable characteristics. Where the TGF-beta antagonist is a polypeptide, fragments and modifications of the polypeptide may be produced to increase the ease of delivery, activity, half-life, etc (for example, humanized antibodies or functional antibody fragments, as discussed above). Given the level of skill in the art of synthetic and recombinant polypeptide production, such modifications may be achieved without undue experimentation. Persons skilled in the art may also design novel inhibitors based on the crystal structure and/or knowledge of the active sites of the TGF-beta antagonists described herein.

The term “substance” is synonymous with “compound” and refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” such as a known chemotherapeutic or cytotoxic agent refers to a therapeutic compound that has been shown (e.g., through animal trials or prior experience with administration to humans) to be effective in such treatment. In other words, a known therapeutic compound is not limited to a compound efficacious in the treatment of symptoms associated with the pathological factor involved, such as TGF-β.

The term “test substance” is used herein to refer to any substance, including, without limitation, polypeptides, proteins, peptides, and small organic molecules, that is tested for a beneficial use in a screening assay or animal model of the present invention. The test substances specifically include antibodies, including murine, chimeric, humanized and human antibodies.

The term “primary tumor” is used herein to refer to a tumor that is first in order or in time of development.

The term “secondary tumor” is used herein to refer a tumor that has spread (metastasized) from the organ or location where it first appeared to another organ or another part of the body. Thus, breast cancer that has spread to the bones is not bone cancer, rather secondary (metastasized) breast cancer since the cancer cells are still breast cancer cells, regardless of their location.

The term “metastasis” is used herein to refer to the spread of cancer from one part of the body to another. The metastatic process is a sequence of steps, including invasion, intravasation, transport, arrest, extravasation, and growth, that must be accomplished by cancer cells before distant metastases are established.

The term “adverse effect on the status” of a primary tumor is used herein to refer to any effect that results in the growth of the primary tumor or the migration (spread) of primary tumor cells.

The “non-human animals” of the invention comprise any non-human animal, including vertebrates such as rodents, non-human primates, ovines, bovines, ruminants, lagomorphs, porcines, caprines, equines, canines, felines, avians, etc. Preferred non-human animals are selected from porcines (e.g., pigs) and rodents such as murines (e.g., rats and mice), most preferably rodents such as mice. However, it is not intended that the present invention be limited to any particular non-human animal.

As used herein, the term “mammal” refers to any animal categorized as a mammal, including, but not limited to, humans, non-human primates, rodents, and the like, which is to be the recipient of a particular treatment, preferably the non-human animal model herein or a human.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, melanoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. Thus, the term encompasses the improvement and/or reversal of the symptoms associated with a pathological factor such as TGF-β. “Improvement in the physiologic functions of the non-human animals of the present invention may be assessed using any of the measurements described herein, as well as any effect upon the animals' survival; the response of treated animals and untreated animals is compared using any of the assays described herein. A substance that causes an improvement in any parameter associated with a pathological factor such as TGF-β when used in the screening methods of the instant invention may thereby be identified as a therapeutic compound.

An “effective amount” or “effective dose” refers to an amount effective, at dosages and for periods of time necessary, to achieve the desired therapeutic or prophylactic result. A therapeutically effective amount” of the antibody may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), and toxins such as small-molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin γ₁^{I} and calicheamicin θ^{I}₁, see, e.g., Agnew, Chem Intl. Ed. Enql. 33:183-186 (1994); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; podophyllinic acid; 2-ethylhydrazide; procarbazine; PSK® (krestin); razoxane; rhizoxin; sizofiran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; XELODA® (capecitabine); ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are anti-hormonal agents that act to regulate or inhibit hormone action on tumors such as anti-estrogens including, for example, tamoxifen, raloxifene, aromatase inhibiting 4(5)-imidazoles, 4-hydroxytamoxifen, trioxifene, keoxifene, LY117018, onapristone, and toremifene (FARESTON®); and anti-androgens such as flutamide, nilutamide, bicalutamide, leuprolide, and goserelin; and pharmaceutically acceptable salts, acids or derivatives of any of the above.

As used herein, “taxoid” or “taxane” refers to a family of complex diterpenes present in the bark and leaves of the Pacific Yew tree (Taxus brevifolia) and derivatives thereof. Members of the taxoid or taxane family include, but are not limited to, paclitaxel (TAXOL®) and its derivatives, such as baccatin III, cephalomannine, 10-deacetylbaccatin III, 10-deacetyltaxol, 7-epi-10-deacetyltaxol, 7-xylosyl-10-deacetyltaxol, 7-epi-taxol, baccatin V, 7-epi-10-deacetyl-baccatin III, doxetaxel (TAXOTERE®), 2-debenzoyl-2-(p-trifluromethylbenzoyl)taxol, and 20-acetoxy-4-deacetyl-5-epi-20,O-secotaxol.

The term “cytokine” is a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle-stimulating hormone (FSH), thyroid-stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; a tumor necrosis factor such as TNF-αor TNF-β; and other polypeptide factors including LIF and kit ligand (KL). As used herein, the term cytokine includes proteins from natural sources or from recombinant cell culture and biologically active equivalents of the native-sequence cytokines.

A “growth-inhibitory agent” when used herein refers to a compound or composition that inhibits growth of a cell, especially a TGF-beta-expressing cancer cell either in vitro or in vivo. Thus, the growth-inhibitory agent may be one that significantly reduces the percentage of TGF-beta-expressing cells in S phase. Examples of growth-inhibitory agents include agents that block cell-cycle progression (at a place other than S phase), such as agents that induce G1 arrest and M-phase arrest. Classical M-phase blockers include the vincas (vincristine and vinblastine), taxanes, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

Examples of “growth-inhibitory” antibodies are those that bind to TGF-beta and inhibit the growth of cancer cells overexpressing TGF-beta. Preferred growth-inhibitory anti-TGF-beta antibodies inhibit growth of SK-BR-3 breast tumor cells in cell culture by greater than 20%, and preferably greater than 50% (e.g. from about 50% to about 100%) at an antibody concentration of about 0.5 to 30 μg/ml, where the growth inhibition is determined six days after exposure of the SK-BR-3 cells to the antibody (see U.S. Pat. No. 5,677,171 issued Oct. 14, 1997). The SK-BR-3 cell-growth inhibition assay is described in more detail in that patent and hereinbelow.

An antibody that “induces cell death” is one that causes a viable cell to become nonviable. The cell is generally one that expresses the TGF-beta receptor, especially where the cell overexpresses the TGF-beta receptor. Preferably, the cell is a cancer cell, e.g. a breast, ovarian, stomach, endometrial, salivary gland, lung, kidney, colon, thyroid, pancreatic or bladder cell. In vitro, the cell may be a SK-BR-3, BT474, Calu 3, MDA-MB-453, MDA-MB-361 or SKOV3 cell. Cell death in vitro may be determined in the absence of complement and immune effector cells to distinguish cell death induced by antibody-dependent cell-mediated cytotoxicity (ADCC) or complement-dependent cytotoxicity (CDC). Thus, the assay for cell death may be performed using heat-inactivated serum (i.e. in the absence of complement) and in the absence of immune effector cells. To determine whether the antibody is able to induce cell death, loss of membrane integrity as evaluated by uptake of propidium iodide (PI), trypan blue (see Moore et al., Cytotechnology, 17:1-11 (1995)) or 7MD can be assessed relative to untreated cells. Preferred cell-death-inducing antibodies are those that induce PI uptake in the PI uptake assay in BT474 cells (see below).

An antibody that “induces apoptosis” is one that induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one that overexpresses the TGF-beta receptor. Preferably the cell is a tumor cell, e.g., a breast, ovarian, stomach, endometrial, salivary gland, lung, kidney, colon, thyroid, pancreatic or bladder cell. In vitro, the cell may be a SK− BR-3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody that induces apoptosis is one that results in about 2 to 50 fold, preferably about 5 to 50 fold, and most preferably about 10 to 50 fold, induction of annexin binding relative to untreated cell in an annexin binding assay using BT474 cells (see below). Sometimes the pro-apoptotic antibody will be one that further blocks TGF-beta binding (e.g. 2G7 antibody); i.e. the antibody shares a biological characteristic with an antibody to TGF-beta. In other situations, the antibody is one that does not significantly block TGF-beta. Further, the antibody may be one that, while inducing apoptosis, does not induce a large reduction in the percent of cells in S phase (e.g. one that only induces about 0-10% reduction in the percent of these cells relative to control).

The term “antibody” is used in the broadest sense and includes monoclonal antibodies, polyclonal antibodies, multivalent antibodies, multispecific antibodies (e.g., bispecific antibodies), full-length antibodies, and antibody fragments so long as they exhibit the desired biological activity. A naturally occurring antibody comprises four polypeptide chains, two identical heavy (H) chains and two identical light (L) chains inter-connected by disulfide bonds. Each heavy chain is comprised of a heavy-chain variable region (V_{H}) and a heavy-chain constant region, which in its native form is comprised of three domains, CH1, CH2 and CH3. Each light chain is comprised of a light-chain variable region (V_{L}) and a light-chain constant region. The light-chain constant region is comprised of one domain, C_{L}. The V_{H} and V_{L} regions can be further subdivided into regions of hypervariability, termed complementarity-determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgA-1, IgA-2, etc. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al., Cellular and Mol. Immunology, 4th ed. (2000). The light chains of antibodies from any vertebrate species can be assigned to one of two clearly distinct types, called kappa (κ) and lambda (λ), based on the amino acid sequences of their constant domains. Preferably, the antibody herein is an immunoglobulin G, more preferably, a human immunoglobulin G.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) or Marks et al., J. Mol. Biol., 222:581-597 (1991), for example.

“Full-length antibody” refers to an intact antibody as would be found in nature and is not a fragment.

“Antibody fragments” comprise only a portion of an intact antibody, generally including an antigen-binding site of the intact antibody and thus retaining the ability to bind antigen. Examples of antibody fragments encompassed by the present definition include: (i) the Fab fragment, having VL, CL, VH and CH1 domains, i.e., containing both variable regions and the constant domain of the light chain and the first constant domain (CH1) of the heavy chain; (ii) the Fab′ fragment, which differs from Fab fragments by the addition of a few residues at the carboxyl terminus of the heavy-chain CH1 domain, including one or more cysteine(s) from the antibody hinge region; (iii) the Fab′-SH fragment, which is a Fab′ fragment in which the cysteine residue(s) of the constant domains bear a free thiol group;
(iv) the Fv fragment having the VL and VH domains of a single arm of an antibody; (v) the F(ab′)₂ fragment, a bivalent fragment including two Fab′ fragments linked by a disulphide bridge at the hinge region; (vi) single-chain antibody molecules (e.g. single chain Fv; scFv) (Bird et al., Science, 242:423-426 (1988); and Huston et al., Proc. Natl. Acad. Sci. USA, 85:5879-5883 (1988)); and (vii) “diabodies” with two antigen-binding sites, comprising a heavy-chain variable domain (VH) connected to a light-chain variable domain (VL) in the same polypeptide chain (see, e.g., EP 404,097; WO 93/11161; and Hollinger et al., Proc. Natl. Acad. Sci. USA, 90:6444-6448 (1993)).

An antibody or region thereof with a “native sequence” or a “native-sequence” antibody or region thereof refers to an antibody or region thereof having the same amino acid sequence as the corresponding portion of an antibody derived from nature. Thus, an antibody with a native sequence can have the amino acid sequence of that corresponding antibody of naturally occurring antibody from any mammal. Such antibody with native sequence can be derived from an antibody isolated from nature or produced by recombinant or synthetic means.

A variant antibody or region thereof means a biologically active antibody or region thereof having at least about 80% amino acid sequence identity with the corresponding antibody or region thereof with a native sequence. Such variants include, for instance, full-length antibodies and antibody fragments or light-chain or heavy-chain regions thereof wherein one or more amino acid residues are added, or deleted, at the N- or C-terminus of the antibody or fragment or region or within the antibody, fragment, or region. Ordinarily, a variant will have at least about 80% amino acid sequence identity, more preferably at least about 90% amino acid sequence identity, and even more preferably at least about 95% amino acid sequence identity with the corresponding antibody or region thereof with a native sequence.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program, authored by Genentech, Inc., has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

A “functional” or “biologically active” antibody is one capable of exerting one or more of its natural activities in structural, regulatory, biochemical, or biophysical events. For example, a functional antibody may have the ability to specifically bind an antigen and the binding may in turn elicit or alter a cellular or molecular event such as signaling transduction or enzymatic activity. A functional antibody may also block ligand activation of a receptor or act as an agonist antibody. The capability of an antibody to exert one or more of its natural activities depends on several factors, including proper folding and assembly of the polypeptide chains. As used herein, the functional antibodies generated by the disclosed methods typically have two identical L chains and two identical H chains that are linked by multiple disulfide bonds and properly folded.

Unless indicated otherwise, the expression “multivalent antibody” is used throughout this specification to denote an antibody comprising three or more antigen-binding sites. The multivalent antibody is preferably engineered to have the three or more antigen-binding sites and is generally not a native-sequence IgM or IgA antibody.

The antibody herein specifically includes “chimeric” antibody in which a portion of the heavy and/or light chain is identical with or homologous to corresponding sequences in antibody derived from a particular species or belonging to a particular antibody class or subclass, while the remainder of the chain(s) is identical with or homologous to corresponding sequences in antibody derived from another species or belonging to another antibody class or subclass, so long as they exhibit the desired biological activity (U.S. Pat. No. 4,816,567; and Morrison et al., Proc. Natl. Acad. Sci. USA, 81:6851-6855 (1984)).

“Humanized” antibody is chimeric antibody that contains minimal sequence derived from non-human immunoglobulin. For the most part, humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibody may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature, 321:522-525 (1986); Riechmann et al., Nature, 332:323-329 (1988); and Presta, Curr. Op. Struct. Biol., 2:593-596 (1992).

A “human antibody” is one that possesses an amino acid sequence corresponding to that of an antibody produced by a human and/or has been made using any of the techniques for making human antibody as disclosed herein. This definition of a human antibody specifically excludes a humanized antibody comprising non-human antigen-binding residues. Human antibody can be produced using various techniques known in the art. In one embodiment, the human antibody is selected from a phage library, where that phage library expresses human antibody (Vaughan et al., Nature Biotechnology, 14:309-314 (1996): Sheets et al., PNAS (USA), 95:6157-6162 (1998)); Hoogenboom and Winter, J. Mol. Biol., 227:381 (1991); Marks et al., J. Mol. Biol., 222:581 (1991)). Human antibody can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology, 10: 779-783 (1992); Lonberg et al., Nature, 368: 856-859 (1994); Morrison, Nature, 368:812-813 (1994); Fishwild et al., Nature Biotechnology, 14: 845-51 (1996); Neuberger, Nature Biotechnology, 14: 826 (1996); Lonberg and Huszar, Intern. Rev. Immunol., 13:65-93 (1995). Alternatively, the human antibody may be prepared via immortalization of human B-lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147(1):86-95 (1991); and U.S. Pat. No. 5,750,373.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

An “affinity-matured” antibody is one with one or more alterations in one or more CDRs thereof that result in an improvement in the affinity of the antibody for antigen, compared to a corresponding parent antibody that does not possess those alteration(s). Preferred affinity-matured antibodies will have nanomolar or even picomolar affinities for the target antigen. Affinity-matured antibodies are produced by procedures known in the art. Marks et al., Bio/Technology, 10:779-783 (1992) describes affinity maturation by VH and VL domain shuffling. Random mutagenesis of CDR and/or framework residues is described by: Barbas et al., Proc. Nat. Acad. Sci, USA, 91:3809-3813 (1994); Schier et al., Gene, 169:147-155 (1995); Yelton et al., J. Immunol., 155:1994-2004 (1995); Jackson et al, J. Immunol., 154(7): 3310-3319 (1995); and Hawkins et al, J. Mol. Biol., 226:889-896 (1992).

An “isolated” or “recovered” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of polypeptide as determined by the Lowry method, and most preferably more than 99% by weight, (2) to a degree sufficient to obtain at least 15 residues of N-terminal or internal amino acid sequence by use of a spinning-cup sequenator, or (3) to homogeneity by SDS-PAGE under reducing or nonreducing conditions using Coomassie blue or, preferably, silver stain. Isolated or recovered antibody includes the antibody in situ within recombinant cells since at least one component of the natural environment of the antibody will not be present. Ordinarily, however, isolated or recovered antibody will be prepared by at least one purification step.

The term “antigen” is well understood in the art and includes substances that are immunogenic, i.e., immunogens, as well as substances that induce immunological unresponsiveness, or anergy, i.e., anergens. Where the antigen is a polypeptide, it may be a transmembrane molecule (e.g. receptor) or ligand such as a growth factor. Exemplary antigens include molecules such as renin; a growth hormone, including human growth hormone and bovine growth hormone; growth-hormone releasing factor; parathyroid hormone; thyroid-stimulating hormone; lipoproteins; alpha-1-antitrypsin; insulin A-chain; insulin B-chain; proinsulin; follicle-stimulating hormone; calcitonin; luteinizing hormone; glucagon; clotting factors such as factor VIIIC, factor IX, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)—IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony-stimulating factors (CSFs), e.g., M-CSF, GM-CSF, and G-CSF; interleukins (ILs), e.g., IL-1 to IL-10; superoxide dismutase; T-cell receptors; surface-membrane proteins; decay-accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor-associated antigen such as HER2, HER3 or HER4 receptor; and fragments of any of the above-listed polypeptides.

Preferred antigens for which the antibodies used in the method of the present invention are specific or are directed to are TGF-β1, TGF-β2, TGF-β3, TGF-β4, TGF-β5, IFN-γ, FGF, EGF, as well as receptors of the native TGF-β polypeptides, such as TGFβ-RI and TGFβ-RII. Other preferred antigens are antigens present in the TGF-β signaling pathway, such as, for example, Smad2, Smad3, Smad2/3, Smad 4, Smad 7, JNK, p38 MAPK, erk MAPK, TAK1/MEKK1, Ras, RhoA, PP2A, MKK3/6, MKK4, p160Rock, and S6K.

Throughout the disclosure, the terms “ErbB2”, “ErbB2 receptor”, “c-erb-B2”, “HER2,” and “Her2” are used interchangeably, and, unless otherwise indicated, refer to a native-sequence ErbB2 human polypeptide, or a functional derivative thereof. “Her2”, “erbB2” and “c-erb-B2” refer to the corresponding human gene. The terms “native-sequence” or “native” in this context refer to a polypeptide having the sequence of a naturally occurring polypeptide, regardless of its mode of preparation. Such native-sequence polypeptides can be isolated from nature or can be produced by recombinant or synthetic means, or by any combination of these or similar methods.

Humanized anti-ErbB2 antibodies include huMAb4D5-1, huMAb4D5-2, huMAb4D5-3, huMAb4D5-4, huMAb4D5-5, huMAb4D5-6, huMAb4D5-7 and huMAb4D5-8 (trastuzumab (HERCEPTIN®)) as described in Table 3 of U.S. Pat. No. 5,821,337 expressly incorporated herein by reference; humanized 520C9 (WO93/21319) and humanized 2C4 antibodies.

The terms “Her2-expressing cancer (tumor)” and “Her2+cancer (tumor)” are used interchangeably, and refer to cancer (tumor) comprising cells which have Her2 protein present at their cell surface. A “Her2-expressing cancer” is one that produces sufficient levels of Her2 at the surface of cells thereof, such that an anti-Her2 antibody can bind thereto and have a therapeutic effect with respect to the cancer. A Her2′ or Her2-negative cancer (tumor) is a tumor comprising cells that do not have Her2 protein present at their cell surface.

A “trastuzumab-resistant tumor” does not show statistically significant improvement in response to trastuzumab (HERCEPTIN®) treatment when compared to no treatment or treatment with placebo in a recognized animal model or a human clinical trial, or which responds to initial treatment with trastuzumab but grows as treatment is continued. In contrast, a “trastuzumab-respondent” or “trastuzumab-sensitive” tumor does show statistically significant improvement in response to trastuzumab treatment when compared to no treatment or treatment with placebo in a recognized animal model or a human clinical trial.

Unless indicated otherwise, the expression “monoclonal antibody 2G7” refers to an antibody that has antigen-binding residues of, or derived from, the murine 2G7 antibody of the Examples below. For example, the monoclonal antibody 2G7 may be murine monoclonal antibody 2G7 or a variant thereof, such as a humanized antibody 2G7, possessing antigen-binding amino acid residues of murine monoclonal antibody 2G7.

Example 2 below describes production of exemplary humanized anti-TGF-beta antibodies that bind TGF-beta. The humanized antibody herein comprises non-human hypervariable region residues incorporated into a human variable heavy domain and further comprises a framework region (FR) substitution at a position selected from the group consisting of 48, 49, 67, 69, 71, 73, and 78, utilizing the variable-domain numbering system set forth in Kabat et al., supra. In one embodiment, the humanized antibody comprises FR substitutions at two or more of positions 48, 49, 67, 69, 71, 73, and 78; and in other embodiments, at three or four or more of such positions. In preferred embodiments, the antibody comprises FR substitutions at positions 49, 67 and 71, positions 48, 49 and 71, or positions 49, 69, and 71, or positions 49, 69, 71, and 73, or positions 49, 71, and 73, or at positions 49, 71, and 78. It is preferred that there are fewer rather than more framework substitutions to minimize immunogenicity, but efficacy is also a very important consideration. The amino acids actually substituted are those that are preferably conserved so as not to change the immunogenicity or efficacy. At position 48, the change is preferably from valine to isoleucine, at position 49, the change is preferably from alanine to glycine, at position 67, the change is preferably phenylalanine to alanine, at position 69, the change is preferably phenylalanine to alanine, at position 71, the change is preferably arginine to alanine, at position 73, the change is preferably asparagine to lysine, and at position 78, the change is preferably leucine to alanine.

An exemplary humanized antibody of interest herein comprises variable heavy-domain complementarity-determining residues GYAFTNYLIE (SEQ ID NO:41); VNNPGSGGSNYNEKFKG (SEQ ID NO:42) or VINPGSGGSNYNEKFKG (SEQ ID NO:43); and/or SGGFYFDY (SEQ ID NO-44), optionally comprising amino acid modifications of those CDR residues, e.g. where the modifications essentially maintain or improve affinity of the antibody. For example, the antibody variant of interest may have from about one to about seven or about five amino acid substitutions in the above variable heavy-domain CDR sequences. Such antibody variants may be prepared by affinity maturation, e.g., as described below. Preferably, the residues are two or more of GYAFTNYLIE (SEQ ID NO:41); VNNPGSGGSNYNEKFKG (SEQ ID NO:42) or VINPGSGGSNYNEKFKG (SEQ ID NO:43); and/or SGGFYFDY (SEQ ID NO:44), most preferably all three. The most preferred humanized antibody comprises the variable heavy-domain amino acid sequence in SEQ ID NO:4 or the one with GYAFTNYLIE (SEQ ID NO:41); VINPGSGGSNYNEKFKG (SEQ ID NO:43); and SGGFYFDY (SEQ ID NO:44).

The humanized antibody may comprise variable light-domain complementarity-determining residues RASQSVLYSSNQKNYLA (SEQ ID NO:36) or RASQGISSYLA (SEQ ID NO:37); WASTRES (SEQ ID NO:38) or YASSLQS (SEQ ID NO:39); and/or HQYLSSDT (SEQ ID NO:40), e.g. in addition to those variable heavy-domain CDR residues in the preceding paragraph. Such humanized antibodies optionally comprise amino acid modifications of the above CDR residues, e.g. where the modifications essentially maintain or improve affinity of the antibody. For example, the antibody variant of interest may have from about one to about seven or about five amino acid substitutions in the above variable light CDR sequences. Such antibody variants may be prepared by affinity maturation, e.g., as described below. Preferably, the residues are two or more of RASQSVLYSSNQKNYLA (SEQ ID NO:36); WASTRES (SEQ ID NO:38); and/or HQYLSSDT (SEQ ID NO:40), most preferably all three. The most preferred humanized antibody comprises the variable light domain amino acid sequence in SEQ ID NO:3.

The present application also contemplates affinity-matured antibodies that bind TGF-beta. The parent antibody may be a human antibody or a humanized antibody, e.g., one comprising the variable light and/or heavy sequences of SEQ ID Nos. 3 and 4, respectively (i.e. Version 5). The affinity-matured antibody preferably binds to TGF-beta with an affinity superior to that of murine 2G7 or variant 5 (e.g. from about two or about four fold, to about 100 fold or about 1000 fold improved affinity, e.g. as assessed using a TGF-beta-extracellular domain (ECD) ELISA).

A patient “predetermined not to respond, or to respond poorly, to treatment with a TGF-beta antagonist” does not show statistically significant improvement in response to treatment with a TGF-beta antagonist when compared to no treatment or treatment with placebo when testing the responsiveness of the patient's tumor in a recognized in vitro or animal model or a human clinical trial, or where the patient responds to initial treatment with a TGF-beta antagonist but the response is transient, and the tumor grows as treatment is continued.

An “anti-angiogenic agent” refers to a compound other than a TGF-beta antagonist that blocks, or interferes with, to some degree, the development of blood vessels. The anti-angiogenic factor may, for instance, be a small molecule or antibody that binds to a growth factor or growth factor receptor involved in promoting angiogenesis. An example is an antagonist to vascular endothelial growth factor (VEGF), such as an antibody that specifically binds VEGF, such as bevacizumab (AVASTIN®).

II. Modes for Carrying out the Invention

As discussed earlier, TGF-β plays a complex role in carcinogenesis. The TGF-β pathway acts as a tumor suppressor in early stages of epithelial cell carcinogenesis. With changes in the genetic and epigenetic context of pre-cancerous and cancerous cells, the TGF-β responsiveness of cells declines, and increased TGF-β expression/activation is observed until in late, pre-metastatic stages of tumor development and in invasive metastatic cancer the pro-oncogenic role of the TGF-β pathway becomes predominant. For further details see Roberts and Wakefield, Proc. Natl. Acad. Sci. USA, 100(15):8621-8623 (2003). It is known that some tumors, such as various carcinomas, evade the inhibition of cell growth by TGF-β as a result of inactivating mutations in the TGF-β receptors. The fact that TGF-β (and other members of the TGF-β pathway) can act directly as a tumor promoter is supported by the fact that many tumors do not have inactivated TGF-β receptors; therefore, the formation and spread of such tumors cannot be explained by the evasion of TGF-β inhibition of cell growth as a result of inactivating mutations.

In a number of tumor cell model systems, pretreatment with purified TGF-β or transfection with TGF-β1 cDNA results in an increase in metastatic potential. Conversely, blocking the tumor cell responsiveness to TGF-β or neutralizing TGF-β production decreases metastatic efficiency in vivo. This strongly suggests that TGF-β can promote metastasis. Possible mechanisms for which evidence has been obtained include: (i) suppression of immune surveillance; (ii) promotion of invasiveness and motility; and (iii) promotion of angiogenesis. However, an understanding of the mechanisms is not necessary in order to use the present invention. Indeed, it is not intended that the present invention be limited to any particular mechanism(s).

The present invention is based on experimental data obtained by testing anti-TGF-β antibodies in several animal models, including those produced by using cell lines from spontaneous tumors as well as by using primary cells prepared from oncogene-driven tumors. Similarly to the heterogeneity observed in human tumors, animal models show varied responses to treatment with TGF-β antagonists, such an anti-TGF-β antibodies. The information generated in these animal models allows differentiation between the various TGF-β-induced activities on tumor cells, and has important implications for identifying substances for the preferential treatment of a particular type, stage or form of cancer, such as secondary (metastatic) tumors, breast cancer vs. other types of cancer, various subtypes of breast cancer and the like. As a result, the experimental data underlying the present invention provide important information for personalizing cancer therapy of human patients. Since metastatic cancer is the major cause of death for patients with solid tumors, one aspect of the invention focuses on identifying substances that are effective in the treatment of secondary tumors.

Accordingly, in one embodiment, the present invention is a screening method of a substance having therapeutic activity for cancer, which comprises the following steps: (1) administering a plurality of test substances to a non-human syngeneic immunocompetent animal model bearing at least one soft tissue or bone metastasis, in the presence or absence of a primary tumor; (2) determining the effects of said test substances on the soft tissue or bone metastasis and growth of the primary tumor, if present; and (3) identifying a test substance that inhibits the growth of a soft tissue or bone metastasis, without adverse effect on the status of the primary tumor, if present.

In a variation of this method, the administration of the test substances is combined with other standard therapies for the treatment of cancer, in particular metastatic cancer, such as, for example radiation therapy.

In one embodiment, the test substances administered to said animal include a known chemotherapeutic or cytotoxic agent such as a taxoid. In a preferred aspect of this method, the animal is administered two test substances, one of which is a TGF-beta antagonist, and the other one the chemotherapeutic or cytotoxic agent, and the combined effects of the two test substances on soft tissue or bone metastasis and primary tumor growth, if primary tumor is present, are determined. In a more preferred embodiment, the TGF-beta antagonist is an antibody specifically binding TGF-beta and the chemotherapeutic or cytotoxic agent is a taxoid.

The animal used in this in vivo screening assay may be any kind of animal except human, but preferable examples of the animal include rodents, such as mice and rats, rabbits, miniature pigs, and pigs, more preferably mice.

Some animal models useful in the present invention show pathologies specific to late-stage, metastasized cancer such as breast cancer or melanoma, and therefore can be used to identify substances, e.g. chemotherapeutic and/or cytotoxic agents that offer benefits in the treatment of such aggressive, late-stage cancer, including treatment of soft tissue and bone metastases.

In order to produce animal models of tumor metastasis the injection of tumor cells into the animals must result in the formation of both primary and secondary tumors with a reproducible timing pattern of the appearance of the primary and secondary tumors; the system must be syngeneic; and the secondary tumors must be true metastases, i.e. must be formed of the cells of the primary tumor. In addition, it should be possible to culture the tumor cells used for injection in vitro, and to attain a reasonable transfection efficiency.

Transgenic animals carrying transforming genes under the control of viral promoters provide animals with spontaneously developing primary tumors. However, such animals typically die from massive primary tumors rather than disseminating tumor cells to form secondary tumors, and are, therefore, not an optimal model for the study of metastatic cancer. They can, however, serve as a source of tumor cells for injection into another animal in order to develop an appropriate animal model.

Thus, the BALB/c-derived transplantable 4T1 mouse mammary carcinoma is an established model for study of metastatic cancer. See, e.g. Aslakson and Miller, Cancer Res., 52:1399-1405 (1992); Pulaski and Ostrand-Rosenberg, Cancer Res., 58: 1486-1493 (1998); and Pulasky et al., Cancer Res., 60: 2710-2715 (2000). After inoculation of the 4T1 tumor cells into the mammary fat pad of the recipient mouse, the primary tumor growth progressively and spontaneously metastasizes to the lungs, liver and other soft tissues, and to the bones. Similarly to human breast cancer, in particular, aggressive adenocarcinoma, metastatic cells proliferate at distant sites in the presence of the primary tumor, and continue to proliferate after the primary tumor is surgically removed. Therefore, the 4T1 model is suitable for studying tumor metastasis both in the presence of and after surgical removal of the primary tumor.

In order to study the effect of various test substances on Her-2/neu expressing metastatic breast cancer, Her-2/neu overexpressing human breast cancer cells can be inoculated into the mammary fat pad of recipient mice, and treated with the test substance. Alternatively, the tumor can be transplanted into the recipient mice. This model system allows the study of both trastuzumab-resistant and trastuzumab-respondent (trastuzumab-sensitive) breast cancer. Another animal model particularly suitable for testing agents for the treatment of trastuzumab-resistant breast cancer is described in U.S. Pat. No. 6,632,979, issued Oct. 14, 2003, the entire disclosure of which is hereby expressly incorporated by reference.

Another animal model suitable for studying tumor progression and metastasis is the mouse model of breast cancer caused by expression of the polyoma middle T oncoprotein (PyMT) in the mammary epithelium. The PyMT tumors are histologically different from Her-2⁺ tumors, and undergo clearly identifiable, distinct stages of tumor development from pre-malignant or malignant stage to metastasis that occurs with high frequency. The PyMT tumors show morphological similarities with certain aggressive forms of human breast cancer associated with poor prognosis, and therefore, provide an excellent model for studying and identifying drug candidates for the treatment of such cancer. See, e.g. Lin et al., Am J. Pathol., 163(5):2113-2126 (2003).

For the discussion of further animal models of metastatic breast cancer see, e.g. Heppner et al., Breast Cancer Res. 2(5):331-334 (2000).

Metastatic melanoma can be studied, for example, in a sub-strain of Sinclair miniature swine (Sinclair Research Center, Inc.), which develops an aggressive form of melanoma very similar to the human counterpart. This aggressive melanoma has the unique characteristic of spontaneously regressing after a complete metastatic phase, and is therefore, uniquely suited for the study of the development and regression of metastatic melanoma.

In addition, the mouse melanoma cell lines B16, K1735 and Cloudman S91-M3 (and various sublines) are frequently used in the development of melanoma models. For further details of animals models suitable for the study of metastases in melanoma see, e.g. Gattoni-Celli et al., Pigment Cell Res., 6(6):38-34 (1993) and Rusciano et al., Invasion Metastasis, 14(1-6):349-361 (1994-95).

The animal models of the present invention may be used to screen substances useful for the prophylaxis or treatment of soft tissue and/or bone metastases, which may additionally be effective in treating the primary tumor. Screening for a useful drug involves administering the test substance over a range of doses to the animal model, and assaying at various time points for the effect(s) of the substance on the status of the secondary and primary tumors present.

In one embodiment, test substances are screened by being administered to the animal over a range of doses, and evaluating the animal's physiological response to the compounds over time. Administration may be oral, or by suitable injection, depending on the chemical nature of the compound being evaluated. In some cases, it may be appropriate to administer the compound in conjunction with co-factors that would enhance the efficacy of the compound.

In addition to screening a drug for use in treating a disease or condition, the methods of the present invention are also useful in studying the efficacy or mechanism of action of a particular drug, and/or designing a therapeutic regimen aimed at preventing or curing the disease or condition. For example, the animal may be treated with a combination of a particular diet, exercise routine, radiation treatment, chemotherapy and/or one or more compounds identified herein either prior to, simultaneously, or after the onset of the disease or condition. Such an overall therapy or regimen might be more effective at combating the disease or condition than treatment with a compound alone.

The screen using the transgenic animals of the invention can employ any phenomena associated with cancer that can be readily assessed in an animal model. The individual effects of a test substance on primary tumor growth and soft tissue and bone metastases can be monitored by techniques well known in the art, including primary and secondary end points. For example, the effect of a test substance on a primary or secondary tumor can be monitored by measuring tumor size, tumor incidence (number) and tropism (site), measuring endogenous TGF-β production by the tumor cells before, during and/or after treatment with the test substance, determining serum TGF-β levels before, during and/or after treatment with a test substance, histology scoring and various imaging techniques, including micro-computed tomography (micro-CT; μCT) imaging. Since in soft tissues small metastatic tumors are hard to detect and quantitate without a time-consuming process of preparing and individually examining a large number of tissue sections, micro-CT is particularly useful for such metastases as well as metastases of the bone.

Micro-CT (x-ray microtomography) is a non-destructive technique, used to create 2D and 3D X-ray attenuation maps of specimens of a few millimeters in size. In order to image lungs ex vivo, using the micro-CT technique, the lungs can be soaked in ISOVIEW™ reagent (CT contrast agent, iodine sugar). This is followed by slow infusion of soybean oil to remove the contrast agent from the airways. Images can be generated at various resolutions. Thus, most images provided herein have been generated at 16-μ resolution. This technique is compatible with histology, and the three-dimensional visualization software allows the reader to accept or reject masses as possible tumors.

Another imaging technique, which can be performed in vivo, relies on bioluminescence imaging of luciferase activity. In vivo bioluminescence is a well-known and widely used imaging technique. This technology allows the non-invasive imaging and quantification of cells expressing luciferase proteins. The major luciferase used in this assay is from the firefly, phytonis pyralis. This enzyme has a short half-life in vitro (approximately 3 minutes at 37° C.) and in vivo (approximately 90 min). Mutants with longer half lives are also commercially available. For in vivo imaging of tumors, tumor cells, such as mammary tumor cells are transfected with luciferase, and implanted into a recipient animal, e.g. mouse. Following implantation, allowing sufficient time for tumor formation, luciferin is injected into the tumor-bearing animal, e.g. mouse, intraperitoneally. The bioluminescence, produced by the reaction of luciferin, ATP and oxygen in the presence of the luciferase enzyme can be photographed by a CCD camera.

For the description of in vivo imaging of metastatic cancer with fluorescent proteins see, e.g. Hoffman, Cell Death and Differentiation, 9:786-789 (2002).

The test substance is not particularly limited, but examples thereof include polypeptides, proteins, peptides, non-peptide small organic molecules, synthetic compounds, fermented products and cell extracts.

Candidate substances encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules, including, but not limited to: peptides, saccharides, fatty acids, steroids, purines, pyrimidines, derivatives, structural analogs or combinations thereof.

Candidate substances are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate substances specifically include, without limitation, antibodies, such as, for example anti-TGF-β antibodies.

Several TGF-β1 sequences have been isolated, cloned, and sequenced. A list of TGF-β1 sequences is provided that may be suitable for use, e.g. to produce TGF-β1 antagonists, in practicing the present invention, as well as Genbank accession numbers relating to such sequences:
Human TGF-β1 AA459172
Bovine TGF-β1 M36271
precursor Human TGF-β1 E00973; X02812;
Sheep (Ovis) X76916; J05114; M38449;
TGF-β1 L36038 M55656
Porcine TGF-β1 M23703; X12373
Canine TGF-β1 L34956
Hamster TGF-β1×60296
Rat TGF-β1X52498
Murine TGF-β1 M13177

The antibody herein may be monospecific, bispecific, or trispecific or have greater multispecificity. Multispecific antibodies may be specific to different epitopes of a single molecule (e.g., F(ab′)₂ bispecific antibodies) or may be specific to epitopes on different molecules. Methods for designing and making multispecific antibodies are known in the art. See, e.g., Millstein et al., Nature, 305:537-539 (1983); Kostelny et al., J. Immunol., 148:1547-1553 (1992); and WO 93/17715. Trispecific antibodies can be prepared as described in Tutt et al., J. Immunol., 147:60 (1991).

In particular, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes. In yet a further embodiment, Fab′-SH fragments directly recovered from E. coli can be chemically coupled in vitro to form bispecific antibodies. Shalaby et al., J. Exp. Med., 175:217-225 (1992).

Various techniques for making and isolating bispecific antibody directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The udiabodyu technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Alternatively, the bispecific antibody may be a “linear antibody” produced as described in Zapata et al., Protein Eng., 8(10):1057-1062 (1995).

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques.

Other modifications of the antibody are contemplated. For example, it may be desirable to modify the antibody with respect to effector function, so as to enhance the effectiveness of the antibody in treating cancer, for example. For example, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176:1191-1195 (1992) and Shopes, J. Immunol., 148:2918-2922 (1992). Homodimeric antibodies with enhanced anti-tumor activity may also be prepared using heterobifunctional cross-linkers as described in Wolff et al., Cancer Research, 53:2560-2565 (1993).

Various techniques have been developed for the production of antibodies. Traditionally, the antibody fragments were derived via proteolytic digestion of intact antibodies (see, e.g., Morimoto et al., Journal of Biochemical and Biophysical Methods, 24:107-117 (1992) and Brennan et al., Science, 229:81 (1985)). However, these fragments as well as the full-length antibodies and other antibodies can now be produced directly by recombinant host cells, wherein DNA sequences encoding the light and heavy chains of the antibody are obtained using standard recombinant DNA techniques. Desired DNA sequences may be isolated and sequenced from antibody-producing cells such as hybridoma cells. Alternatively, the DNA can be synthesized using nucleotide synthesizer or PCR techniques. Once obtained, DNAs encoding the light and heavy chains are inserted into a recombinant vector capable of replicating, expressing and secreting heterologous polynucleotides in prokaryotic or eukaryotic hosts. For example, Fab′-SH fragments can be directly recovered from E. coli and chemically coupled to form F(ab′)₂ fragments (Carter et al., Bio/Technology, 10:163-167 (1992)). In another embodiment, the F(ab′)₂ is formed using the leucine zipper GCN4 to promote assembly of the F(ab′)₂ molecule. According to another approach, the full-length antibodies or Fab or F(ab′)₂ fragments or other antibodies can be isolated directly from recombinant host cell culture. Many vectors that are available and known in the art can be used for the purpose of the present invention. Selection of an appropriate vector will depend mainly on the size of nucleic acids to be inserted and the particular host cell to be transformed with the vector.

In general, recombinant vectors containing replicon and control sequences that are derived from species compatible with the host cell are used as parent vectors for the construction of the specific vectors of the present invention. The vector ordinarily carries as backbone components an origin of replication site as well as marking sequences that are capable of providing phenotypic selection in transformed cells. The origin of replication site is a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria.

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. One example of a selection scheme utilizes a drug to arrest growth of a host cell. Those cells that are successfully transformed with a heterologous gene produce a protein conferring drug resistance and thus survive the selection regimen. An example of plasmid vector suitable for E. coli transformation is pBR322. pBR322 contains genes encoding ampicillin (Amp) and tetracycline (Tet) resistance and thus provides easy means for identifying transformed cells. Derivatives of pBR322 or other microbial plasmids or bacteriophage may also be used as parent vectors. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as λGEM.TM.-11 may be utilized in making a recombinant vector that can be used to transform susceptible host cells such as E. coli LE392.

In one preferred embodiment, the process temporally separates the expression of light-chain and heavy-chain portions of the antibody. In particular, the process preferably comprises transforming the host cell with two separate translational units respectively encoding the light and heavy chains; culturing the cell under suitable conditions such that the light chain and heavy chain are expressed in a sequential fashion, thereby temporally separating the production of the light and heavy chains; and allowing the light and heavy chains to assemble into the functional antibody.

In one preferred aspect of this embodiment, the temporally separated expression of light and heavy chains is realized by utilizing two different promoters separately controlling the light and heavy chains, wherein the different promoters are activated under different conditions. For example, DNAs encoding the light and heavy chains can be incorporated into a single plasmid vector but are separated into two translational units, each of which is controlled by a different promoter. One promoter (for example, a first promoter) can be either constitutive or inducible, whereas the other promoter (for example, a second promoter) is inducible. As such, when the host cells transformed with such vector are cultured under conditions suitable for activating one promoter (for example, the first promoter), only one chain (e.g., the light chain) is expressed. Then, after a desirable period of expression of the first chain (e.g., the light chain), culturing conditions are changed to those suitable for the activation of the other promoter (for example, the second promoter), and hence inducing the expression of the second chain (e.g., the heavy chain). In one preferred embodiment, the light chain is expressed first followed by the heavy chain. In another embodiment, the heavy chain is expressed first followed by the light chain.

Specifically, according to one preferred embodiment, the recombinant vector comprises at least two translational units, one for the light-chain expression and the other for the heavy-chain expression. Moreover, the two translational units for light chain and heavy chain are under the control of different promoters. Promoters are untranslated sequences located upstream (5′) to the start of a coding sequence (generally within about 100 to 1000 bp) that control its expression. Such promoters typically fall into two classes, inducible and constitutive. Inducible promoters are promoters that initiate increased levels of transcription from DNA under their control in response to some change in culture conditions, e.g. the presence or absence of a nutrient or a change in temperature or pH.

For the purpose of this embodiment, either constitutive or inducible promoters can be used as the first promoter controlling the first-chain expression in time, and inducible promoters are used as the second promoter controlling the subsequent second-chain expression. In a preferred embodiment, both the first promoter and the second promoter are inducible promoters under tight regulation. A large number of promoters recognized by a variety of potential host cells are well known. The selected promoter sequence can be isolated from the source DNA via restriction enzyme digestion and inserted into the vector of the invention. Alternatively the selected promoter sequences can be synthesized. Both the native promoter sequence and many heterologous promoters may be used to direct amplification and/or expression of a target gene. However, heterologous promoters are preferred, as they generally permit greater transcription and higher yields of expressed target gene as compared to the native target polypeptide promoter.

Promoters suitable for use with prokaryotic hosts include the phoA promoter, the β-lactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to translational units encoding the target light and heavy chains using linkers or adaptors to supply any required restriction sites (Siebenlist et al., Cell, 20: 269 (1980)). Preferred promoters are phoA, tacI, tacII, Ipp, lac-Ipp, lac, ara, trp, trc and T7 promoters. More preferred promoters for use in this invention are the phoA promoter and the tacII promoter. Promoters that are functional in eukaryotic host cells are well known in the art, for example as described in U.S. Pat. No. 6,331,415. Examples of such promoters may include those derived from polyoma, Adenovirus 2, or Simian Virus 40 (SV40).

Each translational unit of the recombinant vector of the invention contains additional untranslated sequences necessary for sufficient expression of the inserted genes. Such essential sequences of recombinant vectors are known in the art and include, for example, the Shine-Dalgarno region located 5′- to the start codon and transcription terminator (e.g., λtₒ) located at the 3′-end of the translational unit.

Each translational unit of the recombinant vector further comprises a signal sequence component that directs secretion of the expressed chain polypeptides across a membrane. In general, the secretion signal sequence may be a component of the vector, or it may be a part of the target polypeptide DNA that is inserted into the vector. The secretion signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, lpp, or heat-stable enterotoxin II (STII) leaders, LamB, PhoE, PelB, OmpA and MBP. In a preferred embodiment of the invention, the signal sequences used in both translational units of the expression system are STII signal sequences or variants thereof. Preferably, the DNA encoding for such signal sequence is ligated in reading frame to the 5′-end of DNA encoding the light or heavy chain, resulting in a fusion polypeptide. Once secreted out of the cytoplasm of the host cell, the signal peptide sequence is enzymatically cleaved off from the mature polypeptide.

In another preferred aspect of the invention, in addition to the timing of the expression, the quantitative ratio of light- and heavy-chain expression is also modulated to maximize the yield of secreted and correctly assembled antibody. Such modulation is accomplished by simultaneously modulating translational strengths for light and heavy chains on the recombinant vector. One technique for modulating translational strength is disclosed in Simmons et al. U.S. Pat. No. 5,840,523. Briefly, the approach utilizes variants of the translational initiation region (TIR) within a translational unit. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes that can alter the amino acid sequence, although silent changes in the nucleotide sequence (as described below) are preferred. Alterations in the TIR can include, for example, alterations in the number or spacing of Shine-Dalgarno sequences, along with alterations in the signal sequence.

One preferred method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al., METHODS: A Companion to Methods in Enzymol., 4:151-158 (1992).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of antibodies also include invertebrate cells such as plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Examples of useful mammalian host cell lines are monkey kidney CV1 line transformed by SV40 (COS-7, ATCC CRL 1651); human embryonic kidney line (293 or 293 cells subcloned for growth in suspension culture (Graham et al., J. Gen Virol., 36:59 (1977)); baby hamster kidney cells (BHK, ATCC CCL 10); Chinese hamster ovary cells/-DHFR(CHO, Urlaub et al., Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

Prokaryotic cells used to produce the polypeptides of the invention are grown in media known in the art and suitable for culture of the selected host cells. Examples of suitable media include luria broth (LB) plus necessary nutrient supplements. In preferred embodiments, the media also contains a selection agent, chosen based on the construction of the expression vector, to selectively permit growth of prokaryotic cells containing the expression vector. For example, ampicillin is added to media for growth of cells expressing ampicillin-resistant gene. Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., and even more preferably is at about 30° C. The pH of the medium may be any pH ranging from about 5 to about 9, depending mainly on the host organism. For E. coli, the pH is preferably from about 6.8 to about 7.4, and more preferably about 7.0.

Eukaryotic host cells used to produce antibodies of the invention can be cultured in a variety of media known in the art. For example, commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing mammalian eukaryotic host cells. In addition, any of the media described in Ham and Wallace, Meth. Enz., 58: 44 (1979); Barnes and Sato, Anal. Biochem., 102:255 (1980); U.S. Pat. No. 4,767,704; 4,657,866; 4,927,762; or 4,560,655; WO 90/03430; WO 87/00195; U.S. Pat. Re. 30,985; or U.S. Pat. No. 5,122,469, may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Once the host cells are grown to a certain density, the culturing conditions are modified to promote the synthesis of the protein(s). If inducible promoter(s) are used in a dual-promoter vector as described above, protein expression is induced under conditions suitable for the activation of the promoter. In a preferred embodiment, both promoters are inducible. More preferably, the dual promoters are phoA and tacII, respectively. For example, a vector can be made wherein a phoA promoter is used for controlling transcription of the light chain, and a tacI promoter is used for controlling transcription of the heavy chain. During the first stage of induction, prokaryotic host cells transformed with such a phoA/tacII dual promoter vector are cultured in a phosphate-limiting medium for the induction of the phoA promoter and the expression of the light chain. After a desired period of time for light-chain expression, a sufficient amount of isopropyl-beta-D-thiogalactopyranoside (IPTG) is added to the culture for the induction of the tacII promoter and the production of the heavy chain.

In one aspect, if bacterial cells are employed as host cells, the antibody can be expressed in the cytoplasm. Various methods can be used to improve production of soluble and functional antibody in E. coli cytoplasm. For example, E. coli constrains deficient in the trxB gene have been found to enhance the formation of disulfide bonds in the cytoplasm and therefore useful for promoting expression of functional antibody molecules with proper disulfide bond formations in the cytoplasm. Proba et al., Gene, 159:203-207 (1995). Antibody variants can be made to replace cysteine residues such that the variant does not require formation of disulfide bonds in both V_{H} and V_{L}; such antibody variants, sometimes referred to as “intrabodies,” can therefore be made in a reducing environment that is not compatible with efficient disulfide bridge formation, such as in bacteria cytoplasm. Proba et al., J. Mol. Biol., 275:245-253 (1998).

When secretion signal sequences are used, the expressed light- and heavy-chain polypeptides are secreted into, and recovered from, the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant filtered and concentrated for further purification of the antibody produced. The expressed antibodies can be further isolated and identified using commonly known methods such as polyacrylamide gel electrophoresis (PAGE) and Western blot assay.

The antibody may be produced in large quantity by fermentation processes. Various large-scale fed-batch fermentation procedures are available for production of recombinant proteins. Large-scale fermentations have at least 1000 liters of capacity, preferably about 1,000 to 100,000 liters of capacity. These fermentors use agitator impellers or other suitable means to distribute oxygen and nutrients, especially glucose (the preferred carbon/energy source). Small-scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD₅₅₀ of about 180-270. A variety of inducers may be used, according to the vector construct employed, as is known in the art and described above. Cells may be grown for shorter periods prior to induction. Cells are usually induced for about 12-50 hours, although longer or shorter induction time may be used.

To further improve the production yield and quality of the antibody herein, various fermentation conditions can be modified. For example, to improve the proper assembly and folding of the secreted antibody, additional vectors overexpressing chaperone proteins, such as Dsb proteins (DsbA, DsbB, DsbC, DsbD and or DsbG) or FkpA (a peptidylprolyl cis,trans-isomerase with chaperone activity) can be used to co-transform the host prokaryotic cells. The chaperone proteins have been demonstrated to facilitate the proper folding and solubility of heterologous proteins produced in bacterial host cells. Chen et al., J. Bio. Chem., 274:19601-19605 (1999); U.S. Pat. Nos. 6,083,715 and 6,027,888; Bothmann and Pluckthun, J. Biol. Chem., 275:17100-17105 (2000); Ramm and Pluckthun, J. Biol. Chem., 275:17106-17113 (2000); Arie et al., Mol. Microbiol., 39:199-210 (2001).

To minimize proteolysis of expressed heterologous proteins (especially those that are proteolytically sensitive) such as in prokaryotic host cells, certain host strains deficient for proteolytic enzymes can be used for the present invention. For example, prokaryotic host cell strains may be modified to effect genetic mutation(s) in the genes encoding known bacterial proteases such as Protease III, OmpT, DegP, Tsp, TonA, PhoA, Protease I, Protease Mi, Protease V, Protease VI and combinations thereof. Some E. coli protease-deficient strains are available and described in, for example, Joly et al., Proc. Natl. Acad. Sci. USA, 95:2773-2777 (1998); U.S. Pat. Nos. 5,264,365 and 5,508,192; Hara et al. Microbial Drug Resistance, 2:63-72 (1996). Most preferably, it has the genotype containing Δptr or Δprc pre-suppressor.

In certain embodiments, an immunoconjugate comprising the antibody conjugated with a cytotoxic agent is made and used. Preferably, the immunoconjugate and/or antigen to which it is bound is/are internalized by the cell, resulting in increased therapeutic efficacy of the immunoconjugate in killing the target cell to which it binds. In a preferred embodiment, the cytotoxic agent targets or interferes with nucleic acid in the target cell.

Conjugates of an antibody and one or more small-molecule toxins, such as a calicheamicin, a maytansine (U.S. Pat. No. 5,208,020), a trichothene, and CC1065, are also contemplated herein.

In one preferred embodiment of the invention, the antibody is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody (Chari et al., Cancer Research, 52: 127-131 (1992)) to generate a maytansinoid-antibody immunoconjugate.

Another immunoconjugate of interest comprises an antibody conjugated to one or more calicheamicin molecules. The calicheamicin family of antibiotics is capable of producing double-stranded DNA breaks at sub-picomolar concentrations. Structural analogues of calicheamicin that may be used include, but are not limited to, γ₁^{I}, α₂^{I}, α₃^{I}, N-acetyl-γ₁^{I}, PSAG and θ^{I}₁ (Hinman et al., Cancer Research, 53: 3336-3342 (1993) and Lode et al., Cancer Research, 58: 2925-2928 (1998)). See also, U.S. Pat. Nos. 5,714,586; 5,712,374; 5,264,586; and 5,773,001.

Enzymatically active toxins and fragments thereof that can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g. a ribonuclease or a DNA endonuclease such as a deoxyribonuclease; DNase).

A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the antibody and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker may be a “cleavable linker” facilitating release of the cytotoxic drug in the cell. For example, an acid-labile linker, peptidase-sensitive linker, dimethyl linker or disulfide-containing linker (Chari et al., Cancer Research, 52: 127-131 (1992)) may be used.

Alternatively, a fusion protein comprising the antibody and cytotoxic agent may be made, e.g. by recombinant techniques or peptide synthesis.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionuclide).

The antibody may also be used in ADEPT by conjugating the antibody to a prodrug-activating enzyme that converts a prodrug (e.g., a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to convert it into its more active, cytotoxic form.

Enzymes that are useful in the ADEPT method include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature, 328:457-458 (1987)). Antibody-abzyme conjugates can be prepared as described herein for delivery of the abzyme to a tumor cell population.

The enzymes can be covalently bound to the antibodies herein by techniques well known in the art such as the use of the heterobifunctional crosslinking reagents discussed above. Alternatively, fusion proteins comprising at least the antigen-binding region of an antibody of the invention linked to at least a functionally active portion of an enzyme of the invention can be constructed using recombinant DNA techniques well known in the art (see, e.g., Neuberger et al., Nature, 312:604-608 (1984)).

The antibody herein can be used to increase tumor penetration. In this case, it may be desirable to modify the antibody in order to increase its serum half-life. This may be achieved, for example, by incorporation of a salvage receptor binding epitope into the antibody (e.g., by mutation of the appropriate region in the antibody or by incorporating the epitope into a peptide tag that is then fused to the antibody at either end or in the middle, e.g., by DNA or peptide synthesis). See WO 96/32478 published Oct. 17, 1996.

The salvage receptor binding epitope generally constitutes a region wherein any one or more amino acid residues from one or two loops of an Fc domain are transferred to an analogous position of the antibody. Even more preferably, three or more residues from one or two loops of the Fc domain are transferred. Still more preferred, the epitope is taken from the CH2 domain of the Fc region (e.g., of an IgG) and transferred to the CH1, CH3, or V_{H} region, or more than one such region, of the antibody. Alternatively, the epitope is taken from the CH2 domain of the Fc region and transferred to the C_{L} region or V_{L} region, or both, of the antibody.

Covalent modifications of the antibodies herein are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues.

Exemplary covalent modifications of polypeptides are described in U.S. Pat. No. 5,534,615. A preferred type of covalent modification of the antibody comprises linking the antibody to one of a variety of non-proteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

In another aspect, the present invention also concerns a method of determining if a mammalian, e.g. human, patient diagnosed with cancer is likely to benefit from treatment with a TGF-β antagonist. The method comprises the steps of
(a) testing the sensitivity of cancer cells obtained from the patient to the growth-inhibitory effect of TGF-beta;
(b) obtaining a gene expression profile of the cancer cells obtained from the patient and comparing it with a gene expression profile of cancer cells obtained from an animal model that are responsive to treatment with a TGF-beta antagonist; and
(c) identifying the patient as likely to benefit from treatment with a TGF-beta antagonist if the cancer cells obtained from the patient are not sensitive to the growth-inhibitory effect of TGF-beta and have a gene expression profile similar to the gene expression profile of the cancer cells obtained from said animal model that are responsive to said treatment.

For purposes herein, “similar” means that the expression profiles resemble or track each other in one or more ways, by showing patterns of expression that are within about 80% to 100% identical in quantity or other measurable expression parameter depending on the assay or technique used to measure the gene expression profile, as described further below in detail, more preferably within about 90 to 100%, and more preferably within about 95 to 100% identical. The gene expression profiles of the cancer cells from the patient and from the animal model are generally obtained by the same technique or assay to facilitate comparison thereof.

A variety of TGF-β antagonists and methods for their production are known in the art and many more are currently under development (see for example, Dennis et al., U.S. Pat. No. 5,821,227). The specific TGF-β antagonist employed is not a limiting feature; any effective TGF-β antagonist as defined herein may be useful in the methods and compositions of this invention, such as the examples in the definition provided herein.

One ideal TGF-β antagonist has a high affinity for TGF-βs, is stable in vivo and in vitro for long-term use, and is capable in some way of discriminating between “pathological” TGF-β that is involved in causing or exacerbating a disease process, and “physiological” TGF-β that is involved in the maintenance of normal homeostasis and cellular function in multiple organ systems. Although an understanding of the mechanism(s) is not necessary in order to use the present invention, it is contemplated that in one embodiment of the present invention, if TGF-β is required to maintain normal homeostasis, is activated locally at the site of production, and binds rapidly to nearby receptors without being released from the cell, while pathological processes are associated with more widespread activation of TGF-β, then a relatively bulky antagonist like the SR2F, discussed above, which has no cell-surface binding domains, may have poor access to the cell-associated “physiological TGF-β,” but be capable of effectively neutralizing the “pathological” TGF-β. However, it is not intended that the present invention be limited to any particular mechanism(s).

If the cancer is breast cancer, including primary and metastatic breast cancers, the foregoing prognostic method may additionally include the step of determining the Her2 status of the patient, where Her2⁺ patients typically, although not always, are likely not to respond, or to respond poorly, to treatment with a TGF-beta antagonist alone.

If the patient is likely to benefit from treatment with a TGF-β antagonist, the foregoing steps might be followed by the administration of an effective amount of a TGF-β antagonist alone or in combination with an effective amount of any chemotherapeutic and/or cytotoxic agent and/or other treatment modalities, including radiation therapy.

Methods of gene expression profiling are well known in the art and are typically based either on hybridization analysis of polynucleotides or sequencing of polynucleotides. The most commonly used methods known in the art for the quantification of mRNA expression in a sample include northern blotting and in situ hybridization (Parker and Barnes, Methods in Molecular Biology, 106:247-283 (1999)); RNAse protection assays (Hod, Biotechniques, 13:852-854 (1992)); and reverse transcription polymerase chain reaction (RT-PCR) (Weis et al., Trends in Genetics, 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). Any of these methods, or other methods known in the art, can be used to determine the gene expression profile of a tumor cell obtained from a patient, such as a human patient, and an animal serving as a model of a cancer responsive to a TGF-β antagonist, such as a mouse model. In the case of human patients, the source of tumor cells can be a fresh, frozen or fixed and paraffin-embedded tissue sample, from which mRNA can be extracted and subjected to gene expression analysis.

Alternatively, proteomics techniques can also be used to compare the expression profile of a human and reference (e.g. mouse) cancer cell. A proteomic profile is a representation of the expression pattern of a plurality of proteins in a biological sample, e.g. a cancer tissue. The expression profile can, for example, be represented as a mass spectrum, but other representations based on any physicochemical or biochemical properties of the proteins are also included. Thus the expression profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel. Proteomics techniques are well known in the art, and are described, for example, in the following textbooks: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research: Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

In a further aspect, patients who do not respond, or respond poorly, to treatment with a TGF-β antagonist might be treated with a combination therapy, including administration of a dose of a TGF-β antagonist that has no significant anti-tumor effect when administered alone, but is effective against the tumor when combined with an effective amount of one or more chemotherapeutic or cytotoxic agents and/or radiation therapy.

In yet another aspect, the invention concerns the treatment of bone destruction or bone loss associated with a tumor metastasis in a mammalian, e.g. human, patient by administration to the patient of an effective amount of a TGF-β antagonist. Such bone destruction or bone loss can result from a variety of reasons, including primary and secondary cancers that infiltrate the bones. Treatment includes reversal of bone destruction or bone loss, and stopping or slowing down the pathological process of bone destruction or loss.

In another aspect, the invention provides the treatment of a mammalian patient diagnosed with cancer comprising administering to the patient an effective amount of a combination of a TGF-beta antagonist and a chemotherapeutic or cytotoxic agent, and optionally also treated with an effective dose of radiation therapy. The response of the patient to the combination is monitored. The method is such that the effective amount of the combination is lower than the sum of the effective amounts of said TGF-beta antagonist and said chemotherapeutic or cytotoxic agent when administered individually, as single agents. This cancer is preferably breast, such as metastatic breast, or colorectal cancer. The chemotherapeutic agent is preferably a taxoid.

In yet another aspect, the invention supplies treatment of a mammalian patient diagnosed with cancer comprising administering to the patient an effective amount of a combination of a TGF-beta antagonist and radiation therapy, optionally also with an anti-angiogenic agent such as an antibody that specifically binds VEGF. The method is such that the effective amount of the combination is lower than the sum of the effective amounts of said TGF-beta antagonist and said radiation therapy when administered individually, as single agents. Preferably the cancer is breast cancer, such as metastatic breast cancer, or colorectal cancer.

In a still further aspect, the invention provides treatment of a mammalian patient diagnosed with cancer comprising administering to the patient an effective amount of a combination of a TGF-beta antagonist and an anti-angiogenic agent, optionally also with an effective amount of a chemotherapeutic or cytotoxic agent, and monitoring the response of the patient to the combination. This anti-angiogenic agent is preferably an antibody specifically binding VEGF. In one aspect, the method is such that the effective amount of the combination is lower than the sum of the effective amounts of said TGF-beta antagonist and said anti-angiogenic agent when administered individually, as single agents.

The TGF-beta antagonists herein can be used either alone or in combination with other compositions in a therapy. For instance, the antagonist may be co-administered with an antibody against other tumor-associated antigens than TGF-beta, such as one or more antibodies that bind to the EGFR, ErbB2, ErbB3, ErbB4, or VEGF antigens, chemotherapeutic agent(s) (including cocktails of chemotherapeutic agents), cytotoxic agent(s), anti-angiogenic agent(s), cytokines, and/or growth-inhibitory agent(s). It may be particularly desirable to combine the antibody with one or more other therapeutic agent(s) that also inhibit tumor growth. Alternatively, or additionally, the patient may receive combined radiation therapy (e.g. external beam irradiation or therapy with a radioactively labeled agent, such as an antibody). Such combined therapies noted above include combined administration (where the two or more agents are included in the same or separate formulations), and separate administration, in which case, administration of the antagonist can occur prior to, and/or following, administration of the adjunct therapy or therapies. Suitable dosages for the growth-inhibitory agent are those presently used and may be lowered when there is combined action (synergy) of the other agent(s) employed with the TGF-beta antagonist.

The TGF-beta antagonist (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antagonist is suitably administered by pulse infusion, particularly with declining doses of the antagonist. Preferably the dosing is given by injections, most preferably intravenous or subcutaneous injections, depending in part on whether the administration is brief or chronic.

The antagonist composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the antagonist, the type of antagonist, the method of administration, the scheduling of administration, and other factors known to medical practitioners. The antagonist need not be, but is optionally formulated with one or more agents currently used to prevent or treat the disorder in question. The effective amount of such other agents depends on the type and amount of antagonist present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of the antibody (when used alone or in combination with other agents such as chemotherapeutic, cytotoxic, growth-inhibitory, or anti-angiogenic agents, or antibodies to different antigens or cytokines as noted above) will depend on the type of disease to be treated, the type of antagonist, the severity and course of the disease, whether the antagonist is administered for preventive or therapeutic purposes, previous therapy, the patient's clinical history and response to the antagonist, and the discretion of the attending physician. The antagonist is suitably administered to the patient at one time or over a series of treatments. Depending on the type and severity of the disease, about 1 μg/kg to 15 mg/kg (e.g. 0.1 mg/kg-10 mg/kg) of antagonist, especially if it is an antibody, is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs.

The preferred dosage of the antagonist, especially antibody, will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses, may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Therapeutic formulations of the antagonist are prepared for storage by mixing the antagonist having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

As noted above, the formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Specifically, liposomes containing the antagonist may be prepared by such methods as described in Epstein et al., Proc. Natl. Acad. Sci. USA, 82:3688 (1985); Hwang et al., Proc. Natl. Acad. Sci. USA, 77:4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257:286-288 (1982) via a disulfide interchange reaction. A chemotherapeutic agent (such as doxorubicin) is optionally contained within the liposome. See Gabizon et al., J. National Cancer Inst., 81(19):1484 (1989).

The formulations to be used for in vivo administration must be sterile. This is readily accomplished by filtration through sterile filtration membranes.

Sustained-release preparations may be prepared. Suitable examples of sustained-release preparations include semi-permeable matrices of solid hydrophobic polymers containing the antagonist, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention includes use of cytotoxic chemotherapy in conjunction with treatment with soluble TGF-β antagonists. Embodiments include using cell-cycle active agents, (e.g., 5-fluorouracil) which show dose-limiting toxicity in tissue compartments with actively cycling cells, such as the bone marrow and gut. While an understanding of the mechanisms is not necessary in order to use the present invention, TGF-β keeps stem cells in a state of quiescence. The administration of a soluble TGF-β antagonist after a round of chemotherapy is contemplated to enhance stem cell proliferation and, thus, hematopoietic recovery (Sitnicka et al., Blood, 88:82-88 (1996)). Combination therapy with a soluble TGF-β antagonist and a chemotherapeutic agent leads to diminished toxicity of the chemotherapeutic agent in addition to the independently therapeutic effect of the TGF-β antagonist.

The present invention also includes treatment with soluble TGF-β antagonists in conjunction with immunotherapies. While an understanding of the mechanisms is not necessary in order to use the present invention, it is contemplated that secretion by tumors of inhibitors of the immune system limits the efficacy of immunotherapy approaches aimed at enhancing the immune recognition and destruction of the tumor (de Visser and Kast, Leukemia, 13:1188-1199 (1999)). TGF-β is an immunosuppressive agent that is highly secreted by tumors. Embodiments of the present invention include use of a TGF-β antagonist in combination with immunotherapy approaches (e.g., anti-tumor vaccination, adoptive immunotherapy) that result in a synergism between the anti-metastatic effects of the TGF-β antagonists and an enhanced efficacy of the immunotherapy.

Further details of the invention are provided in the following examples. The following examples are intended merely to illustrate the practice of the present invention and are not provided by way of limitation. The disclosures of all patent and scientific literatures cited herein are expressly incorporated in their entirety by reference.

### EXAMPLE 1

### Production and Characterization of Monoclonal Antibodies 2G7 and 4A11

A. Assay Procedures

I. ELISA Determination

96-well polystyrene assay plates were coated with 100 μl/well of purified TGF-beta1 at 1 μg/ml in pH 9.6 carbonate buffer for 18 hours at 4° C. Coated plates were blocked with 0.5% bovine serum albumin (BSA) in phosphate-buffered saline (PBS) (called BPBS) for one hour at 22° C., washed with 0.05% TWEEN2™ in PBS (called PBST), and incubated with 100 μl of hybridoma supernatants for one hour at 22° C. Plates were washed with PBST, and bound antibodies were detected with a goat anti-mouse IgG conjugated with peroxidase (Tago, Burlingame, Calif.). The plates were washed with PBST, and o-phenylenediamine dihydrochloride substrate was added at 100 μl/well. The reaction was stopped after 15 minutes and the optical density at 492 nm was determined on a UVMAX™ plate reader (Molecular Devices, Palo Alto, Calif.).

II. Iodination of rTGF-beta1

Purified TGF-beta1 was iodinated by a modified CHLORAMINE T™ (empirical formula: C₇H₇SO₂N NaCl (3H₂O)) procedure (Greenwood et al., Biochem. J., 89: 114 (1963)). Briefly, 10 μg of purified rTGF-beta1 was labeled with 1 mCi of Na¹25I on ice using three sequential additions of 20 μl of 0.1 mg/ml CHLORAMINE T™ separated by two-minute incubations. The reaction was stopped using sequential additions of 20 μl of 50 mM N-acetyl tyrosine, 1 M potassium iodine, followed by 200 μl of 8 M urea. The iodinated rTGF-beta1 was separated from free Na¹25I by HPLC using a C18 column and a trifluoroacetic acid/acetonitrile gradient, and fractions containing the main peak were pooled and stored at −70° C. (specific activity 112 μCi/μg).

III. Antigen Capture Radioimmunoassay

IMMUNLON™ 2 “REMOVAWELL”™ strips (Dynatech, Chantily, Va.) were coated with 5 μg/ml goat anti-mouse IgG (Boehringer Mannheim) in pH 9.6 carbonate for 18 hours at 4° C. The wells were washed with PBST, blocked with PBS containing 0.1% gelatin (called PBSG), washed with PBST, and incubated with hybridoma supernatants for four hours at 22° C. The wells were washed with PBST, and approximately 75,000 CPM/well of ¹25I-rTGF-beta1, in 100 μl of 0.1% gelatin in PBST, was added and incubated for two hours at 22° C. The plates were washed with PBST, and bound ¹25I-rTGF-beta1 was quantitated on a GAMMAMASTER™ counter (LKB, Sweden).

IV. Immunoprecipitation of ¹25I-rTGF-beta

The specificity of anti-TGF-beta monoclonal antibodies was also evaluated by their ability to immunoprecipitate ¹25I-rTGF-beta1 or porcine, platelet-derived ¹25I-TGF-beta2 (R & D Systems, Minneapolis, Minn.; specific activity 103.4 μCi/μg). Two μg of purified monoclonal antibody was incubated with 5×10⁴ CPM of ¹25I-rTGF-beta1 or ¹25I-TGF-beta2 for two hours at 22° C. The immunocomplexes were pelleted with protein A-SEPHAROSE™ bead-formed agarose-based gel filtration matrix (Repligen, Cambridge, Mass.) coated with rabbit anti-mouse IgG (Boehringer Mannheim Biochemicals, Indianapolis, Ind.) and subsequently washed 3× with PBST. The complexes were dissociated from the protein A-SEPHAROSE™ bead-formed agarose-based gel filtration matrix with reducing sample buffer and electrophoresed into 12% SDS-polyacrylamide gel (SDS-PAGE) and exposed to autoradiography.

V. Affinity Determination of TGF-beta Monoclonal Antibodies

The solid-phase radioimmunoassay procedure described by Mariani et al, J. Immunol. Methods, 71: 43 (1984) was used to determine the affinities of the TGF-beta-specific monoclonal antibodies. Briefly, purified anti-TGF-beta monoclonal antibodies were coated on IMMUNLON™ 2 “REMOVAWELL”™ strips in pH 9.6 carbonate buffer for 18 hours at 4° C. The wells were washed and blocked as described above. 40,000 CPM/well of either ¹25I-rTGF-beta1 or porcine ¹25I-TGF-beta2 (R & D Systems), in 50 μl PBSG, was added to 2-fold serial dilutions of non-labeled rTGF-beta1 or porcine TGF-beta2 ranging from 2500 to 9.7 ng/well, in 50 μl PBSG. The resulting mixture was incubated for 18 hours at 4° C. The wells were washed and counted as described above and the affinity constants determined by Scatchard analysis (Munson and Pollard, Anal. Biochem., 107: 220 (1980)), which yields similar results as the non-linear regression analysis of Antoni and Mariani, J. Immunol. Meth., 83: 61 (1985).

VI. Purification of Monoclonal Antibodies from Ascites Fluid

Parental hybridoma cultures secreting antibody that was positive in the above assays were cloned by limiting dilution and grown in ascites fluid in Balb/c mice (Potter et al., JNCI, 49: 305 (1972)) primed with PRISTANE™ primer. The monoclonal antibodies were purified from ascites fluid over protein A-SEPHAROSE™ bead-formed agarose-based gel filtration matrix and eluted in 0.1 M acetic acid, 0.5 M NaCl, pH 2.4 using established procedures (Goding, J. Immunol. Methods, 20: 241 (1978)) and stored sterile in PBS at 4° C.

VII. Monoclonal Antibody Neutralization of In Vitro TGF-beta Specific Activity

The in vitro TGF-beta assay used the mink lung fibroblast cell line, Mv-3D9 (subcloned from Mvl Lu, which is available from the American Type Culture Collection, Manassas, Va., as ATCC No. CCL-64). Briefly, purified anti-TGF-beta monoclonal antibodies and controls were incubated with either rTGF-beta1, native porcine TGF-beta2 (R & D Systems), or rTGF-beta3 (Derynck et al., Nature, 316: 701-705 (1985)) at a final concentration of 1000-2000 μg/ml for 18 hours at 4° C. Fifty μl of these mixtures were added to 96-well microtiter plates followed by 1×10⁴ Mv-3D9 cells, in 50 μl of minimal essential media containing 2 mM glutamine and 5% fetal bovine serum, and incubated for 18-24 hours at 37° C. in 5% CO₂. The wells were pulsed with 1 μCi of ³H-thymidine in 20 μl and harvested after four hours at 37° C. and counted in a scintillation counter. The percent inhibition of ³H-thymidine uptake for each dilution of TGF-beta standard was used to calculate the TGF-beta activity in pg/ml of the negative control monoclonal antibody and TGF-beta-specific, monoclonal antibody-treated samples.

VIII. Isotyping of Monoclonal Antibodies

Isotyping of TGF-beta1-reactive monoclonal antibodies was performed using the PANDEX™ fluorescence screen machine technology. Rat anti-mouse IgG antisera-coated polystyrene particles were used to bind the monoclonal antibody from culture supernatant dispensed into PANDEX™ 96-well assay plates. The plates were washed and FITC-conjugated rat monoclonal anti-mouse isotype specific reagents (Becton Dickinson Monoclonal Center) added. The bound fluorescence was quantitated by the PANDEX™ fluorescence screen machine technology.

IX. Epitope Analysis

Purified anti-rTGF-beta1 monoclonal antibodies were coupled to horseradish peroxidase (HRP) by the method of Nakane and Kawaoi, J. Histochem. Cvtochem., 22:1084 (1974). rTGF-beta1-coated plates were incubated with 50 μg/ml of purified anti-rTGF-beta1 or negative control in PBS for two hours at 22° C. A predetermined dilution of the anti-rTGF-beta monoclonal antibody-HRP conjugate was then added to the plates and incubated for one hour at 22° C. The plates were washed and substrate was added and reactivity quantitated as described above. The percent blocking of the heterologous anti-rTGF-beta1 monoclonal antibodies was compared to the autologous, positive blocking control.

X. Immunoblot Analysis

One μg/lane of rTGF-beta1 was electrophoresed in 12% SDS-PAGE using non-reducing sample buffer to determine the reactivities of the various monoclonal antibodies with the dimer forms of rTGF-beta1. The peptides were transblotted onto nitrocellulose paper and probed with the appropriate monoclonal antibody conjugated with HRP. Bound antibody was visualized using the insoluble substrate 4-chloro-1-naphthol (Kirkegaard and Perry, Gathersburg, Md.). The reaction was stopped after 15 minutes by exhaustive washing with distilled water and the immunoblots were dried and photographed.

B. Production of Anti-TGF-beta1- and Anti-TGF-beta2-Specific Monoclonal Antibodies

In the initial immunization protocols, Balb/c mice were immunized with rTGF-beta1 (produced and purified as described by Derynck et al., Nature, supra) by subcutaneous and intraperitoneal routes using a variety of immunogen preparations, doses, and schedules and using both complete and incomplete Freund's adjuvant. The immunization schedules were continued for up to 11 weeks. Several mice responded with measurable but low anti-rTGF-beta1 titers and two of these mice were sacrificed and their spleens used for fusions. From 1152 parental cultures only 84 positive anti-TGF-beta supernatants were detected. Ten of these hybridomas were cloned and resulted in monoclonal antibodies of low affinity that could not be used for assay development or purification.

As an alternative strategy, a group of ten Balb/c female mice (Charles River Breeding Laboratories, Wilmington, Mass.) were injected with 5 μg/dose of purified TGF-beta1 in 100-μl DETOX™ adjuvant (RIBI ImmunoChem Res. Inc., Hamilton, Mont.) in the hind footpads on days 0, 3, 7, 10, and 14. On day 17 the animals were sacrificed, their draining inguinal and popliteal lymph nodes were removed, and the lymphocytes were dissociated from the node stroma using stainless-steel mesh. The lymphocyte suspensions from all ten mice were pooled and fused with the mouse myeloma line X63-Ag8.653 (Kearney et al., J. Immunol., 123:1548 (1979)) using 50% polyethylene glycol 4000 by an established procedure (Oi and Herzenberg, in Selected Methods in Cellular Immunology, B. Mishel and S. Schiigi, eds., (W.J. Freeman Co., San Francisco, Calif., 1980), p. 351). The fused cells were plated into a total of 1344 96-well microtiter plates at a density of 2×10⁵ cells/well followed by HAT selection (Littlefield, J. W., Science, 145: 709 (1964)) on day 1 post fusion.

1190 of the wells were reactive with immobilized recombinant TGF-beta1 in the ELISA test. Eighteen of these cultures remained stable when expanded and cell lines were cryopreserved. These parental cultures were isotyped and assayed for their ability to capture ¹25I-rTGF-beta1 and to neutralize in vitro TGF-beta1 activity. From the 18 parental cultures that were assayed for neutralization of rTGF-beta1 and subsequently isotyped, two were of the IgG1 kappa isotype; the remainder were of the IgG2b kappa isotype. Only the monoclonal antibodies belonging to the IgG1 subclass were found to demonstrate rTGF-beta1 inhibitory (neutralization) activity in vitro. Three stable hybridomas were selected that secreted high-affinity anti-TGF-beta monoclonal antibodies. The characterization of these antibodies is detailed further below.

C. Immunoprecipitation of Radioiodinated TGF-beta

Immunoprecipitation experiments were performed to determine the ability of the three monoclonal antibodies to recognize and precipitate TGF-beta1 in solution. The autoradiograph showed that the anti-TGF-beta monoclonal antibodies 2G7, 4A11, and 12H5 immunoprecipitated equivalent amounts of ¹25I-rTGF-beta1, whereas the control monoclonal antibody 6G12 was negative. The immunoprecipitated bands had an apparent molecular weight of approximately 14.5 kD. A competitive inhibition assay was used to determine the affinity of interaction between TGF-beta1 and each of the monoclonal antibodies. Monoclonal antibodies 2G7 and 4A11 had equally higher affinities, which were 1.2×10⁸ l/mole.

Immunoprecipitation experiments were also performed to determine the ability of the monoclonal antibodies selected to recognize and precipitate TGF-beta2 in solution. The autoradiograph showed that, in contrast to rTGF-beta1, only antibody 2G7 immunoprecipitated ¹25I-TGF-beta2 to any measurable degree. Comparison of 4A11 and 12H5 to the negative control reveals little specific precipitation. These results were surprising in that cross-blocking experiments revealed that 4A11 and 2G7 were able to inhibit the binding of one another to human rTGF-beta1. See Table 1.

TABLE 1

| | Percent Crossblocking of Mabs to TGF-beta1 | | | |
|---|---|---|---|---|
| Binding Monoclonal | Blocking Monoclonal Antibody | | | |
| Antibody | 2G7 | 4A11 | 12H5 | 456* |
| 2G7 | 100 | 74 | 32 | 1.9 |
| 4A11 | 96 | 100 | 19 | 1.5 |
| 12H5 | 28 | 12 | 100 | 3.4 |

*Mab 456 is a control antibody that reacts with CD4.

Taken together, the data indicate that the epitopes recognized by these two monoclonal antibodies are distinct, but are either in close proximity or somehow affect the binding of one another from a distance. From both the immunoprecipitation and cross-blocking experiments, 12H5 appears to be a distinct epitope, although some blocking was observed. This conclusion is also supported by the neutralization data below.

D. Immunoblot Analysis with rTGF-beta1

Since the active form of TGF-beta is a homodimer, immunoblots were performed to determine whether the monoclonal antibodies recognized this form. The antibodies 2G7, 4A11 and 12H5 all reacted in an indirect immunoblot with the TGF-beta1 dimer (nonreduced) form. 2G7 gave a much stronger band than either 4A11 or 12H5. As in the immunoprecipitation experiment, control antibody 6G12 was negative. This pattern of reactivity was also observed in a direct Western blot with HRP conjugates of these monoclonal antibodies.

In summary, the protocol employing footpad immunizations coupled with fusions of the draining lymph nodes was performed after multiple unsuccessful attempts at breaking tolerance to rTGF-beta1 using a variety of immunization procedures and dosing schedules in Balb/c and C3H mice with complete and incomplete Freund's adjuvant. In general, this procedure was found useful to generate a rapid response with very high affinity to these weak immunogens, in contrast to the experience of Dasch et al., J. Immunol., 142: 1536-1541 (1989), who generated a TGF-beta1- and TGF-beta2-neutralizing monoclonal antibody using purified bovine bone-derived TGF-beta2 in Freund's adjuvant as immunogen in Balb/c mice.

All three monoclonal antibodies bound to rTGF-beta1 in the immunoblot, ELISA, cross-blocking, and immunoprecipitation assays. Two of the anti-rTGF-beta antibodies neutralized rTGF-beta1 activity in vitro, while only one of the two neutralized both TGF-beta2 and TGF-beta3 activity in the mink lung fibroblast cell assay. The TGF-beta1-neutralizing antibodies also blocked radioiodinated rTGF-beta1 binding in a radioreceptor assay, indicating that the in vitro neutralization of rTGF-beta1 activity may be due to receptor blocking.

### EXAMPLE 2

### Humanized 2G7 Antibodies

The variable domains of murine monoclonal antibody 2G7 were first cloned into a vector that allows production of a mouse/human chimeric Fab fragment. Total RNA was isolated from the hybridoma cells using a STRATAGENE™ RNA extraction kit following manufacturer's protocols. The variable domains were amplified by RT-PCR, gel purified, and inserted into a derivative of a pUC119-based plasmid containing a human kappa constant domain and human CH1 domain as previously described (Carter et al,. Proc. Natl. Acad. Sci. (USA), 89: 4285 (1992) and U.S. Pat. No. 5,821,337). The resultant plasmid was transformed into E. coli strain 16C9 for expression of the Fab fragment. Growth of cultures, induction of protein expression, and purification of Fab fragment were as previously described (Werther et al,. J. Immunol., 157: 4986-4995 (1996); Presta et al., Cancer Research, 57: 4593-4599 (1997)).

DNA sequencing of the chimeric clone allowed identification of the CDR residues (Kabat et al., supra). Using oligonucleotide site-directed mutagenesis, all six of these CDR regions were introduced into a complete human framework (V_{L} kappa subgroup I and V_{H} subgroup III) contained on plasmid VX4 as previously described (Presta et al., Cancer Research, 57: 4593-4599 (1997)). Protein from the resultant “CDR-swap” was expressed and purified as above. Binding studies were performed to compare the two versions. Briefly, a NUNC MAXISORP™ plate was coated with 1 microgram per ml of TGF-beta extracellular domain (ECD; produced as described in WO 90/14357) in 50 mM of carbonate buffer, pH 9.6, overnight at 4° C., and then blocked with ELISA diluent (0.5% BSA, 0.05% POLYSORBATE™ 20, PBS) at room temperature for 1 hour. Serial dilutions of samples in ELISA diluent were incubated on the plates for 2 hours. After washing, bound Fab fragment was detected with biotinylated murine anti-human kappa antibody (ICN 634771) followed by streptavidin-conjugated horseradish peroxidase (Sigma) and using 3,3′,5,5′-tetramethyl benzidine (Kirkegaard & Perry Laboratories, Gaithersburg, Md.) as substrate. Absorbance was read at 450 nm. Binding of the CDR-swap Fab was significantly reduced compared to binding of the chimeric Fab fragment.

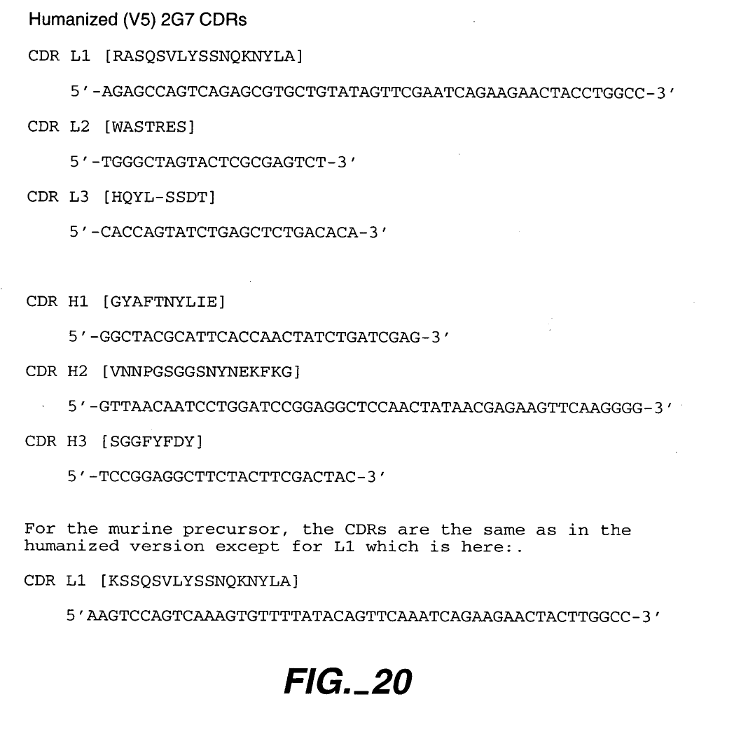

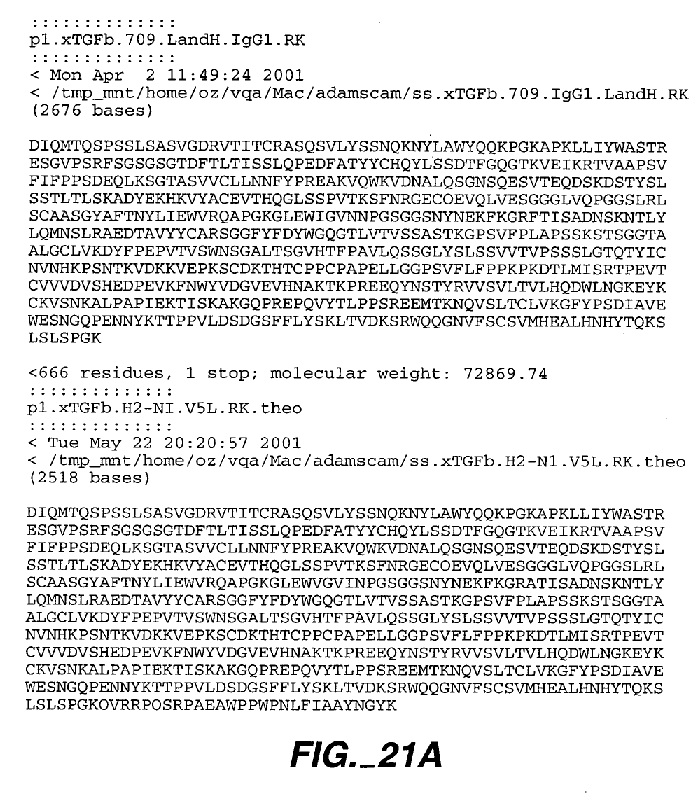

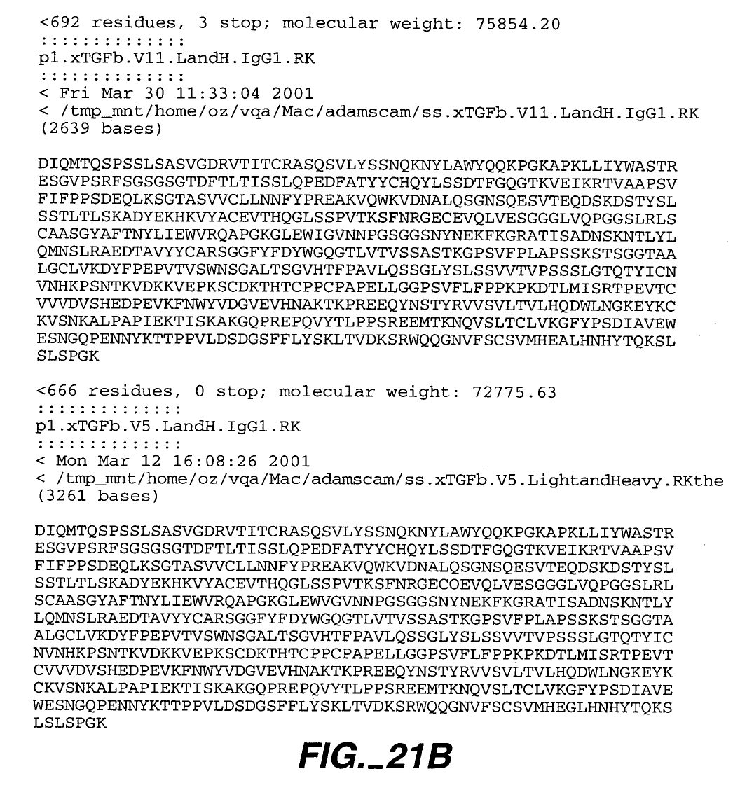

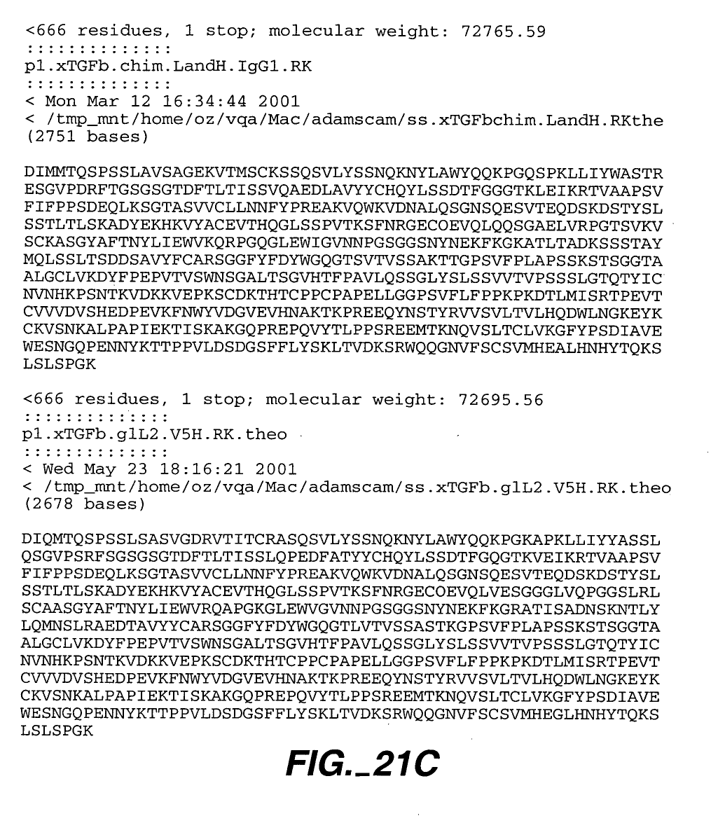

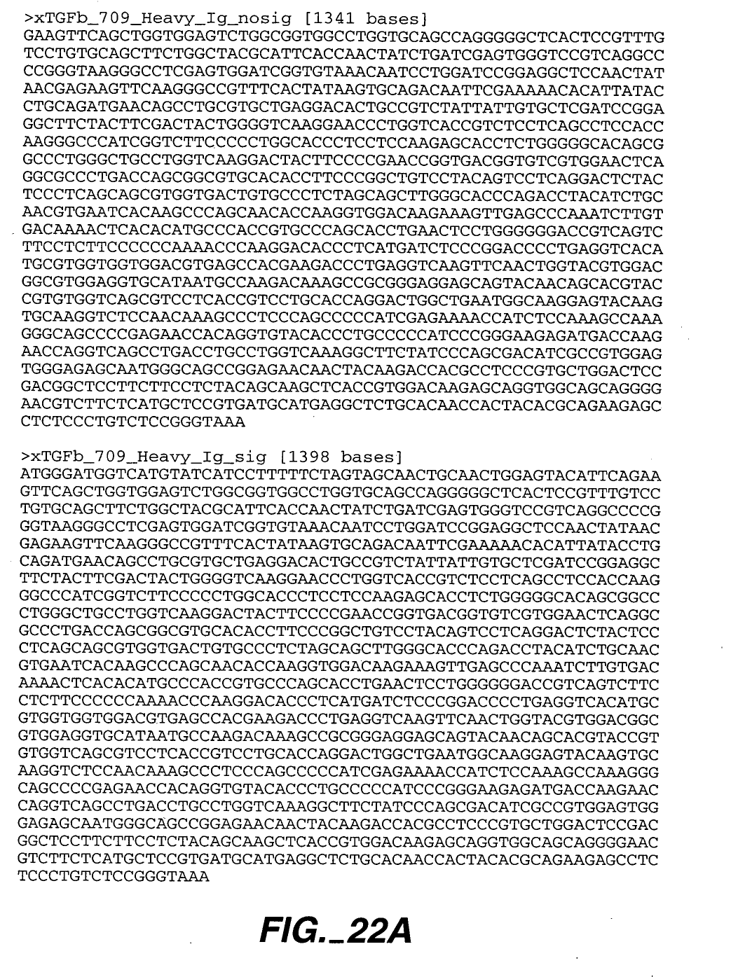

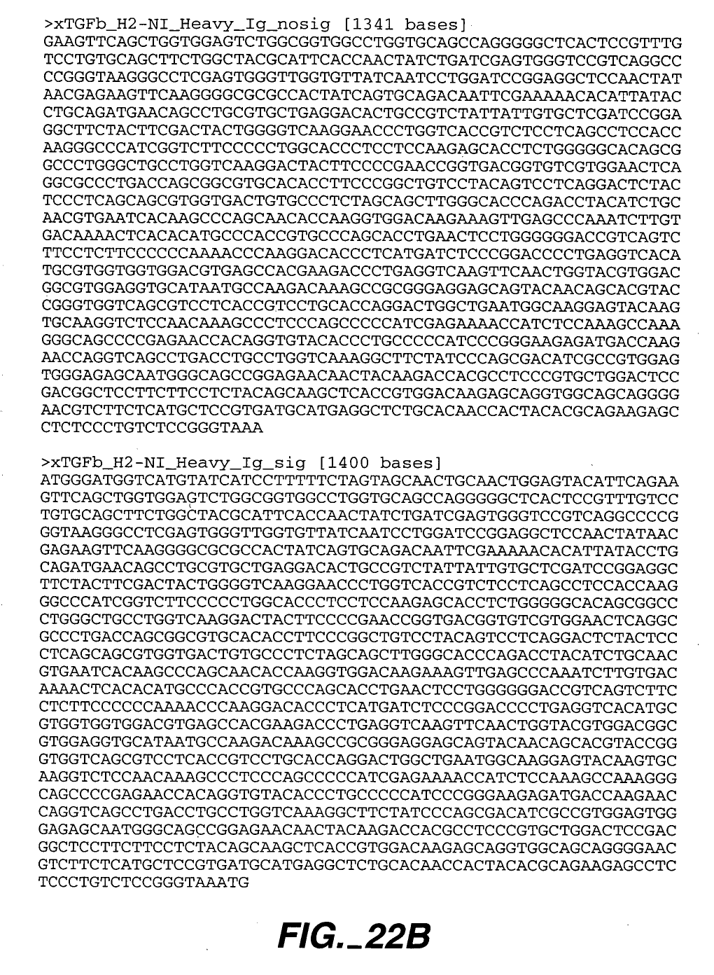

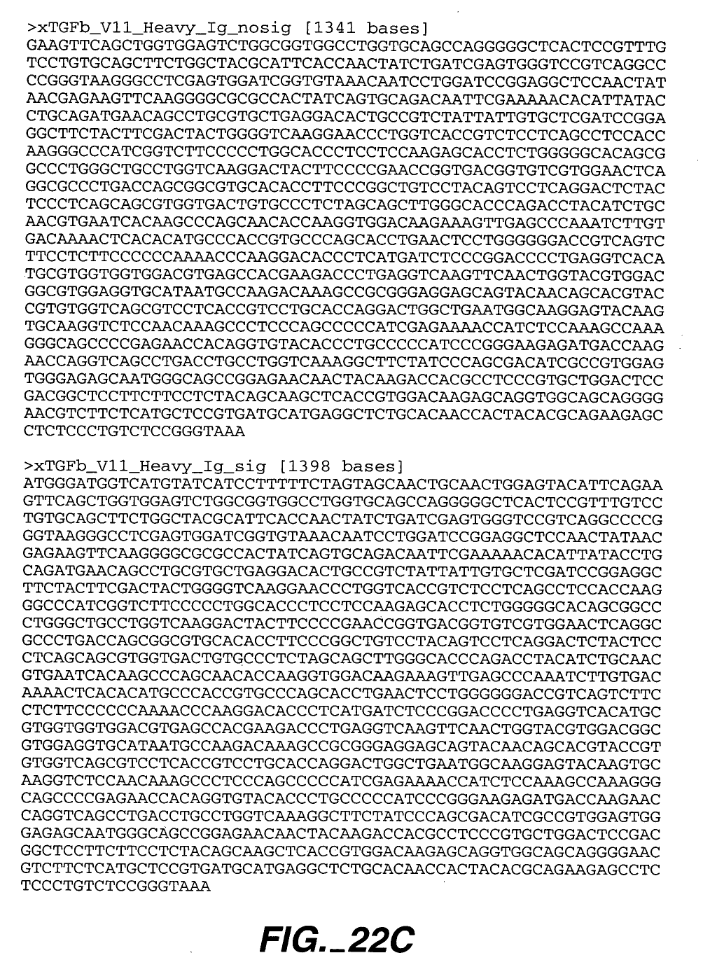

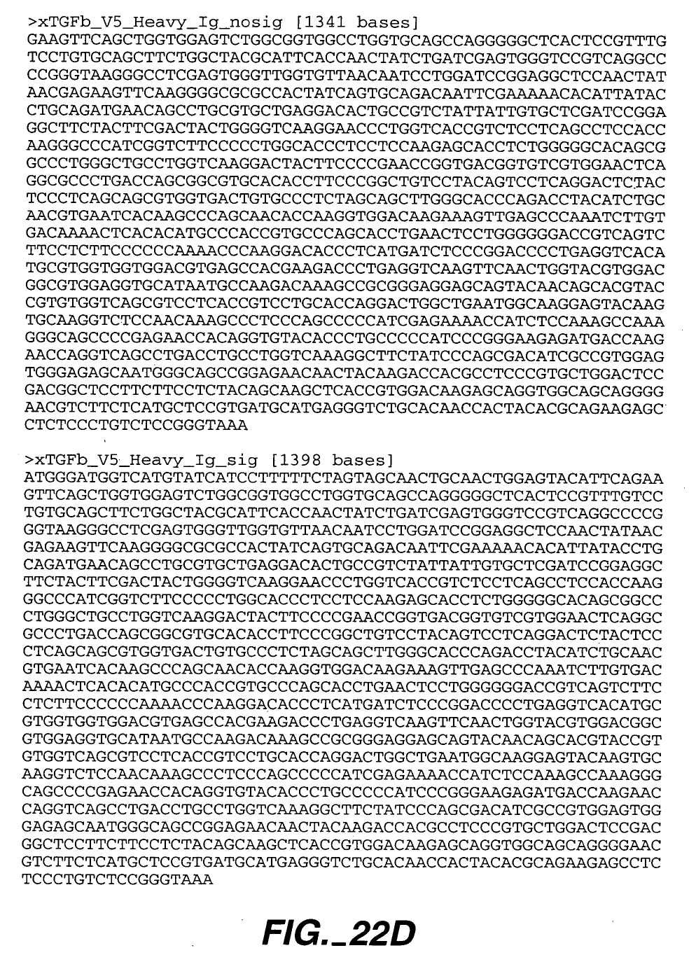

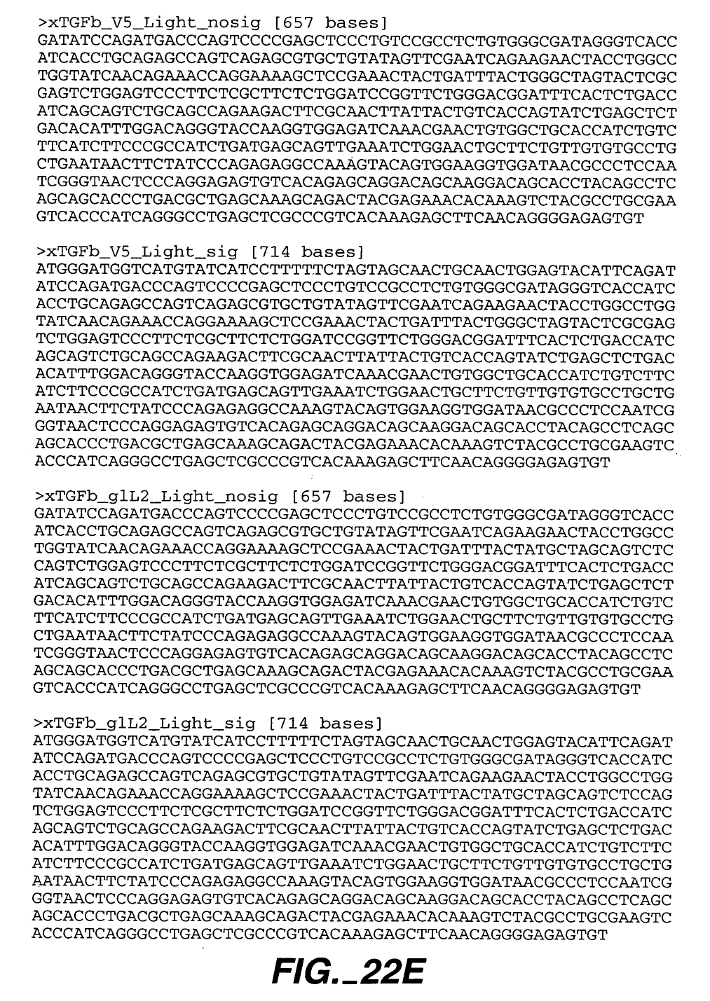

To restore binding of the humanized Fab, mutants were constructed using DNA from the CDR-swap as template. Using a computer-generated model, these mutations were designed to change human framework region residues to their murine counterparts at positions where the change might affect CDR conformations or the antibody-antigen interface. Mutants are shown in Table 2. (Note that all amino acid numbering is expressed as in Kabat et al., supra.) For sequences, see FIGS. 19-22.

TABLE 2
Designation of Humanized 2G7 FR Mutations

| | Framework region (FR) substitutions as |
|---|---|
| | compared to human anti-TGF-beta consensus |
| Mutant no. | sequence (SEQ ID NO:6) |
| Version 3 | ArgH71Ala |
| Version 4 | ArgH71Ala, AlaH49Gly, |
| Version 5 | ArgH71Ala, AlaH49Gly, PheH67Ala |
| Version 6 | ArgH71Ala, AlaH49Gly, LeuH78Ala |
| Version 709 | ArgH71Ala, AlaH49Gly, ValH48Ile |
| Version 710 | ArgH71Ala, AlaH49Gly, IleH69Leu |
| Version 11 | ArgH71Ala, AlaH49Gly, AsnH73Lys |
| Version 712 | ArgH71Ala, AlaH49Gly, IleH69Leu, AsnH73Lys |

Versions 3 and 4 were used as intermediates to obtain the humanized Fab versions bearing later numbers. Version 5, with the changes AlaH49GIy, PheH67Ala, and ArgH71 Ala, appears to have binding restored to that of the original chimeric 2G7 Fab fragment, as do Versions 709 and 11. Versions 710 and 712 are expected to have similar binding to the chimeric fragment, but version 712 has an additional framework mutation that might not be desirable due to the possibility of increased immunogenicity. Additional FR or CDR residues, such as L3, L24, L54, and/or H35, may be modified (e.g. substituted as follows: GlnL3Met, ArgL24Lys, ArgL54Leu, GluH35Ser). Substitutions that might be desirable to enhance stability are the substitution of leucine or isoleucine for methionine to decrease oxidation, or the change of asparagines in the CDRs to other residues to decrease the possibility of de-amidation. Alternatively, or additionally, the humanized antibody may be affinity matured (see above) to further improve or refine its affinity and/or other biological activities.

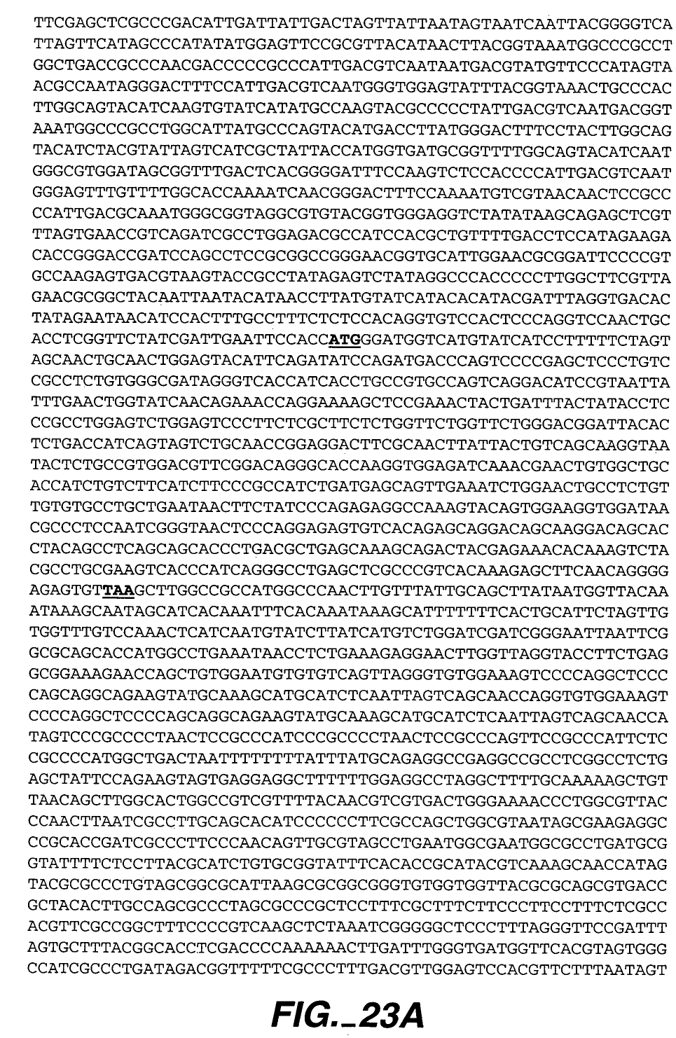

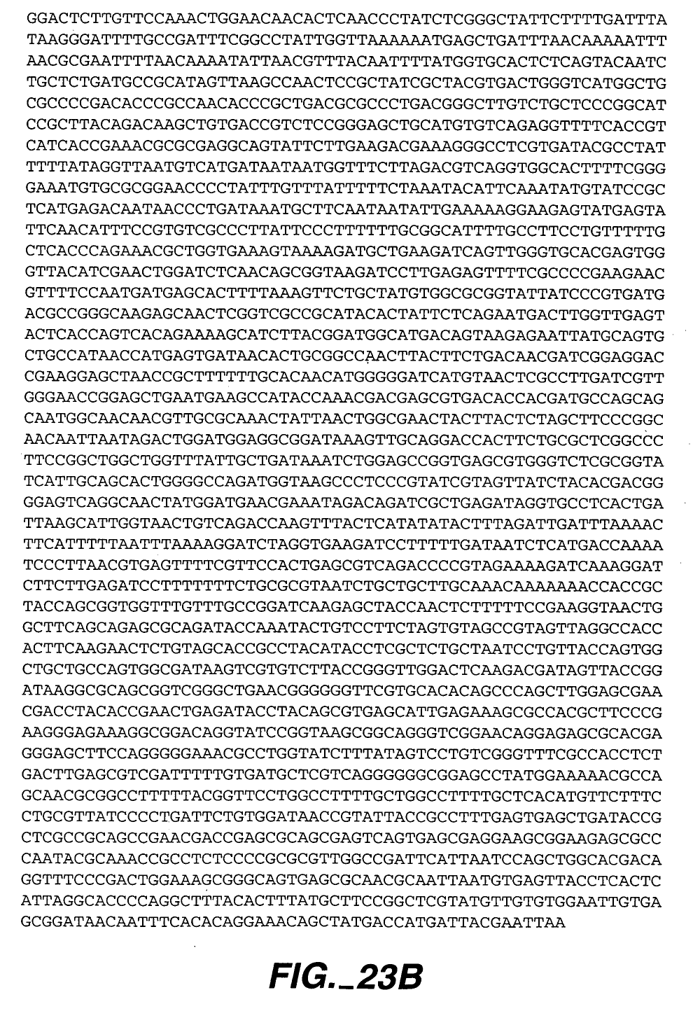

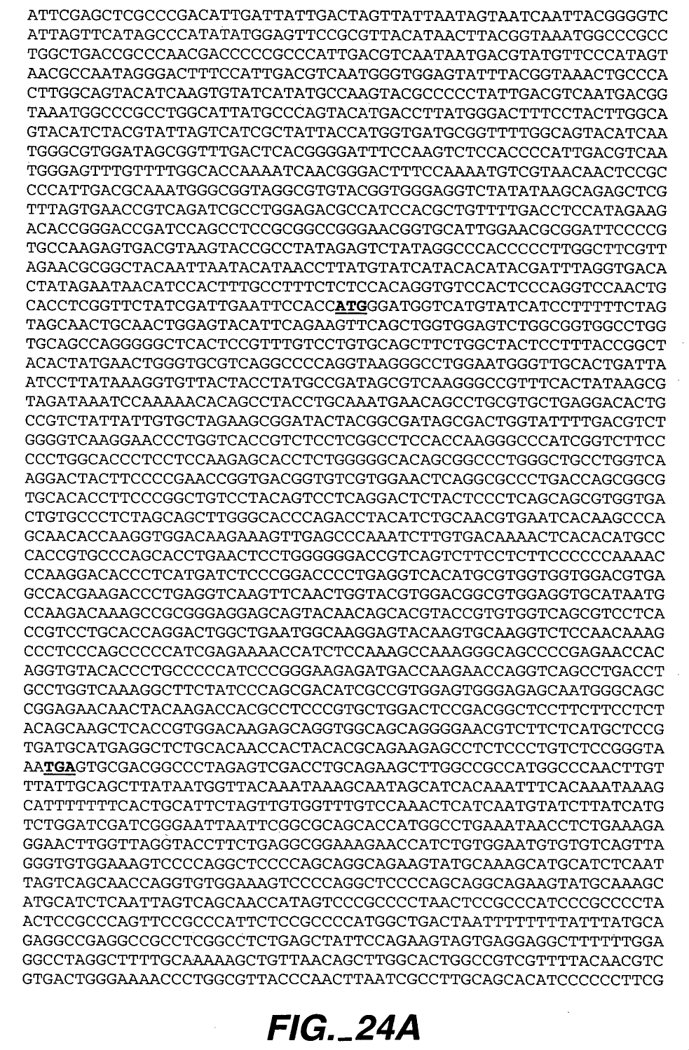

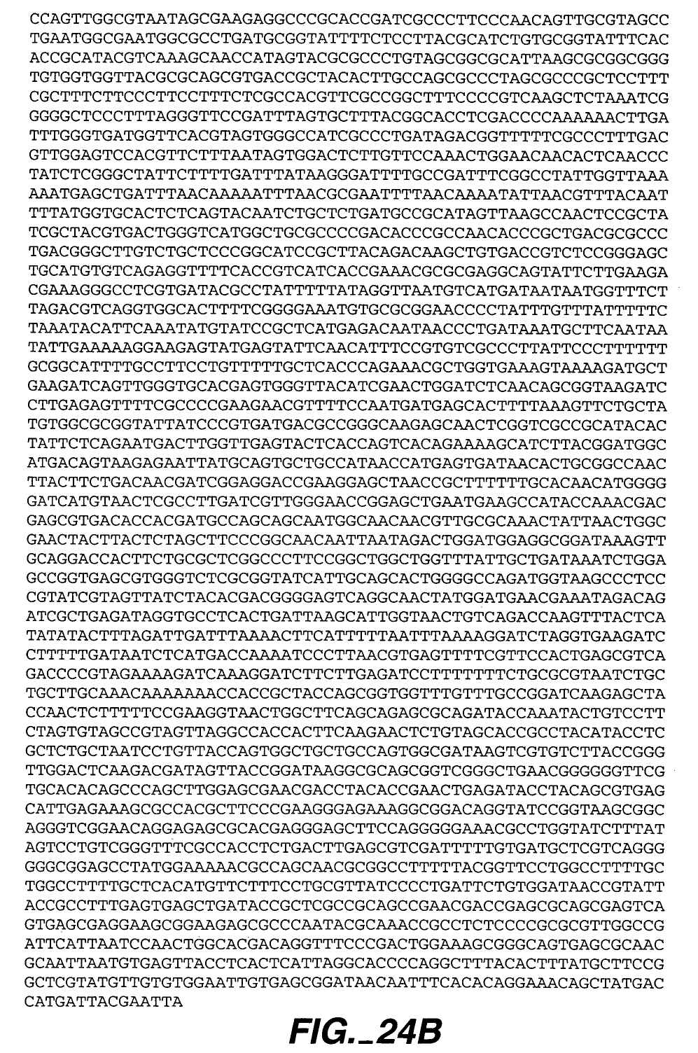

Plasmids for expression of full-length IgG's were constructed by subcloning the VL and VH domains of chimeric 2G7 Fab as well as humanized Fab versions 5, 709, and 11 into previously described pRK vectors for mammalian cell expression (Gorman et al., DNA Prot. Eng. Tech., 2:3-10 (1990)). Briefly, each Fab construct was digested with EcoRV and Blpl to excise a VL fragment, which was cloned into the EcoRV/Blpl sites of plasmid pDR1 (see FIG. 23) for expression of the complete light chain (VL-CL domains). Additionally, each Fab construct was digested with PvuII and ApaI to excise a VH fragment, which was cloned into the PvuII/ApaI sites of plasmid pDR2 (see FIG. 24) for expression of the complete heavy chain (VH-CH1-CH2-CH3 domains).

For each IgG variant, transient transfections were performed by co-transfecting a light-chain expressing plasmid and a heavy-chain expressing plasmid into an adenovirus-transformed human embryonic kidney cell line, 293 (Graham et al., J. Gen. Virol., 36:59-74, (1977)). Briefly, 293 cells were split on the day prior to transfection, and plated in serum-containing medium. On the following day, a calcium phosphate precipitate was prepared from double-stranded DNA of the light and heavy chains, along with PADVANTAGE™DNA (Promega, Madison, Wis.), and added drop-wise to the plates. Cells were incubated overnight at 37° C., then washed with PBS and cultured in serum-free medium for 4 days at which time conditioned medium was harvested. Antibodies were purified from culture supernatants using protein A-SEPHAROSE CL-4B™ bead-formed agarose-based gel filtration matrix, then buffer exchanged into 10 mM sodium succinate, 140 mM NaCl, pH 6.0, and concentrated using a CENTRICON-10® centrifugal filter device (Amicon). Protein concentrations were determined by measuring absorbance at 280 nm or by quantitative amino acid analysis.

Additional modifications to hu2G7 Version 5 IgG were made in order to clarify which CDRs contributed to binding, which CDRs could be reverted to the sequence of human germline kappa loci without loss of activity, or for stabilization of the antibody. These are named as shown in Table 3 as “Heavy chain.Light chain”, and the amino acid differences between version 5 and these versions are given.

TABLE 3
Designation of Humanized 2G7 CDR Mutations

| | CDR substitutions as compared to human anti- |
|---|---|
| Mutant no. | TGF-beta version 5. |
| Version 5 | |
| (V5H.V5L) | |
| H2N1.V5L | Same as Version 5 except Asn51 is changed to Ile |
| | in the CDR H2 |
| V5H.g1L2 | Same as Version 5 except the CDR L2 is reverted |
| | to the sequence of human germline kappa locus |
| | L8/L9/L14/L15: YASSLQS (SEQ ID NO:39) |
| V5H.g1L1glL2 | Same as Version 5 except the CDR L1 is reverted |
| | to the sequence of human germline kappa locus |
| | L8/L9: RASQGISSYLA (SEQ ID NO:37) and CDR |
| | L2 is reverted to the sequence of human germline |
| | kappa locus L8/L9/L14/L15: YASSLQS (SEQ ID |
| | NO:39) |
| H2Nl.g1L1glL2 | Same as Version 5 except the CDR L1 is reverted |
| | to the sequence of human germline kappa locus |
| | L8/L9: RASQGISSYLA (SEQ ID NO:37) and CDR |
| | L2 is reverted to the sequence of human germline |
| | kappa locus L8/L9/L14/L15: YASSLQS (SEQ ID |
| | NO:39), and Asn51 is changed to Ile in CDR H2. |

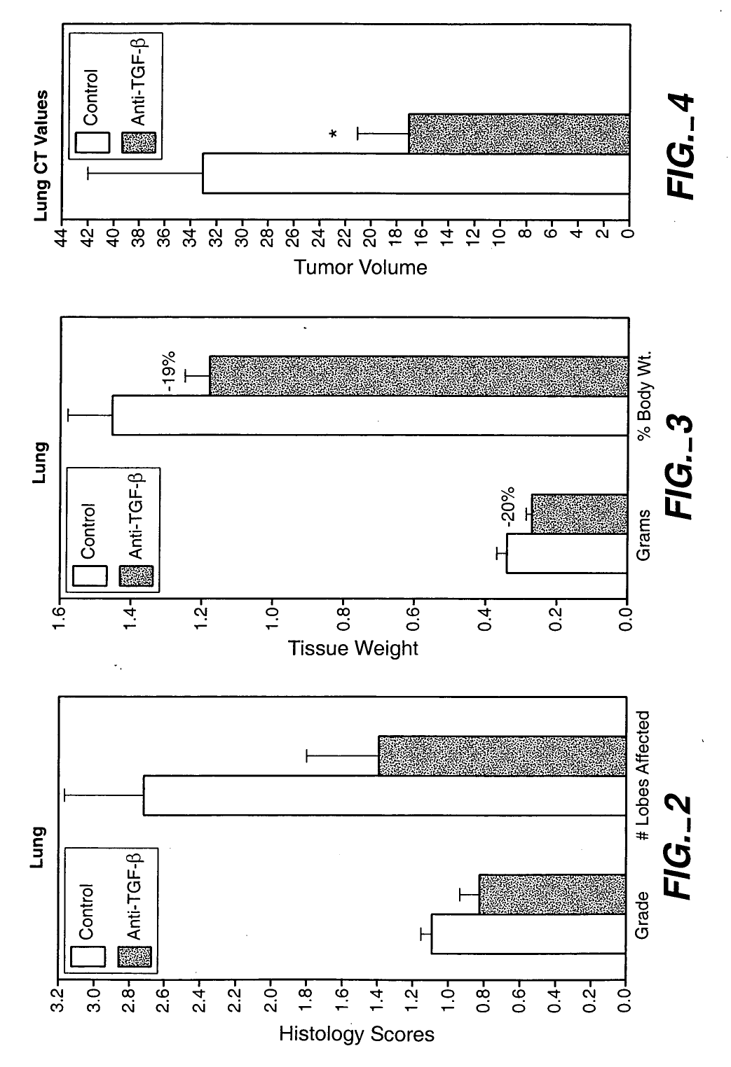

The name for the germline sequence used for CDR L1 is L8/L9, as set forth in FIG. 4 of Cox et al., Eur. J. Immunol., 24: 827-836 (1994) and in FIG. 2e of Schable and Zachau, Biol. Chem. Hoppe-Sevler, 374:1001-1022 (1993). For CDR12, the germlne sequence is named L8/L9 μl 4/L15 (see Cox et al, supra, and Schable and Zachau, supra).

Reversions to the sequence of human germline (gl) kappa locus were made in all the CDR's, but only the germline revertants set forth above showed binding. V5H.g1L2, with CDR L2 reverted to the sequence of the human germlne kappa locus, still bound to TGF-beta as well as V5H.V5L. The two versions V5H.g1L1 gIL2 and H₂NI.g1L1 gIL, as well as H₂NI.V5L, did not bind as well as the chimera.

A mouse messangial cell proliferation assay was used to test a control antibody and several humanized antibodies (V5H.V5L, V5H.gIL2, H₂NI.V5L, V5H.gIL1gIL2, and H2N1.gIL gIL2). The protocol is as follows:

On day 1: Mouse messangial cells were plated on a 96-well plate in Media (a 3:1 mixture of Dulbecco's modified Eagle's medium and Ham's F12 medium-95%-fetal bovine serum-5%-supplemented with 14 mM HEPES buffer) and grown overnight.

On day 2: TGF-beta with three different concentrations (100 ng, 10 ng and 1 ng) and five different types of humanized TGF antibody (20 μg/ml) were diluted in serum-free Media and added to the cells. A mouse TGF antibody was used as a control (2G7).

On day 4: After 48 hours incubation, 20 μl of reaction buffer (CELLTITER 96® AQUEOUS ONE SOLUTION REAGENT™ containing a tetrazolium compound 3-(4,5-dimethylthiazol-2-yl)-5-(3-carboxymethoxyphenyl)-2-(4-sulfophenyl)-2H-tetrazolium, inner salt, and an electron coupling reagent (phenazine ethosulfate) (Promega Inc. Cat number G3580)) was added to each well of the plate and allowed to incubate for 2 hours. The absorbance (OD) was measured at 490 nm.

H₂NI.V5L (20 μg/ml) completely blocked cell inhibition induced by TGF-beta at 1 ng/ml level, which is the same result as using the chimeric mouse control. Version 5 (V5H.V5L) also blocked cell inhibition similarly to the control.

Various humanized antibodies were tested for their activity in neutralizing various TGF-betas versus 2G7 using the 3T3 cell line from fibroblasts of disaggregated Swiss mouse embryos stimulated with one of three TGF-betas in vitro and then their proliferation was measured as activity. The humanized antibody H₂NI.V5L was quite superior in activity to the control 2G7 antibody. The other humanized antibodies tested, H₂NI.gIL2 (CDR L2 reverted to the sequence of the human germlne kappa locus) and V5H.gIL2 (CDR L2 reverted to the sequence of the human germlne kappa locus), showed comparable activity, with V5H.gIL2 being the least effective for all of TGF-beta1 through -beta3. Versions 709, 710, and 711 are the most preferred humanized versions, since they bind TGF-beta comparably as the chimeric antibody (chimH.chimL; 2G7 Fab fragment) and/or neutralize TGF-beta or block cell inhibition induced by TGF-betas in vitro, and have the fewest framework changes of all the humanized antibodies tested, which would minimize the risk of an immune response in patients. In addition, H2NI.V5L is a particularly preferred antibody, as it is clearly superior in neutralization activity and might have improved stability due to the changes in the CDR H2.

### EXAMPLE 3

### Study of Tumor Metastasis in Mouse Models of Metastatic Breast Cancer

A. 4T1 Model

In a first set of experiments, 4T1 cells were derived from a single spontaneously arising mammary tumor from a BALB/cfC₃H mouse. Primary 4T1 tumor cells were injected into mammary fat pads of immunocompetent BALB/c mice One week after injection, palpable primary tumors were observed. The tumor spontaneously metastasized into the lung (about two week after injection), liver and spleen (about three weeks after injection) and bone (between about 4 and 5 weeks after injection).

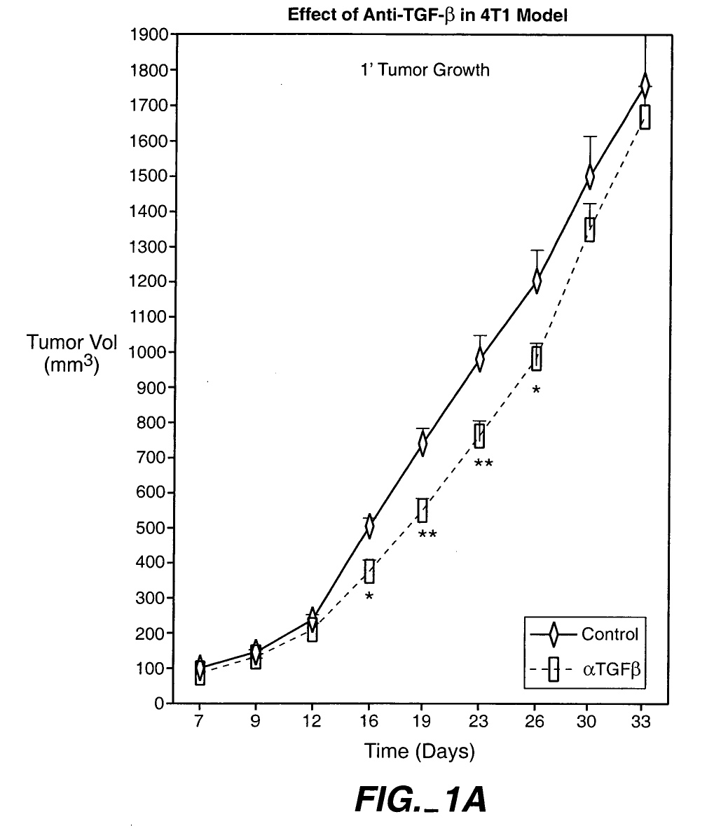

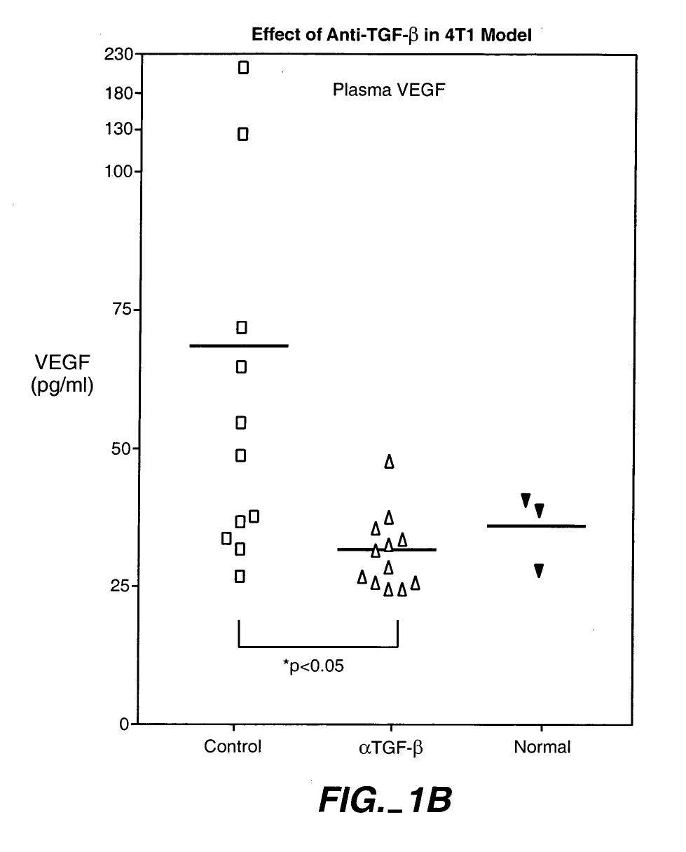

The animals were treated with 15 mg/kg, 25 mg/kg and 43 mg/kg doses of an anti-TGF-β antibody (2G7). Tests were carried out at day 0, 1, 2, and 1 and 2 weeks after injection of cancer cells. As shown in FIG. 1, treatment with a 43-mg/kg dose transiently decreased the size of primary tumor, and reduced systemic levels of VEGF. The 25-mg/kg dose was found to provide better results than the 15-mg/kg dose, while there was no significant difference between the results obtained with 25-mg/kg and 43-mg/kg doses, respectively (data for 15 mg/kg and 25 mg/kg doses not shown).

As shown in FIGS. 2, 3 and 4, early treatment with the anti-TGF-β antibody (5 weeks after injection of cells) decreased the histology scores (grade and number of lobes affected), weight and volume of secondary lung tumors. Histology scores were determined using the following scale, where “%” is the percentage of the tissue comprised of tumor cells, and “invasion” is an indication whether or not the tumor cells were noted in the blood vessels and/or lymph nodes:
Normal: Infiltration is minimal; % 1-33; no invasion.
Grade II: Moderate infiltration; % 34-66; some invasion.
Grade III: Severe infiltration; % 67-100; many invasions.

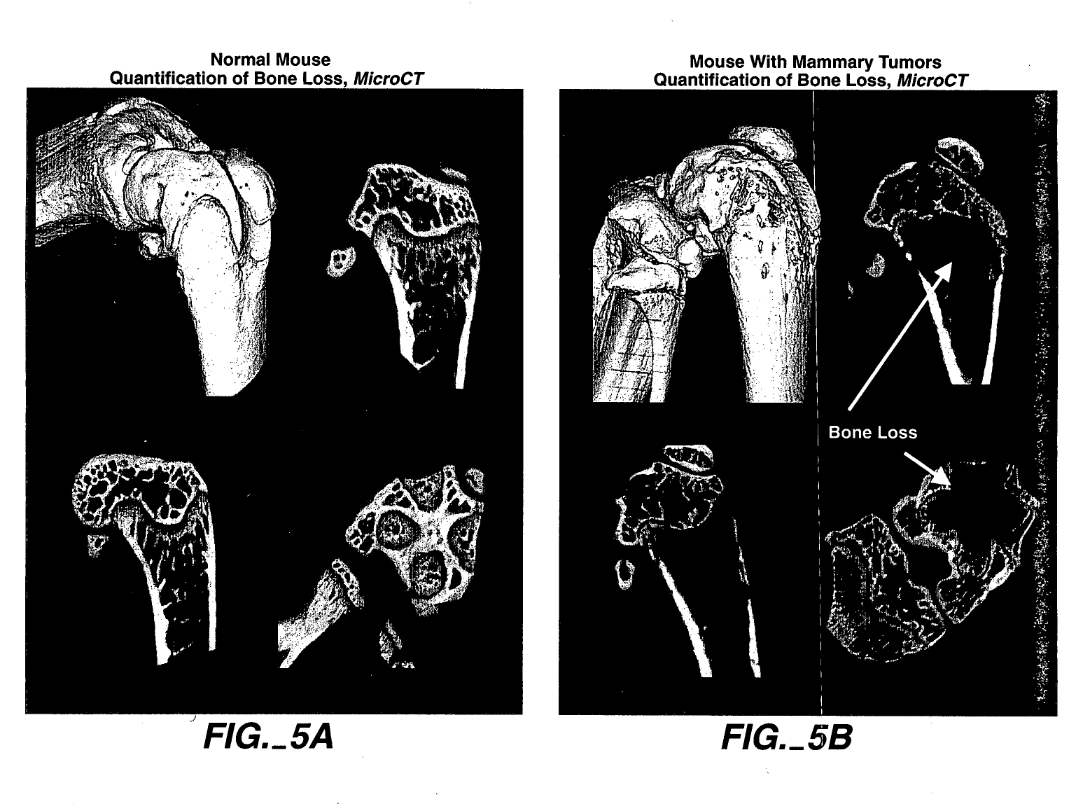

FIG. 5 shows the bone destruction by comparing MicroCT images of normal trabecular bone and bone metastasis.

Results of quantitative analysis of bone destruction are shown in the following Table 4.

TABLE 4

| | Trabecular | Trabecular | | | Mineral |
|---|---|---|---|---|---|
| | number | thickness | BV/TV | BS/BV | density |
| anti-TGF-β | −2.8%* | ns | −4.8% | ns | ns |
| antibody | | | | | |
| +cells | −7.2% | −22.5% | −28.2% | +28.6% | −15.9% |
| anti-TGF-β | +6.5%** | +7.2% | +14.3% | −6.6% | +6.3% |
| antibody + cells | | | | | |

*relative to control mice without tumors
** relative to mice with tumors treated with control antibodies
+cells = mice injected with tumor cells
anti-TGF-β antibody + cells = cells injected with tumor cells and treated with anti-TGF-β antibody.
BV = bone volume
TV = total volume
BS = bone surface

The results presented in Table 4 show that early treatment (5 weeks after injection of tumor cells) with an anti-TGF-β antibody (2G7) inhibited certain parameters of breast tumor-induced bone destruction.

B. Cells from Her2+Mammary Tumor

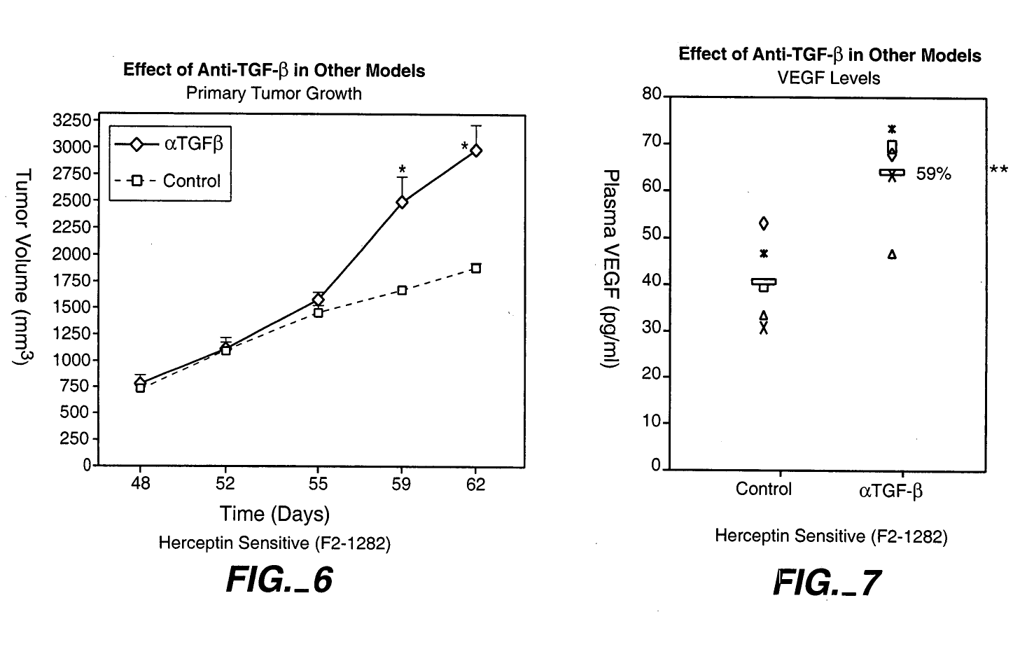

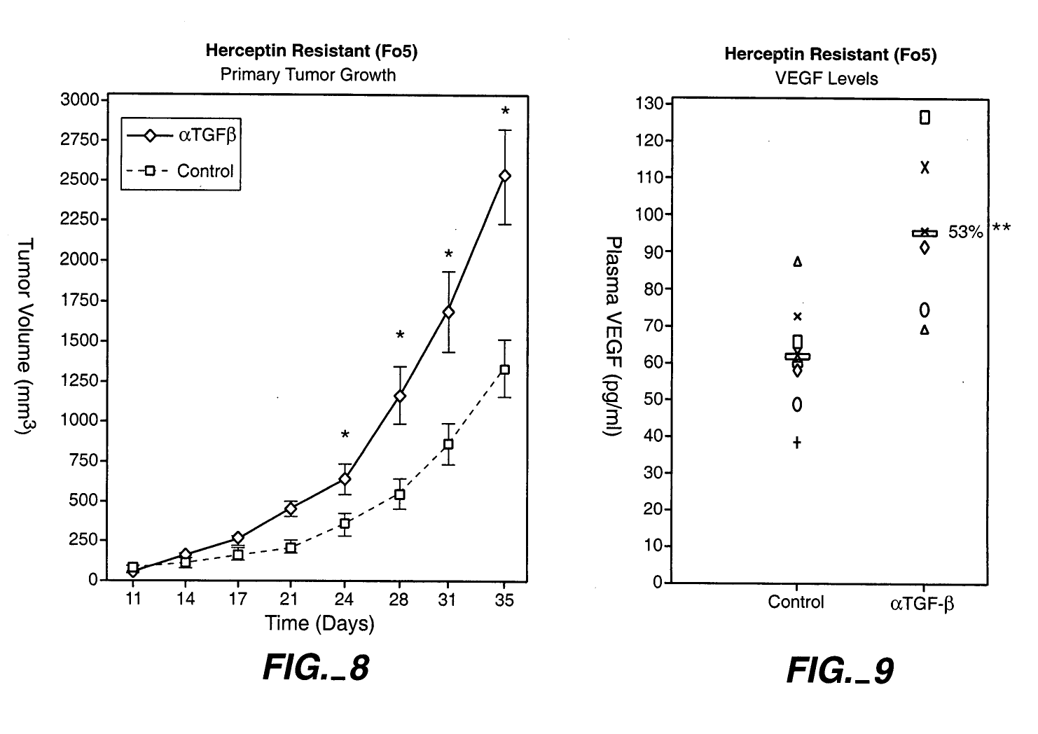

Epithelial cells from trastuzumab-sensitive (F2-1282) and trastuzumab-resistant (Fo5) tumor cell lines were injected into mammary fat pads of immunocompetent mice. As shown in FIGS. 6 and 7, and 8 and 9, respectively, treatment with a 25 mg/kg dose of an anti-TGF-β antibody (2G7) at day 0 (day of injecting tumor cells) increased the size of primary tumor, and systemic VEGF levels independent of the trastuzumab-responsiveness of the tumor.

Unlike 4T1 epithelial cells, Her2⁺ epithelial cells do not synthesize high levels of TGF-β, are growth inhibited by TGF-β, and grow slowly both in vitro and in vivo. Metastasis from such cells produces non-surface lung tumors (images not shown), the incidence and growth of which are not inhibited by anti-TGF-β antibody treatment.

C. PymT Tumors

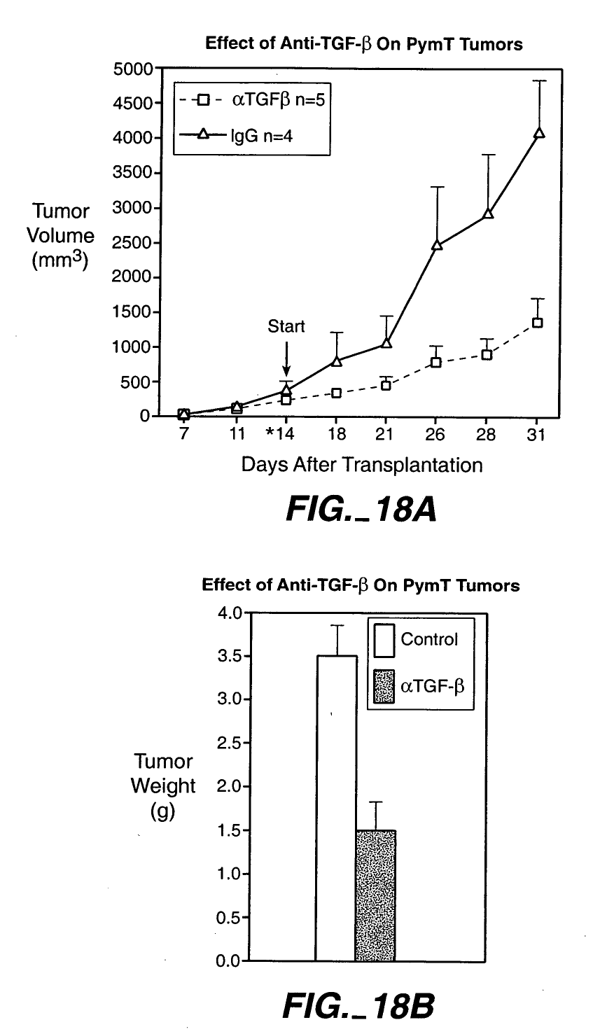

This is a mouse model of breast cancer caused by expression of the polyoma middle T oncoprotein (PyMT) in the mammary epithelium. Primary tumor cells from PyMT tumors were injected (2 million or 5 million cells) into the mammary fat pad of a recipient mouse. The tumor developed was then passaged in a large number of further mice. The data shown in FIG. 18 demonstrate that treatment with an anti-TGF-β antibody (2G7) decreased primary tumor growth.

### EXAMPLE 4

### Study of Tumor Metastasis in a Mouse Model of Metastatic Melanoma

This study used B16-F10 and B16-BL6 metastatic melanoma sublines, subcutaneously injected into immunocompetent mice, to test the effect of anti-TGF-β antibodies (2G7) on primary and secondary melanoma. In particular, C57Black 6 mice were injected subcutaneously with 500,000 tumor cells. The primary tumor developed was removed at day 14, and the mice were sacrificed around day 28. The anti-TGF-β antibody concentration was approximately 30 mg/kg. The B16-F10 subline is known to be able to colonize bone if introduced into the bone by direct injection (not as a result of subcutaneous injection). The B16-BL6 subline is metastatic to the lung.

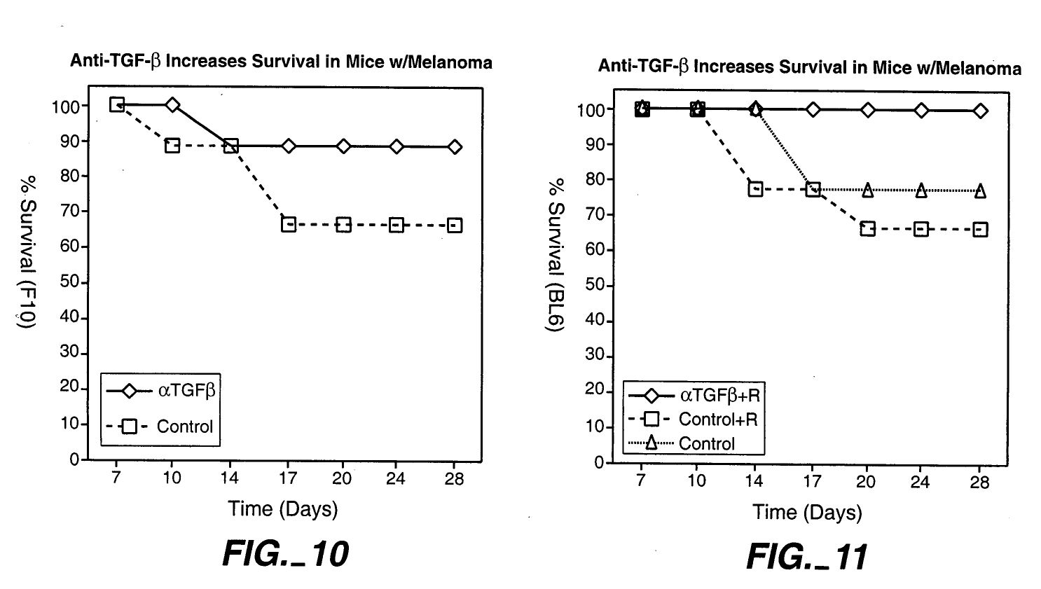

As shown in FIGS. 10 (F10) and 11 (BL6), treatment with an anti-TGF-β antibody 2G7 (about 30 mg/kg) increased survival of mice with melanoma.

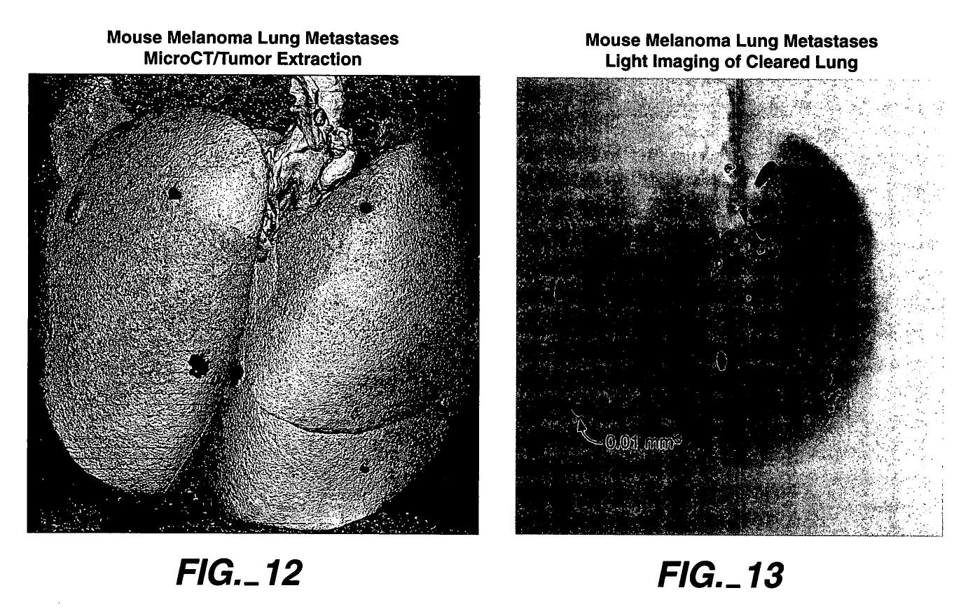

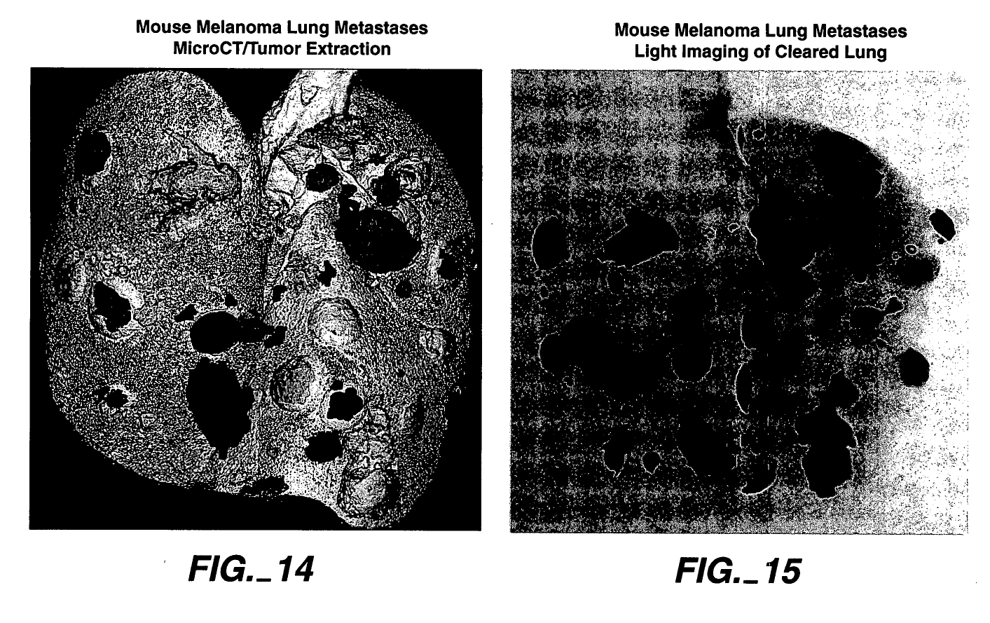

FIGS. 12-15 are various representations of melanoma lung metastases, including CT analyses and light imaging.

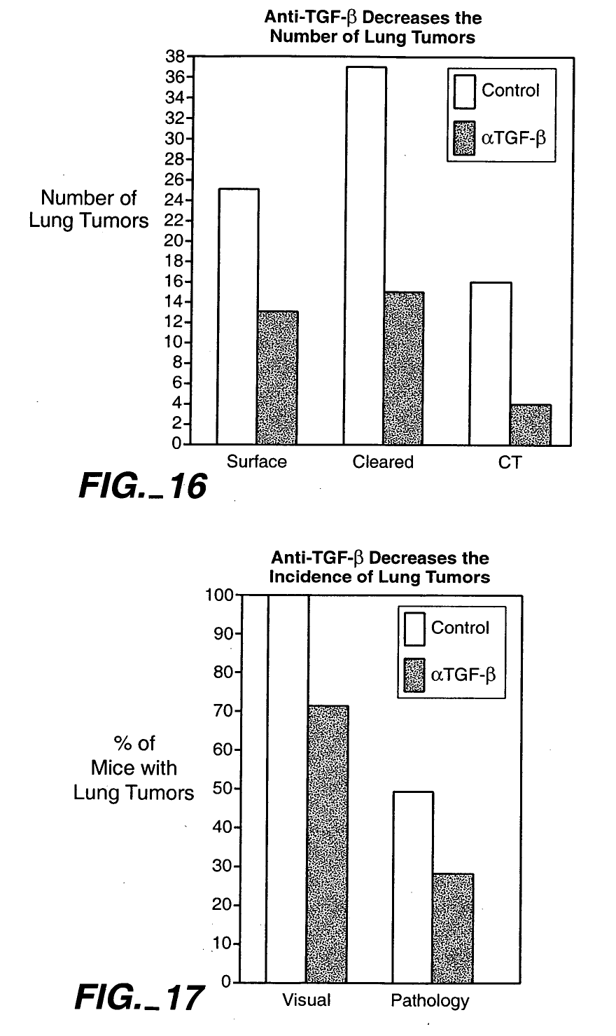

As shown in FIGS. 16 and 17, treatment with an anti-TGF-β antibody 2G7 significantly decreased both the number and the incidence of metastatic lung tumor in this model.

It is noted that, while not used in this experiment, further sublines of B16 are available, and can be used in similar experiments. Such further sublines include, for example, F0 (not metastatic), F1 (low metastasis; about 30%); and G3.26 (highly metastatic).

The animal models described in Examples 3 and 4 find further utility in screening assays to identify new molecules which might be involved in or might inhibit growth of primary and/or secondary tumors, for example by enhancing angiogenesis and/or stromal recruitment, although the utility of these assays is not limited by the action mechanism of the molecules identified.

Although the foregoing refers to particular embodiments, it will be understood that the present invention is not so limited. It will occur to those ordinary skilled in the art that various modifications may be made to the disclosed embodiments without diverting from the overall concept of the invention. All such modifications are intended to be within the scope of the present invention.

What is claimed is:

1. A method of screening comprising the steps of: (1) administering a plurality of test substances to a non-human syngeneic immunocompetent animal model bearing at least one soft tissue or bone metastasis, in the presence or absence of a primary tumor; (2) determining the effects of said test substances on the soft tissue or bone metastasis and growth of the primary tumor, if present; and (3) identifying a test substance that inhibits the growth of a soft tissue or bone metastasis, without adverse effect on the status of the primary tumor, if present.

2. The method of claim 1 wherein the soft tissue metastasis is present in a tissue selected from the group consisting of lung and liver tissue.

3. The method of claim 1 wherein the bone metastasis results in bone destruction.

4. The method of claim 1 wherein the animal model bears both soft tissue and bone metastases.

5. The method of claim 1 wherein the animal model bears a primary tumor.

6. The method of claim 1 wherein the primary tumor has been surgically removed from the animal.

7. The method of claim 1 wherein the animal is a rodent.

8. The method of claim 1 wherein the animal is a mouse or a rat.

9. The method of claim 1 wherein the animal is a mouse.

10. The method of claim 1 wherein the primary tumor is breast tumor.

11. The method of claim 10 wherein the breast tumor has developed from cells derived from a spontaneous mouse mammary carcinoma.

12. The method of claim 11 wherein the cells are 4T1 mouse mammary carcinoma cells.

13. The method of claim 10 wherein the primary breast tumor is Her2⁺, which has developed from epithelial cells produced from Her2⁺ tumors or from passages of Her2⁺ tumors.

14. The method of claim 13 wherein the primary breast tumor is trastuzumab-resistant.

15. The method of claim 13 wherein the primary breast tumor is trastuzumab-respondent.

16. The method of claim 10 wherein the primary breast tumor is a PymT tumor, which has developed from epithelial cells produced from PymT tumors or from passages of PymT tumors.

17. The method of claim 1 wherein the primary tumor is melanoma.

18. The method of claim 17 wherein the melanoma is of a B16 subline.

19. The method of claim 1 wherein the test substance identified is an antagonist of a secreted molecule.

20. The method of claim 1 wherein the test substance identified is a transforming growth factor-beta (TGF-beta) antagonist.

21. The method of claim 20 wherein the TGF-beta antagonist is an antibody specifically binding TGF-beta.

22. The method of claim 20 wherein the TGF-beta antagonist inhibits bone metastasis.

23. The method of claim 20 wherein the TGF-beta antagonist reduces bone destruction or bone loss.

24. The method of claim 1 wherein the test substances administered to said animal include a known chemotherapeutic or cytotoxic agent.

25. The method of claim 24 wherein the chemotherapeutic or cytotoxic agent is a taxoid.

26. The method of claim 24 wherein the animal is administered two test substances, one of which is a TGF-beta antagonist, and the other one the chemotherapeutic or cytotoxic agent, and the combined effects of the two test substances on soft tissue or bone metastasis and primary tumor growth, if primary tumor is present, are determined.

27. The method of claim 26 wherein the TGF-beta antagonist is an antibody specifically binding TGF-beta and the chemotherapeutic or cytotoxic agent is a taxoid.

28. The method of claim 1 wherein the animal is additionally exposed to an effective dose of radiation therapy.

29. A method of determining if a mammalian patient diagnosed with cancer is likely to benefit from treatment with a TGF-beta antagonist, comprising:
(a) testing the sensitivity of cancer cells obtained from the patient to the growth-inhibitory effect of TGF-beta;
(b) obtaining a gene expression profile of the cancer cells obtained from the patient and comparing it with a gene expression profile of cancer cells obtained from an animal model that are responsive to treatment with a TGF-beta antagonist; and
(c) identifying the patient as likely to benefit from treatment with a TGF-beta antagonist if the cancer cells obtained from the patient are not sensitive to the growth-inhibitory effect of TGF-beta and have a gene expression profile similar to the gene expression profile of the cancer cells obtained from said animal model that are responsive to said treatment.

30. The method of claim 29 wherein said cancer is breast cancer.

31. The method of claim 29 wherein the cancer is metastatic breast cancer.

32. The method of claim 30 further comprising the step of determining the Her2 status of breast cancer cells obtained from the patient, and identifying the patient as likely to respond to treatment with a TGF-β antagonist if the cells are Her2 negative.

33. The method of claim 29 wherein the patient is human.

34. The method of claim 29 wherein the TGF-beta antagonist is an antibody specifically binding TGF-beta.

35. The method of claim 29 wherein the patient has soft tissue metastasis.

36. The method of claim 35 wherein the soft tissue metastasis includes at least one of lung and liver metastases.

37. The method of claim 35 wherein the patient additionally has bone metastasis.

38. The method of claim 29 wherein the patient shows bone destruction or bone loss.

39. The method of claim 29 further comprising the step of administering to the patient an effective amount of a TGF-beta antagonist to treat the cancer.

40. The method of claim 39 wherein the treatment is performed after surgical removal of the primary breast cancer.

41. The method of claim 39 further comprising administering to the patient an effective amount of a chemotherapeutic or cytotoxic agent or an effective dose of radiation therapy to treat the cancer.

42. The method of claim 41 wherein the chemotherapeutic agent is a taxoid.

43. The method of claim 39 further comprising administering to the patient an effective amount of an antibody specifically binding Her2 to treat the cancer.

44. The method of claim 43 wherein said antibody is trastuzumab.

45. The method of claim 39 further comprising administering to the patient an effective amount of an anti-angiogenic agent to treat the cancer.

46. The method of claim 45 wherein the anti-angiogenic agent is an antibody specifically binding vascular endothelial growth factor.

47. A method of treating bone destruction or bone loss associated with a tumor metastasis in a mammalian patient comprising administering to the patient an effective amount of a TGF-beta antagonist.

48. The method of claim 47 wherein the TGF-beta antagonist is an antibody specifically binding TGF-beta.

49. A method for treating a mammalian patient diagnosed with cancer comprising administering to the patient an effective amount of a combination of a TGF-beta antagonist and a chemotherapeutic or cytotoxic agent, and monitoring the response of the patient to the combination, wherein the effective amount of said combination is lower than the sum of the effective amounts of said TGF-beta antagonist and said chemotherapeutic or cytotoxic agent when administered individually, as single agents.

50. The method of claim 49 wherein the cancer is breast or colorectal cancer.

51. The method of claim 49 wherein the cancer is metastatic breast cancer.

52. The method of claim 49 wherein the chemotherapeutic agent is a taxoid.

53. The method of claim 49 wherein the patient is additionally treated with an effective dose of radiation therapy.

54. A method for treating a mammalian patient diagnosed with cancer comprising administering to the patient an effective amount of a combination of a TGF-beta antagonist and radiation therapy, wherein the effective amount of said combination is lower than the sum of the effective amounts of said TGF-beta antagonist and said radiation therapy when administered individually, as single agents.

55. The method of claim 54 wherein the cancer is breast cancer.

56. The method of claim 54 wherein the cancer is colorectal cancer.

57. The method of claim 54 further comprising administering to the patient an anti-angiogenic agent.

58. The method of claim 57 wherein the anti-angiogenic agent is an antibody specifically binding vascular endothelial growth factor.

59. The method of claim 54 wherein the TGF-beta antagonist is an antibody specifically binding TGF-beta.

60. A method for treating a mammalian patient diagnosed with cancer comprising administering to the patient an effective amount of a combination of a TGF-beta antagonist and an anti-angiogenic agent, and monitoring the response of the patient to the combination.

61. The method of claim 60 wherein the anti-angiogenic agent is an antibody specifically binding vascular endothelial growth factor.

62. The method of claim 60 wherein the TGF-beta antagonist is an antibody specifically binding TG F-beta.

63. The method of claim 60 additionally comprising administering to the patient an effective amount of a chemotherapeutic or cytotoxic agent.

64. The method of claim 60 wherein the effective amount of said combination is lower than the sum of the effective amounts of said TGF-beta antagonist and said anti-angiogenic agent when administered individually, as single agents.

65. A method for treating a mammalian patient diagnosed with cancer and predetermined not to respond, or to respond poorly, to a TGF-β antagonist, comprising administering to the patient an effective amount of a combination of a TGF-β antagonist and a chemotherapeutic or cytotoxic agent or radiation therapy, and monitoring the response of the patient to the combination.

66. The method of claim 65 wherein the cancer is breast cancer.

67. The method of claim 65 wherein the chemotherapeutic agent is a taxoid.

68. A kit comprising a container comprising an antibody specifically binding vascular endothelial growth factor, a container comprising an antibody specifically binding TGF-beta, and instructions for use of both antibodies in combination in effective amounts to treat cancer in a mammalian patient.


SEQUENCE LISTING (45 sequences)

SEQ ID NO: 1 - protein, 116 aa, Mus musculus
DIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSDTFGGGTKLEIKRTVA

SEQ ID NO: 2 - protein, 124 aa, Mus musculus
  Xaa 3, 5: Unknown amino acid
QVXLXQSGAELVRPGTSVKVSCKASGYAFTNYLIEWVKQRPGQGLEWIGVNNPGSGGSNYNEKFKGKATLTADKSSSTAYMQLSSLTSDDSAVYFCARSGGFYFDYWGQGTTQSPSPQPKRRAH

SEQ ID NO: 3 - protein, 116 aa, Artificial sequence
  Sequence is synthesized
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVA

SEQ ID NO: 4 - protein, 124 aa, Artificial sequence
  Sequence is synthesized
EVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSASTKGPS

SEQ ID NO: 5 - protein, 109 aa, Artificial sequence
  Sequence is synthesized
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKRT

SEQ ID NO: 6 - protein, 117 aa, Artificial sequence
  Sequence is synthesized
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRGXSFDYWGQGTLVTVSS

SEQ ID NO: 7 - protein, 11 aa, Artificial sequence
  Sequence is synthesized
RASQGISSYLA

SEQ ID NO: 8 - protein, 7 aa, Artificial sequence
  Sequence is synthesized
YASSLQS

SEQ ID NO: 9 - protein, 9 aa, Artificial sequence
  Sequence is synthesized
QQYNSYPYT

SEQ ID NO: 10 - protein, 10 aa, Artificial sequence
  Sequence is synthesized
GFTFSSYAMH

SEQ ID NO: 11 - DNA, 51 bp, Artificial sequence
  Sequence is synthesized
agagccagtcagagcgtgctgtatagttcgaatcagaagaactacctggcc

SEQ ID NO: 12 - DNA, 21 bp, Artificial sequence
  Sequence is synthesized
tgggctagtactcgcgagtct

SEQ ID NO: 13 - DNA, 24 bp, Artificial sequence
  Sequence is synthesized
caccagtatctgagctctgacaca

SEQ ID NO: 14 - DNA, 30 bp, Artificial sequence
  Sequence is synthesized
ggctacgcattcaccaactatctgatcgag

SEQ ID NO: 15 - DNA, 51 bp, Artificial sequence
  Sequence is synthesized
gttaacaatcctggatccggaggctccaactataacgagaagttcaagggg

SEQ ID NO: 16 - DNA, 24 bp, Artificial sequence
  Sequence is synthesized
tccggaggcttctacttcgactac

SEQ ID NO: 17 - DNA, 51 bp, Mus musculus
aagtccagtcaaagtgttttatacagttcaaatcagaagaactacttggcc

SEQ ID NO: 18 - protein, 17 aa, Artificial sequence
  Sequence is synthesized
RASQSVLYSSNQKNYLA

SEQ ID NO: 19 - protein, 7 aa, Artificial sequence
  Sequence is synthesized
WASTRES

SEQ ID NO: 20 - protein, 8 aa, Artificial sequence
  Sequence is synthesized
HQYLSSDT

SEQ ID NO: 21 - protein, 10 aa, Artificial sequence
  Sequence is synthesized
GYAFTNYLIE

SEQ ID NO: 22 - protein, 17 aa, Artificial sequence
  Sequence is synthesized
VNNPGSGGSNYNEKFKG

SEQ ID NO: 23 - protein, 8 aa, Artificial sequence
  Sequence is synthesized
SGGFYFDY

SEQ ID NO: 24 - protein, 17 aa, Mus musculus
KSSQSVLYSSNQKNYLA

SEQ ID NO: 25 - protein, 666 aa, Artificial sequence
  Sequence is synthesized
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGYAFTNYLIEWVRQAPGKGLEWIGVNNPGSGGSNYNEKFKGRFTISADNSKNTLYLQMNSLRAEDTAVYYCARSGGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 692 aa, Artificial sequence
  Sequence is synthesized
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGYAFTNYLIEWVRQAPGKGLEWVGVINPGSGGSNYNEKFKGRATISADNSKNTLYLQMNSLRAEDTAVYYCARSGGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKVRRPSRPAEAWPPWPNLFIAAYNGYK

SEQ ID NO: 27 - protein, 666 aa, Artificial sequence
  Sequence is synthesized
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGYAFTNYLIEWVRQAPGKGLEWIGVNNPGSGGSNYNEKFKGRATISADNSKNTLYLQMNSLRAEDTAVYYCARSGGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 666 aa, Artificial sequence
  Sequence is synthesized
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 29 - protein, 666 aa, Artificial sequence
  Sequence is synthesized
DIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSDTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLQQSGAELVRPGTSVKVSCKASGYAFTNYLIEWVKQRPGQGLEWIGVNNPGSGGSNYNEKFKGKATLTADKSSSTAYMQLSSLTSDDSAVYFCARSGGFYFDYWGQGTSVTVSSAKTTGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 666 aa, Artificial sequence
  Sequence is synthesized
DIQMTQSPSSLSASVGDRVTITCRASQSVLYSSNQKNYLAWYQQKPGKAPKLLIYYASSLQSGVPSRFSGSGSGTDFTLTISSLQPEDFATYYCHQYLSSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGYAFTNYLIEWVRQAPGKGLEWVGVNNPGSGGSNYNEKFKGRATISADNSKNTLYLQMNSLPAEDTAVYYCARSGGFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 31 - DNA, 1341 bp, Artificial sequence
  Sequence is synthesized
gaagttcagctggtggagtctggcggtggcctggtgcagccagggggctcactccgtttgtcctgtgcagcttctggctacgcattcaccaactatctgatcgagtgggtccgtcaggccccgggtaagggcctcgagtggatcggtgtaaacaatcctggatccggaggctccaactataacgagaagttcaagggccgtttcactataagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 32 - DNA, 1398 bp, Artificial sequence
  Sequence is synthesized
atgggatggtcatgtatcatcctttttctagtagcaactgcaactggagtacattcagaagttcagctggtggagtctggcggtggcctggtgcagccagggggctcactccgtttgtcctgtgcagcttctggctacgcattcaccaactatctgatcgagtgggtccgtcaggccccgggtaagggcctcgagtggatcggtgtaaacaatcctggatccggaggctccaactataacgagaagttcaagggccgtttcactataagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 33 - DNA, 1341 bp, Artificial sequence
  Sequence is synthesized
gaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttatcaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 1400 bp, Artificial sequence
  Sequence is synthesized
atgggatggtcatgtatcatcctttttctagtagcaactgcaactggagtacattcagaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttatcaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatg

SEQ ID NO: 35 - DNA, 1341 bp, Artificial sequence
  Sequence is synthesized
gaagttcagctggtggagtctggcggtggcctggtgcagccagggggctcactccgtttgtcctgtgcagcttctggctacgcattcaccaactatctgatcgagtgggtccgtcaggccccgggtaagggcctcgagtggatcggtgtaaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - DNA, 1398 bp, Artificial sequence
  Sequence is synthesized
atgggatggtcatgtatcatcctttttctagtagcaactgcaactggagtacattcagaagttcagctggtggagtctggcggtggcctggtgcagccagggggctcactccgtttgtcctgtgcagcttctggctacgcattcaccaactatctgatcgagtgggtccgtcaggccccgggtaagggcctcgagtggatcggtgtaaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 37 - DNA, 1341 bp, Artificial sequence
  Sequence is synthesized
gaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaa

SEQ ID NO: 38 - DNA, 1398 bp, Artificial sequence
  Sequence is synthesized
atgggatggtcatgtatcatcctttttctagtagcaactgcaactggagtacattcagaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgagggtctgcacaaccactacacgcagaagagcctctccctgtctccgggtaaa

SEQ ID NO: 39 - DNA, 657 bp, Artificial sequence
  Sequence is synthesized
gatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagtactcgcgagtctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 40 - DNA, 714 bp, Artificial sequence
  Sequence is synthesized
atgggatggtcatgtatcatcctttttctagtagcaactgcaactggagtacattcagatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagtactcgcgagtctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 41 - DNA, 657 bp, Artificial sequence
  Sequence is synthesized
gatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactatgctagcagtctccagtctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 42 - DNA, 714 bp, Artificial sequence
  Sequence is synthesized
atgggatggtcatgtatcatcctttttctagtagcaactgcaactggagtacattcagatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactatgctagcagtctccagtctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 43 - protein, 17 aa, Artificial sequence
  Sequence is synthesized
VINPGSGGSNYNEKFKG

SEQ ID NO: 44 - DNA, 5391 bp, Artificial sequence
  Sequence is synthesized
ttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaactggagtacattcagatatccagatgacccagtccccgagctccctgtccgcctctgtgggcgatagggtcaccatcacctgccgtgccagtcaggacatccgtaattatttgaactggtatcaacagaaaccaggaaaagctccgaaactactgatttactatacctcccgcctggagtctggagtcccttctcgcttctctggttctggttctgggacggattacactctgaccatcagtagtctgcaaccggaggacttcgcaacttattactgtcagcaaggtaatactctgccgtggacgttcggacagggcaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa

SEQ ID NO: 45 - DNA, 6135 bp, Artificial sequence
  Sequence is synthesized
attcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaactggagtacattcagaagttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttactacctatgccgatagcgtcaagggccgtttcactataagcgtagataaatccaaaaacacagcctacctgcaaatgaacagcctgcgtgctgaggacactgccgtctattattgtgctagaagcggatactacggcgatagcgactggtattttgacgtctggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagttggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccaactggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaatta